(19) United States
(12) Patent Application Publication
(10) Pub. No.: US 2026/0022122 A1
(43) Pub. Date: Jan. 22, 2026

(54) NOVEL COMPOUND FOR INHIBITING NAMPT, AND COMPOSITION INCLUDING SAME

(71) Applicants: Checkmate Therapeutics Inc., Seoul (KR); UIF (University Industry Foundation), Yonsei University, Seoul (KR)

(72) Inventors: Seung Wan KANG, Seoul (KR); Joo Young LEE, Seoul (KR); Hyun Seok KIM, Gyeonggi-do (KR); Hyun Hee KWAK, Seoul (KR); Ji Young LEE, Seoul (KR); Seung Won LEE, Seoul (KR)

(73) Assignees: Checkmate Therapeutics Inc., Seoul (KR); UIF (University Industry Foundation), Yonsei University, Seoul (KR)

(21) Appl. No.: 18/869,396

(22) PCT Filed: May 23, 2023
(86) PCT No.: PCT/KR2023/007009

§ 371 Date: Nov. 26, 2024

(30) Foreign Application Priority Data
May 26, 2022 (KR) 10-2022-0064687

(51) Int. Cl. C07D 487/04; A61K 31/437; A61K 31/519; A61P 35/00; C07D 471/04; C07D 519/00

(52) U.S. Cl. CPC C07D 487/04; A61K 31/437; A61K 31/519; A61P 35/00; C07D 471/04; C07D 519/00

(57) ABSTRACT

The present invention relates to a novel compound for inhibiting nicotinamide phosphoribosyl transferase (NamPT), a composition including the same, and various uses thereof.

### TECHNICAL FIELD

The present invention relates to a novel compound for inhibiting nicotinamide phosphoribosyl transferase (NamPT), a composition including the same, and various uses thereof.

### BACKGROUND ART

Nicotinamide adenine dinucleotide (NAD⁺) is a coenzyme that plays an important role in numerous physiologically essential processes [Ziegkel, M. Eur. J. Biochem., 267, 1550-1564, 2000]. NAD is essential for several signaling pathways, including mono-ADP-ribosylation, in both immune system and G-protein-coupled receptor signaling of various poly ADP-ribosylations in DNA repairing, and NAD is also essential for the deacetylase activity of sirtuin [Garten, A. et al., Trends in Endocrinology and Metabolism, 20, 130-138, 2008].

Nicotinamide phosphoribosyl transferase (NamPT) is an enzyme that catalyzes phosphorylation of nicotinamide, and a rate-limiting enzyme in one of two pathways that restore NAD.

There is increasing evidence that a NamPT inhibitor has efficacy as an anticancer agent. Cancer cells have a higher basal turnover of NAD, and require higher energy than normal cells. In addition, increased NamPT expression was reported in colorectal cancer [Van Beijnum, J. R. et al., Int. J. Cancer 101, 118-127, 2002], and NamPT is also reported to be involved in blood vessel formation [Kim, S. R. et al., Biochem. Biophys. Res. Commun. 357, 150-156, 2007]. A small molecule inhibitor of NamPT does not only causes the depletion of intercellular NAD⁺ levels, ultimately inducing tumor cell death [Hansen, C M et al., Anticancer Res. 20, 42111-4220, 2000], but also inhibits tumor growth in a xenograft model [Olese, U. H. et al., Mol Cancer Ther. 9, 1609-1617, 2010].

A NamPT inhibitor also has potential as a therapeutic agent in inflammatory and metabolic disorders [Galli, M. et al., Cancer Res. 70, 8-11, 2010]. For example, NamPT is a predominant enzyme in T and B lymphocytes. The selective inhibition of NamPT can suppress the development of autoimmune diseases by reducing NAD⁺ in lymphocytes, but not in cell types with other NAD⁺ synthesis pathways. A small molecule NamPT inhibitor (FK866) has been shown to inhibit the proliferation of activated T cells and induce apoptosis, and was effective in an animal model of arthritis (collagen-induced arthritis) [Busso, N. et al., Plos One 3, e2267, 2008]. NamPT activity increases NF-k transcriptional activity in human vascular endothelial cells and induces MMP-2 and MMP-9 activities, suggesting a role for NamPT inhibitors in the prevention of inflammation-mediated complications of obesity and type 2 diabetes [Adya, R. et al., Diabetes Care, 31, 758-760, 2008].

As one of the NamPT inhibitors, (E)-N-[4-(1-benzoylpiperidin-4-yl)butyl]-3-(pyridin-3-yl)-acrylamide (also known as APO866, FK866, WK175 or WK22.175, and referred to as ‘FK866’ [international nonproprietary name (INN)]) is also known as an anticancer agent. FK866 may be used to treat diseases associated with deregulated apoptosis, such as cancer. In the prior art, FK866 was demonstrated to interfere with the biosynthesis of nicotinamide adenyl dinucleotide (also known as NAD and referred to below) without any DNA damaging effect and induce apoptotic cell death.

In addition, FK866 induces apoptosis in HepG2 cells without imparting major effects on cell energy metabolism (Hasmann M, Schemainda I. FK866, a Highly Specific Noncompetitive Inhibitor of Nicotinamide Phosphoribosyl transferase, Represents a Novel Mechanism for Induction of Tumor Cell Apoptosis. Cancer Res 2003; 63:7436-7442. [PubMed: 14612543]). Rather than causing immediate cytotoxicity, the inhibition of NamPT and depletion of cellular NAD suggest that FK866 can be an effective agent against cancer cells that rely on nicotinamide to synthesize NAD. The crystal structure of NamPT-FK866 complex suggests that a compound binds to the nicotinamide-binding site of NamPT, inhibiting its activity. FK866 was confirmed in a murine renal cell carcinoma model and demonstrated antitumor, antimetastatic, and antiangiogenic activities (Drevs J, et al., Antiangiogenic potency of FK866/K22.175, a new inhibitor of intracellular NAD biosynthesis, in murine renal cell carcinoma. Anticancer Res 2003; 23:4853-4858. [PubMed:14981935]).

Drugs that inhibit NamPT may have numerous uses, other than inflammatory diseases or cancer, as described above. The depletion of NamPT expression can strongly affect the development of both T and B lymphocytes. In addition, NamPT is known to affect endothelial cells in relation to a high glucose level, oxidative stress, and aging, and also known to enable human endothelial cells during proliferation to withstand aging and oxidative stress of high glucose, and to use excess glucose productively to enable replicative longevity and angiogenic activity.

The present inventors have continuously conducted research on various compounds that exhibit preventive or therapeutic and improvement effects on NamPT-related diseases, and synthesized a novel compound and confirmed its effects, thereby completing the present invention.

### DISCLOSURE

### Technical Problem

The present invention is directed to providing a novel compound of Chemical Formula 1, an isomer thereof, a solvate thereof, a hydrate thereof, or a pharmaceutically acceptable salt thereof.

The present invention is also directed to providing a method of preparing a compound of Chemical Formula 1.

The present invention is also directed to providing a composition for preventing, improving or treating a NamPT-related disease, which comprises a compound of Chemical Formula 1, an isomer thereof, a solvate thereof, a hydrate thereof, or a pharmaceutically acceptable salt thereof as an active ingredient, in addition to a pharmaceutically acceptable carrier.

The present invention is also directed to providing a method of preventing, improving or treating a NamPT-related disease using a compound of Chemical Formula 1, an isomer thereof, a solvate thereof, a hydrate thereof, or a pharmaceutically acceptable salt thereof.

### Technical Solution

The inventors of the present invention discovered a novel compound that can inhibit the activity of nicotinamide phosphoribosyl transferase (hereinafter, referred to as ‘NamPT’), leading to the present invention.

Therefore, the present invention provides a compound of Chemical Formula 1, an isomer thereof, a solvate thereof, a hydrate thereof, or a pharmaceutically acceptable salt thereof.

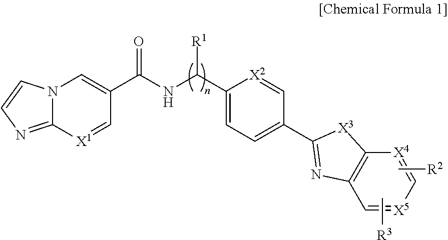

In Chemical Formula 1,
X¹ is CH or N,
X² is CH or N, and when X² is CH, it is substituted with halogen or unsubstituted,
X³ is O or S,
X⁴ and X⁵ are independently CR⁴ or N, wherein R⁴ may be the same as or different from each other,
R¹ is hydrogen or C₁₋₆ alkyl, and n is 1,
R² to R⁴ are each independently selected from the group consisting of hydrogen, halogen, amine, C₁₋₆ alkyl, C₁₋₆ haloalkyl, 5- to 10-membered aryl, 5- to 10-membered heteroaryl, —C₁₋₆ alkylene-O—R⁵, —C(O)O—R⁵, —NH—C(O)—R⁵, —NH—S(O)₂—R⁵, —S(O)₂—R⁵, —O—R⁵, and —C(O)NR⁶R⁷,
wherein R⁵ is hydrogen, C₁₋₆ alkyl, 5- to 10-membered aryl, 5- to 10-membered heteroaryl, or —C₁₋₆ alkylene-C₅₋₁₀ aryl,
R⁶ and R⁷ are each independently hydrogen, C₁₋₆ alkyl, or N, R⁶ and R⁷ are linked to each other to form 4- to 10-membered heterocycloalkyl including N.

In addition, the present invention provides a pharmaceutical composition for preventing or treating a NamPT-related disease, which comprises a compound of Chemical Formula 1, an isomer thereof, a solvate thereof, a hydrate thereof, or a pharmaceutically acceptable salt thereof as an active ingredient; and a pharmaceutically acceptable carrier.

### Advantageous Effects

With an excellent nicotinamide phosphoribosyl transferase (NamPT) inhibitory effect, a compound of Chemical Formula 1 according to the present invention can prevent, improve, or treat various NamPT-related diseases, and can be effectively used as a drug for preventing, improving or treating the diseases, especially cancer.

### MODES OF THE INVENTION

Hereinafter, the present invention will be described in detail.

Meanwhile, the embodiments of the present invention may be modified into various other forms, and the scope of the present invention is not limited to the embodiments described below. In addition, embodiments of the present invention are provided to more completely explain the present invention to one of ordinary skill in the art. Further, throughout the specification, when a certain component is “included”, it means that, unless particularly stated otherwise, another component may be further included, rather than excluding the other component.

The term “halogen” used herein may be F, Cl, Br, or I.

The term “alkyl” used herein refers to an aliphatic hydrocarbon radical. “Alkyl” may be “saturated alkyl” which does not include an alkenyl or alkynyl moiety, or a “unsaturated alkyl” which includes at least one alkenyl or alkynyl moiety.

The term “alkenyl” used herein refers to a group including at least one carbon-carbon double bond, and the term “alkynyl” refers to a group including at least one carbon-carbon triple bond.

An alkyl group may have 1 to 20 carbon atoms unless otherwise defined. In addition, an alkyl group may be medium sized alkyl having 1 to 10 carbon atoms, or lower alkyl having 1 to 6 carbon atoms. Typical alkyl groups include methyl, ethyl, propyl, isopropyl, butyl, isobutyl, 5-butyl, pentyl, hexyl, ethenyl, prophenyl, and butenyl, but the present invention is not limited thereto. For example, C₁₋₄ alkyl has 1 to 4 carbon atoms in an alkyl chain, and is selected from the group consisting of methyl, ethyl, propyl, isopropyl, n-butyl, isobutyl, sec-butyl, and t-butyl.

The term “haloalkyl” used herein may refer to —RX (X is one or more halogens (F, Cl, Br, or I)), and in other words, “haloalkyl” may be an alkyl form substituted with one or more halogens. For example, “C₁₋₈ haloalkyl” may include trifluoromethyl or difluoromethyl, but the present invention is not limited thereto.

The term “alkylene” refers to a divalent hydrocarbon group in which a radical is additionally formed from the alkyl, and for example, “alkylene” may be methylene, ethylene, propylene, butylene, or isobutylene, but the present invention is not limited thereto.

The term “alkoxy” refers to, unless otherwise defined, alkyloxy, and may be, for example, methoxy, ethoxy, or propoxy, but the present invention is not limited thereto.

The term “cycloalkyl” refers to, unless otherwise defined, a saturated or unsaturated aliphatic ring. In addition, the number of ring-forming atoms may be 3 to 12. Typical cycloalkyl groups include cyclopropyl, cyclobutyl, cyclopentyl, and cyclohexyl, but the present invention is not limited thereto.

The term “heterocycloalkyl” refers to, unless otherwise defined, the above-defined cycloalkyl which includes 1 to 3 hetero atoms selected from the group consisting of N, O and S. “Heterocycloalkyl” may be a single ring, or a multi-ring such as a spiro ring, a bridged ring, or a fused ring. Examples of heterocycloalkyl may include pyrrolidine, piperidine, tetrahydrofuran, oxetane, thiopyran, and groups similar thereto, but the present invention is not limited thereto.

The term “aryl” includes at least one ring having shared pi electron pairs, and includes, for example, a monocyclic or fused polycyclic (i.e., rings that share adjacent pairs of carbon atoms) group. That is, “aryl” used herein refers to, unless otherwise defined, a 4- to 10-membered ring including phenyl or naphthyl, and preferably, a 6- to 10-membered aromatic monocyclic or multicyclic ring.

The term “heteroaryl” refers to, unless defined otherwise, a 3- to 10-membered, preferably, 4- to 8-membered, and more preferably, 5- to 6-membered aromatic ring, which includes 1 to 3 hetero atoms selected from the group consisting of N, O, and S, and can fuse with benzo or C₃₋₈ cycloalkyl. Examples of monocyclic heteroaryl include, but are not limited to, thiazole, oxazole, thiophene, furan, pyrrole, imidazole, isoxazole, isothiazole, pyrazole, triazole, triazine, thiadiazole, tetrazole, oxadiazole, pyridine, pyridazine, pyrimidine, pyrazine, and groups similar thereto. Examples of non-cyclic heteroaryl include, but are not limited to, indole, indoline, benzothiophene, benzofuran, benzimidazile, benzoxazole, benzisoxazole, benzothiazole, benzthiadiazole, benztriazole, quinoline, isoquinoline, purine, furopyridine, and groups similar thereto.

The term “hydrate” used herein may refer to a compound of the present invention or a salt thereof, which includes a stoichiometric or non-stoichiometric amount of water bound by a non-covalent intermolecular force. A hydrate of the compound represented by Chemical Formula 1 of the present invention may include a stoichiometric or non-stoichiometric amount of water bound by a non-covalent intermolecular force. The hydrate may contain 1 equiv. or more, and preferably, 1 to 5 equiv. of water. Such a hydrate may be prepared by crystallizing the compound represented by Chemical Formula 1 of the present invention, an isomer thereof, or a pharmaceutically acceptable salt thereof from water or a solvent containing water.

The “solvate” used herein may refer to a compound of the present invention or a salt thereof, which includes a stoichiometric or non-stoichiometric amount of solvent bound by a non-covalent intermolecular force. Preferred solvents for this include those that are volatile, non-toxic, and/or suitable for administration to a human.

The term “isomer” used herein may refer to a compound of the present invention a salt thereof, which has the same chemical formula or molecular formula but structurally or sterically different. These isomers include structural isomers such as a tautomer, isomers such as R or S isomers with asymmetric carbon centers, and geometric isomers (trans and cis), and enantiomers. All these isomers and their mixtures are also included within the scope of the present invention.

The present invention provides a compound of Chemical Formula 1 below, an isomer thereof, a solvate thereof, a hydrate thereof, or a pharmaceutically acceptable salt thereof.

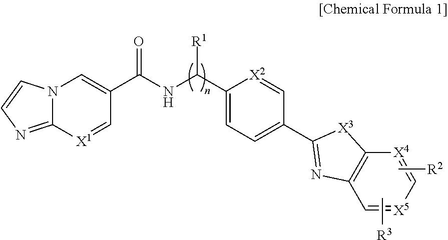

In Chemical Formula 1,
X¹ is CH or N,
X² is CH or N, and when X² is CH, it is substituted with halogen or unsubstituted,
X³ is O or S,
X⁴ and X⁵ are independently CR⁴ or N, wherein R⁴ may be the same as or different from each other,
R¹ is hydrogen or C₁₋₆ alkyl, and n is 1,
R² to R⁴ are each independently selected from the group consisting of hydrogen, halogen, amine, C₁₋₆ alkyl, C₁₋₆ haloalkyl, 5- to 10-membered aryl, 5- to 10-membered heteroaryl, —C₁₋₆ alkylene-O—R⁵, —C(O)O—R⁵, —NH—C(O)—R⁵, —NH—S(O)₂—R⁵, —S(O)₂—R⁵, —O—R⁵, and —C(O)NR⁶R⁷, wherein R⁵ is hydrogen, C₁₋₆ alkyl, 5- to 10-membered aryl, 5- to 10-membered heteroaryl, or —C₁₋₆ alkylene-C₅₋₁₀ aryl,
R⁶ and R⁷ are each independently hydrogen, C₁₋₆ alkyl, or N, R⁶ and R⁷ are linked to each other to form 4- to 10-membered heterocycloalkyl including N.

In one embodiment of the present invention, the compound of Chemical Formula 1 may be a compound represented by Chemical Formula 2 below, an isomer thereof, a solvate thereof, a hydrate thereof, or a pharmaceutically acceptable salt thereof.

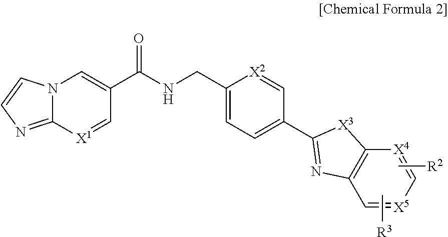

In Chemical Formula 2, X¹ to X⁵, R², and R³ are each defined as above.

In one embodiment of the present invention, the compound of Chemical Formula 1 may be a compound represented by Chemical Formula 3 below, an isomer thereof, a solvate thereof, a hydrate thereof, or a pharmaceutically acceptable salt thereof.

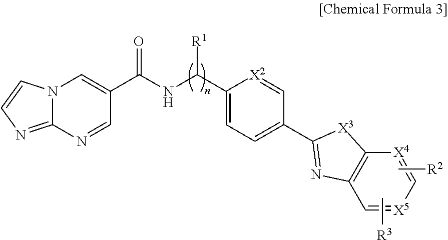

In Chemical Formula 3, X² to X⁵, n, and R¹ to R³ are each defined as above.

According to one embodiment of the present invention, in Chemical Formula 1,
X¹ is CH or N,
X² is CH or N, and when X² is CH, it is substituted with halogen or unsubstituted,
X³ is O or S,
X⁴ and X⁵ are independently CR⁴ or N, wherein R⁴ may be the same as or different from each other,
R¹ is hydrogen or C₁₋₆ alkyl, and n is 1, and
R² to R⁴ are each independently selected from the group consisting of hydrogen, halogen, amine, C₁₋₃ alkyl, C₁₋₃ haloalkyl, 5- or 6-membered aryl, 5- or 6-membered heteroaryl, —C₁₋₃ alkylene-O—R⁵, —C(O)O—R⁵, —NH—C(O)—R⁵, —NH—S(O)₂—R⁵, —S(O)₂—R⁵, —O—R⁵, and —C(O)NR⁶R⁷,
wherein R⁵ may be hydrogen, C₁₋₃ alkyl, 5- or 6-membered aryl, 5- or 6-membered heteroaryl having one or more N atoms, or —C₁₋₃ alkylene-C₅₋₆ aryl, and R⁶ and R⁷ may each be independently hydrogen, C₁₋₃ alkyl, or N, R⁶ and R⁷ may be linked to each other to form 4- to 6-membered heterocycloalkyl including N.

In one embodiment of the present invention, in Chemical Formula 1, R¹ may be hydrogen or C₁₋₆ alkyl, and specifically, hydrogen or C₁₋₃ alkyl, and is preferably hydrogen.

In one embodiment of the present invention, in Chemical Formula 1, one or more of R² to R⁴ may be hydrogen. More specifically, one, two, or three or more of R² to R⁴ may be hydrogen.

In one embodiment of the present invention, in Chemical Formula 1, in —C₁₋₃ alkylene-O—R⁵ of R² to R⁴, R⁵ may be hydrogen.

In one embodiment of the present invention, in Chemical Formula 1, in —C(O)O—R⁵ of R² to R⁴, R⁵ may be hydrogen or C₁₋₃ alkyl.

In one embodiment of the present invention, in Chemical Formula 1, in —NH—C(O)—R⁵ and —NH—S(O)₂—R⁵ of R² to R⁴, R⁵ may be C₁₋₃ alkyl.

In one embodiment of the present invention, in Chemical Formula 1, in —S(O)₂—R⁵ of R² to R⁴, R⁵ may be C₁₋₃ alkyl, or 5- or 6-membered aryl.

In one embodiment of the present invention, in Chemical Formula 1, in —O—R⁵ of R² to R⁴, R⁵ may be hydrogen, C₁₋₆ alkyl, —C₁₋₆ alkylene-C₅₋₁₀ aryl, 5- to 10-membered aryl, or 5- to 10-membered heteroaryl, more preferably, is hydrogen, C₁₋₃ alkyl, —C₁₋₃ alkylene-C₅₋₆ aryl, 5- or 6-membered aryl, or 5- or 6-membered heteroaryl including one or more N atoms, and even more preferably, is hydrogen, C₁₋₃ alkyl, phenyl, or pyridinyl.

In one embodiment of the present invention, when one of R² to R⁴ may be selected from the group consisting of amine, 5- to 10-membered aryl, 5- to 10-membered heteroaryl, —C₁₋₆ alkylene-O—R⁵, —C(O)O—R⁵, —NH—C(O)—R⁵, —NH—S(O)₂—R⁵, —S(O)₂—R⁵, and —C(O)NR⁶R⁷, the other substituents of R² to R⁴ may be hydrogen.

In one embodiment of the present invention, when X⁴ is CR⁴, wherein R⁴ is C₁₋₆ alkyl, R⁴, R² and R³ of X⁵ may be hydrogen.

In one embodiment of the present invention, in Chemical Formula 1, R⁶ and R⁷ may each be independently hydrogen, methyl, or ethyl, or N, R⁶ and R⁷ may be linked to each other to form azetidinyl or pyrrolidinyl.

In one embodiment of the present invention, in Chemical Formula 1, when X¹ is CH, R² to R⁴ may each be independently hydrogen, C₁₋₆ alkyl, or —S(O)₂—R⁵, wherein R⁵ may be C₁₋₆ alkyl.

In one embodiment of the present invention, in Chemical Formula 1, when X² is halogen-substituted C, R² to R⁴ may each be independently hydrogen or C₁₋₆ alkyl.

In one embodiment of the present invention, when X² or X⁴ is N, R² to R⁴ may each be independently hydrogen, C₁₋₆ alkyl, or halogen.

In one embodiment of the present invention, when X³ is S, R² to R⁴ may each be independently hydrogen, C₁₋₆ alkyl, C₁₋₆ alkoxy, or halogen.

In one embodiment of the present invention, when X⁵ is N, R² to R⁴ may each be independently hydrogen or halogen.

In one embodiment of the present invention, in Chemical Formula 1, X¹ may be N, X² may be CH, X³ may be O, X⁴ and X⁵ may be CR⁴, R² and R³ may each be independently selected from the group consisting of hydrogen, halogen, amine, C₁₋₃ alkyl, C₁₋₃ haloalkyl, phenyl, —C(O)OH, —S(O)₂—C₁₋₃ alkyl, and —O—R⁵, and R⁵ may be hydrogen, C₁₋₃ alkyl, phenyl, or pyridinyl,

R⁴ of X⁴ may be hydrogen, R⁴ of X⁵ may be selected from the group consisting of hydrogen, halogen, amine, C₁₋₃ alkyl, C₁₋₃ haloalkyl, —C₁₋₃ alkylene-OH, —C(O)O—C₁₋₃ alkyl, —NH—C(O)—C₁₋₃ alkyl, —NH—S(O)₂—C₁₋₃ alkyl, —S(O)₂—R⁵, —O—R⁵, and —C(O)NR⁶R⁷, and R⁵ may be hydrogen, C₁₋₃ alkyl, phenyl, pyridinyl, or —C₁₋₃ alkyl-phenyl,

R⁶ and R⁷ may each be independently hydrogen, C₁₋₆ alkyl, or N, R⁶ and R⁷ may be linked to each other to form 4- to 10-membered heterocycloalkyl including N.

The present invention also provides a method of preparing a compound of Chemical Formula 1. Hereinafter, to help understanding the present invention, the method of preparing the compound of Chemical Formula 1 is explained based on an exemplary reaction scheme. However, the compound of Chemical Formula 1 may be prepared by various methods based on the structure of Chemical Formula 1 by any one of ordinary skill in the art to which the present invention belongs, all of these methods should be construed as being included within the scope of the present invention. That is, the compound of Chemical Formula 1 may be prepared by randomly combining various synthesis methods described in the specification or disclosed in the previous art, and it is understood that this falls within the scope of the present invention, and the method of preparing the compound of Chemical Formula 1 is not limited to that described below.

In the present invention, there may be broadly two types of the methods of preparing the compound of Chemical Formula 1, and when X³ is either O or S, the compound may be prepared in different ways.

When X³ is 0, the method of preparing the compound of Chemical Formula 1 may include reacting a compound of Chemical Formula 4 with a compound of Chemical Formula 5 to prepare a compound of Chemical Formula 6; and reacting the compound of Chemical Formula 6 with a compound of Chemical Formula 7 to prepare the compound of Chemical Formula 1.

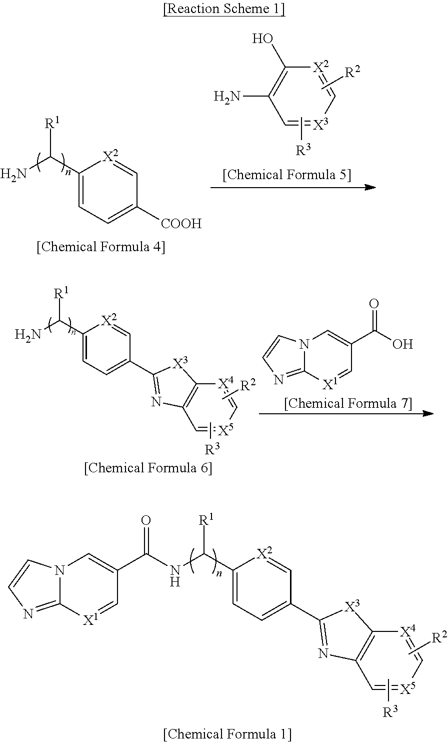

in this scheme, X¹ to X⁵, n, and R¹ to R³ may each be defined as above, and X³ is O.

The step of preparing the compound of Chemical Formula 6 by reacting the compound of Chemical Formula 4 with the compound of Chemical Formula 5 may be forming a pentagonal ring by reacting a COOH group connected to an aryl group of the compound of Chemical Formula 4 with an NH₂ group and an OH group, which are linked to an aryl group of the compound of Chemical Formula 5.

This step may include stirring the compound of Chemical Formula 4 and the compound of Chemical Formula 5 at 100 to 400° C. for 4 to 16 hours, reducing the temperature, and then adding a basic aqueous solution.

The step of preparing the compound of Chemical Formula 1 by reacting the compound of Chemical Formula 6 with the compound of Chemical Formula 7 may be forming an amide bond by reacting a NH₂ group of the compound of Chemical Formula 6 with a COOH group of the compound of Chemical Formula 7. In this step, there are no special limitations on experimental conditions (temperature, time, catalyst, etc.) for forming an amide bond, and any method of forming an amide bond with a high yield can be used without limitation.

When X³ is S, the method of preparing the compound of Chemical Formula 1 may include reacting a compound of Chemical Formula 8 with a compound of Chemical Formula 9 to prepare the compound of Chemical Formula 6; and reacting the compound of Chemical Formula 6 and the compound of Chemical Formula 7 to prepare the compound of Chemical Formula 1.

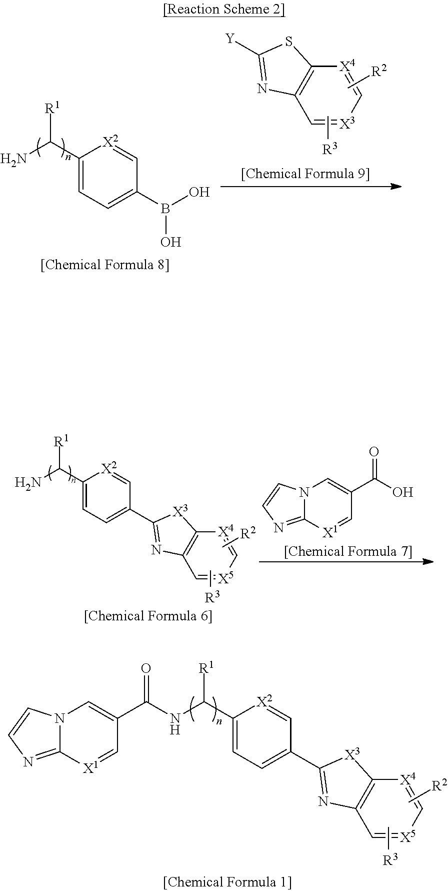

in this scheme, each of X¹ to X⁵, and R¹ to R³ is defined as above, and X³ is 5, and Y is a leaving group.

The step of preparing the compound of Chemical Formula 6 by reacting the compound of Chemical Formula 8 with the compound of Chemical Formula 9 may be a step of the Suzuki coupling reaction that connects two rings by coupling the —B(OH)₂ of the compound of Chemical Formula 8 with the leaving group Y of the compound of Chemical Formula 9. Here, the leaving group Y is not limited to the type of leaving group that can perform the Suzuki coupling reaction, and may be, for example, halogen.

When X³ is O, the step of preparing the compound of Chemical Formula 1 by reacting the compound of Chemical Formula 7 with the compound of Chemical Formula 6 may be applied in the same manner as described in the preparation method of the compound of Chemical Formula 1.

In the present invention, the compound of Chemical Formula 1 may effectively inhibit the activity of NamPT to prevent, improve, or treat a NamPT-related disease.

The present invention also provides a use of a compound of Chemical Formula 1, an isomer thereof, a solvate thereof, a hydrate thereof, or a pharmaceutically acceptable salt thereof in prevention, improvement, or treatment of a NamPT-related disease.

The present invention provides a pharmaceutical composition for preventing or treating a NamPT-related disease, which comprises the compound of Chemical Formula 1, an isomer thereof, a solvate thereof, a hydrate thereof, or a pharmaceutically acceptable salt thereof as an active ingredient; and a pharmaceutically acceptable carrier.

The pharmaceutical composition for preventing, improving or treating a NamPT-related disease may be provided by effectively inhibiting the activity of NamPT by the compound of Chemical Formula 1 of the present invention.

In the specification, “treatment” refers to stopping or delaying the progression of a disease when used on a subject showing symptoms of the disease, and “prevention” refers to stopping or delaying the onset of symptoms when used on a subject not showing symptoms but with a high risk.

In the present invention, the “pharmaceutical composition” may include a pharmaceutically acceptable carrier along with the compound of the present invention, if needed.

In the present invention, the NamPT-related disease may be one or more selected from the group consisting of cancer, viral infection, human immunodeficient virus, hepatitis virus, herpes virus, herpes simplex, inflammatory disorder, irritable bowel syndrome, inflammatory bowel disease, rheumatoid arthritis, asthma, chronic obstructive pulmonary disease, osteoarthritis, osteoporosis, dermatitis, atopic dermatitis, psoriasis, systemic lupus erythematosus, multiple sclerosis, psoriatic arthritis, ankylosing spondylitis, graft-versus-host disease, Alzheimer's disease, cerebrovascular injury, atherosclerosis, diabetes, glomerulonephritis, and metabolic syndrome, and is preferably cancer.

In the present invention, the cancer may be one or more selected from the group consisting of liver cancer, bile duct cancer, gall bladder cancer, esophageal cancer, stomach cancer, ovarian cancer, breast cancer, uterine cancer, colon cancer, rectal cancer, cervical cancer, prostate cancer, skin cancer, pancreatic cancer, leukemia, lymphoma, Hodgkin's disease, lung cancer, bronchial cancer, multiple myeloma, leukemia, lymphoma, squamous cell carcinoma, kidney cancer, urethral cancer, bladder cancer, head and neck cancer, brain cancer, and central nervous system cancer, and is preferably lung cancer or stomach cancer.

In the present invention, the pharmaceutical composition may be prepared in the form of capsule, tablet, granule, injection, ointment, powder, or beverage.

The pharmaceutical composition may be, but is not limited to, formulated in the form of an oral formulation such as a powder, granules, a capsule, a tablet or an aqueous suspension, a preparation for external use, a suppository and a sterile injectable solution according to a conventional method. The pharmaceutical composition of the present invention may include a pharmaceutically acceptable carrier. As pharmaceutically acceptable carriers, a binder, a lubricant, a disintegrant, an excipient, a solubilizer, a dispersant, a stabilizer, a suspending agent, a coloring agent and a flavor may be used for oral administration, a mixture of a buffer, a preservative, a pain relief agent, a solubilizer, an isotonic agent and a stabilizer may be used for an injectable, and a base, an excipient, a lubricant and a preservative may be used for local administration. The pharmaceutical composition of the present invention may be prepared in various forms by being mixed with the above-described pharmaceutically acceptable carrier. For example, for oral administration, the pharmaceutical composition of the present invention may be prepared in various dosage forms such as a tablet, a troche, a capsule, an elixir, a suspension, a syrup and a wafer, and for injections, the pharmaceutical composition or vaccine composition of the present invention may be prepared in a unit dose ampoule or multiple dose forms. In addition, the pharmaceutical composition of the present invention may be formulated as a solution, a suspension, a tablet, a capsule or a sustained-release preparation.

Meanwhile, examples of carriers, excipients and diluents suitable for preparation may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. The examples of carriers, excipients and diluents may also include a filler, an anti-agglomerate, a glidant, a wetting agent, a fragrance, an emulsifier, and a preservative.

The “administration” used in the present invention refers to providing the compound of the present invention to a subject by any suitable method.

The pharmaceutical composition according to the present invention is administered orally, intravenously, intramuscularly, intraarterially, intramedullary, intrathecally, intracardially, transdermally, subcutaneously, intraperitoneally, intranasally, enterally, topically, sublingually, or rectally, and preferably orally or parenterally, but the present invention is not limited thereto.

The term “parenteral” used herein means subcutaneous, intravenous, intramuscular, intraarticular, intrabursal, intrasternal, intrathecal, intralesional and intracranial injections or infusions. The pharmaceutical composition of the present invention may be administered in the form of a suppository for rectal administration.

A dose of the pharmaceutical composition of the present invention may vary according to various factors including the activity of a specific compound used, age, body weight, general health, sex, diet, administration time, administration route, excretion rate, drug formulation, and the severity of a specific disease to be prevented or treated, and a dosage of the pharmaceutical composition may be suitably selected by those of ordinary skill in the art depending on a patient's condition, body weight, the severity of a disease, a drug type, an administration route and an administration duration, and may be 0.0001 to 50 mg/kg or 0.001 to 50 mg/kg per day.

The pharmaceutical composition of the present invention may be administered once a day or several times in divided portions. The dose does not limit the scope of the present invention in any way. The pharmaceutical composition according to the present invention may be formulated as a pill, a sugar-coated tablet, a capsule, a liquid, a gel, a syrup, a slurry or a suspension.

The pharmaceutical composition of the present invention may be used alone or in combination with methods using surgery, radiotherapy, hormone therapy, chemotherapy, and biological response modifiers.

The present invention provides a method of preventing or treating a NamPT-related disease, which comprises administering a pharmaceutically effective amount of the compound of Chemical Formula 1, an isomer thereof, a solvate thereof, a hydrate thereof, or a pharmaceutically acceptable salt thereof to a subject (e.g., a human) in need of administration.

In the present invention, “subject” requiring the administration may include both mammals and non-mammals. Here, examples of the mammals include, but are not limited to, a human, and non-human primates, such as chimpanzees, and other ape and monkey species; livestock animals, such as a cow, a horse, sheep, a goat, and a pig; domesticated animals, such as a rabbit, a dog, and a cat; and laboratory animals, for example, a rodent such as a rat, a mouse, and a guinea pig. In addition, examples of the non-mammals in the present invention may include, but are not limited to, birds and fish.

In the present invention, “pharmaceutically effective amount” refers to a sufficient amount of an agent to provide a desired biological result. The results may be the reduction and/or alleviation of signs, symptoms, or causes of a disease, or any other desirable changes in the biological system. For example, an “effective amount” for therapeutic purposes is the amount of a compound disclosed in the present invention, required to provide a clinically significant decrease in a disease. The appropriate “effective” amount in any individual case may be determined by one of ordinary skill in the art using routine experimentation. Therefore, the expression “effective amount” generally refers to an amount of an active ingredient that has a therapeutic effect. In the present invention, the active ingredient is an inhibitor of the formation of NamPT.

The advantages and features of the present invention and the methods of accomplishing the same will become apparent with reference to the following examples to be described in detail and the accompanying drawings. However, the present invention is not limited to the exemplary embodiments disclosed below, and may be embodied in many different forms. These exemplary embodiments are merely provided to complete the disclosure of the present invention and fully convey the scope of the present invention to those of ordinary skill in the art, and the present invention should be defined by only the accompanying claims.

### EXAMPLES

### Preparation Example 1: Preparation of 2-amino-5-isopropyl-phenol

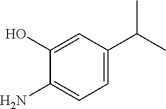

The title compounds were obtained through the processes of Steps A and B below.

### Step A: Preparation of 5-isopropyl-2-nitro-phenol

After dissolving 3-isopropylphenol (3.0 g, 22.03 mmol) in dichloromethane (40 mL), potassium nitrate (2.45 g, 24.33 mmol), sodium nitrite (75.99 mg, 1.10 mmol), and a 3M aqueous sulfuric acid solution (25.7 mL, 77.11 mmol) were added, and stirred at room temperature for 12 hours. Water (100 mL) was added to the reaction solution and extracted twice with ethyl acetate. An organic layer was dried over anhydrous sodium sulfate, filtered, concentrated under reduced pressure, and purified by column chromatography to obtain the title compound (1.2 g, 30.1%).

¹H NMR (400 MHz, CDCl₃) δ ppm 10.52-10.58 (m, 1H), 7.93 (d, 1H), 6.90 (d, 1H), 6.77 (dd, 1.81 Hz, 1H), 2.85 (m, 1H), 1.18 (d, 7H).

### Step B: Preparation of 2-amino-5-isopropyl-phenol

After dissolving the 5-isopropyl-2-nitro-phenol (1.2 g, 6.62 mmol) obtained in Step A in methanol (30 mL), palladium/carbon (100 mg) was added, and stirred in hydrogen for 12 hours. A filtrate obtained from the solid was concentrated under reduced pressure to obtain an unpurified product (960 mg, 95.9%), which was used in the next step without purification.

¹H NMR (400 MHz, CDCl₃) δ ppm 1.12 (d, 6H), 2.70 (m, 1H), 3.18-3.58 (m, 2H), 6.56-6.60 (m, 2H), 6.63-6.67 (m, 1H).

### Preparation Example 2: Preparation of 2-amino-5-chloro-4-methyl-phenol

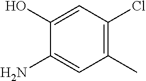

The title compounds were obtained through the processes of Steps A and B below.

### Step A: Preparation of 5-chloro-4-methyl-2-nitro-phenol

After dissolving 1-chloro-5-fluoro-2-methyl-4-nitro-benzene (7.0 g, 36.93 mmol) in dimethylformamide (35 mL), potassium acetate (9.06 g, 92.31 mmol) was added and stirred at 80° C. for 4 hours. After lowering the reaction solution to room temperature, a 1N hydrochloric acid aqueous solution (100 mL) was added and extracted twice with ethyl acetate. After washing an organic layer with water and a sodium chloride aqueous solution, a filtrate was dried over anhydrous sodium sulfate, filtered, concentrated under reduced pressure, and purified by column chromatography. After dissolving the result in methanol (15 mL), an 80% potassium hydroxide aqueous solution (25 mL) was added and stirred at 85° C. for 12 hours. After lowering the reaction solution to room temperature, a 1N hydrochloric acid aqueous solution (100 mL) was added and extracted twice with ethyl acetate. An organic layer was dried over anhydrous sodium sulfate, filtered, concentrated under reduced pressure, and purified by column chromatography to obtain the title compound (2.5 g, 36.1%).

¹H NMR (400 MHz, CDCl₃) δ ppm 2.29 (s, 3H), 7.13 (s, 1H), 7.91 (s, 1H), 10.37 (s, 1H).

### Step B: Preparation of 2-amino-5-chloro-4-methyl-phenol

The 5-chloro-4-methyl-2-nitro-phenol (1.0 g, 5.33 mmol) obtained in Step A dissolved in ethyl acetate (3 mL) was added to a suspension in which iron (1.49 g, 26.66 mmol) was added in acetic acid (5 mL) and water (8 mL), and then stirred at 80° C. for 1 hour. After lowering the reaction solution to room temperature, a 1N hydrochloric acid aqueous solution (50 mL) was added and extracted twice with ethyl acetate. An organic layer was dried over anhydrous sodium sulfate, filtered, concentrated under reduced pressure, and purified by column chromatography to obtain the title compound (420 mg, 49.9%).

¹H NMR (400 MHz, DMSO-d₆) δ ppm 2.08 (s, 3H), 4.55 (s, 2H), 6.48 (s, 1H), 6.60 (s, 1H), 9.19 (s, 1H).

### Preparation Example 3: Preparation of 2-amino-5-fluoro-4-methyl-phenol

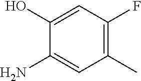

The title compounds were obtained through the processes of Steps A and B below.

### Step A: Preparation of 5-fluoro-4-methyl-2-nitro-phenol

1-Fluoro-5-methoxy-2-methyl-4-nitro-benzene (700 mg, 3.78 mmol) was dissolved in dichloromethane (25 mL) and a boron tribromide (364.29 μL, 3.78 mmol)-added dichloromethane (5 mL) solution was added, and then the resulting solution as stirred at 0° C. for 2 hours. A sodium bicarbonate aqueous solution (50 mL) was slowly added to the reaction solution and extracted twice with dichloromethane. An organic layer was washed with an aqueous sodium chloride solution, dried over anhydrous sodium sulfate, filtered, and concentrated under reduced pressure to obtain the title compound (0.64 g, 98.9%).

¹H NMR (400 MHz, CDCl₃) δ ppm 10.64 (d, 1H), 8.00 (d, 1H), 6.81 (d, 1H), 2.27 (d, 3H).

### Step B: Preparation of 2-amino-5-fluoro-4-methyl-phenol

The title compound (374 mg, 70.9%) was obtained in the same manner as in Step B of Preparation Example 2 using the 5-fluoro-4-methyl-2-nitro-phenol (0.64 g, 3.74 mmol) obtained in Step A and iron (1.04 g, 18.70 mmol).

¹H NMR (400 MHz, CDCl₃) δ ppm 6.60 (d, 1H), 6.51 (d, 1H), 2.11-2.18 (m, 3H).

### Preparation Example 4: Preparation of 2-amino-5-ethylsulfonyl-phenol

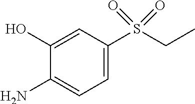

The title compounds were obtained through the processes of Steps A and B below.

### Step A: Preparation of 5-ethylsulfonyl-2-nitro-phenol

5-Fluoro-2-nitro-phenol (500 mg, 3.18 mmol) was dissolved in dimethylsulfoxide (5 mL), sodium ethanesulfinate (406.51 mg, 3.50 mmol) was added, and then the resulting solution was stirred at 100° C. for 3 hours. After lowering the reaction solution to room temperature, iced water (30 mL) was slowly added to precipitate the produced filtrate, and the resulting product was dried to obtain the title compound (520 mg, 70.7%).

¹H NMR (400 MHz, CDCl₃) δ ppm 1.35 (t, 3H), 3.19 (q, 2H), 7.53 (dd, 1H), 7.78 (d, 1H), 8.34 (d, 1H), 10.65 (s, 1H).

### Step B: Preparation of 2-amino-5-ethylsulfonyl-phenol

The title compound (360 mg, 98.5%) was obtained in the same manner as in Step B of Preparation Example 1 using the 5-ethylsulfonyl-2-nitro-phenol (420 mg, 1.82 mmol) obtained in Step A and palladium/carbon (10 mg).

¹H NMR (400 MHz, CDCl₃) δ ppm 1.17-1.20 (m, 3H), 3.02 (q, 2H), 6.67 (d, 1H), 7.20 (s, 1H), 7.30 (d, 1H).

### Preparation Example 5: Preparation of 3-amino-6-methyl-pyridin-2-ol

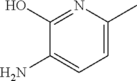

The title compound was obtained through the process of Step A below.

### Step A: Preparation of 3-amino-6-methyl-pyridin-2-ol

6-Methyl-3-nitro-pyridin-2-ol (2.0 g, 12.98 mmol) was dissolved in ethanol (10 mL) and water (20 mL), ammonium chloride (3.47 g, 64.88 mmol) and iron (2.90 g, 51.91 mmol) were added, and then the resulting solution was stirred at 80° C. for 12 hours. After lowering the reaction solution to 0° C., water (200 mL) was added and then extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (1.29 g, 80.1%).

MS [M+H]=125 (M+1)

### Preparation Example 6: Preparation of 2-amino-4-(2-pyridyloxy)phenol

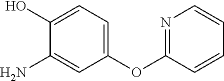 [SMILES: Nc1cc(Oc2ccccn2)ccc1O]

The title compounds were obtained through the processes of Steps A, B and C below.

### Step A: Preparation of 4-(2-pyridyloxy)phenol

Benzene-1,4-diol (6.8 g, 61.8 mmol) was dissolved in dimethylsulfoxide (50 mL), 2-fluoropyridine (3.0 g, 30.9 mmol) and NaOH (2.6 g, 64.89 mmol) were added, and the resulting solution was stirred at 80° C. for 12 hours. Water (200 mL) was added to the reaction solution and extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (6.0 g, 51.8%).

$$MS [ M + H ] = 188 ( M + 1 )$$

### Step B: Preparation of 2-nitro-4-(2-pyridyloxy)phenol

the 4-(2-pyridyloxy)phenol (5.5 g, 29.38 mmol) obtained in Step A was dissolved in acetic acid (50 mL), nitric acid (2.22 mL, purity: 68%, 33.56 mmol) was slowly added at 0° C., and then the resulting solution was stirred at room temperature for 12 hours. Water (100 mL) was added to the reaction solution, and then extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (2.7 g, 39.6%).

¹H NMR (400 MHz, DMSO-d₆) δ ppm 10.92 (s, 1H), 8.14 (dd, 1H), 7.86 (m, 1H), 7.68 (d, 1H), 7.39 (dd, 1H), 7.12-7.18 (m, 2H), 7.07 (d, 1H).

### Step C: Preparation of 2-amino-4-(2-pyridyloxy)phenol

The title compound (0.41 g, 67.5%) was obtained in the same manner as in Step B using the 2-nitro-4-(2-pyridyloxy)phenol (0.7 g, 3.01 mmol) obtained in Step B and iron (841.79 mg, 15.07 mmol).

$$MS [ M + H ] = 203 ( M + 1 )$$

### Preparation Example 7: Preparation of 6-(aminomethyl)pyridin-3-carboxylic acid

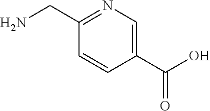 [SMILES: NCc1ccc(C(=O)O)cn1]

The title compound was obtained through the process of Step A below.

### Step A: Preparation of 6-(aminomethyl)pyridin-3-carboxylic acid

The title compound (2.26 g, unpurified) was obtained in the same manner as in Step B of Preparation Example 1 using 6-cyanopyridin-3-carboxylic acid (2.2 g, 14.85 mmol) and palladium/carbon (250 mg, 14.85 mmol).

¹H NMR (400 MHz, DMSO-d₆) δ ppm 8.90-9.06 (m, 2H), 7.49-7.58 (m, 1H), 4.10-4.30 (m, 2H).

### Preparation Example 8: Preparation of 4-(aminomethyl)-3-fluoro-benzoic acid

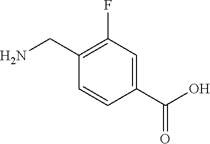 [SMILES: NCc1ccc(C(=O)O)cc1F]

The title compound was obtained through the process of Step A below.

### Step A: Preparation of 4-(aminomethyl)-3-fluoro-benzoic acid

4-Cyano-3-fluoro-benzoic acid (2.5 g, 15.14 mmol) was dissolved in methanol (30 mL), and an ammonium hydroxide aqueous solution (7.64 mL, 55.57 mmol, purity: 28%) and Raney nickel (1.95 g, 4.54 mmol, purity: 20%) were added, and then stirred under hydrogen for 16 hours. The reaction solution from which the solid was removed was concentrated under reduced pressure to obtain the title compound (1.9 g, 74.2%).

$$MS [ M + H ] = 170 ( M + 1 )$$

### Preparation Example 9: Preparation of 3-aminopyridin-4-ol

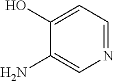

The title compound was obtained through the process of Step A below.

### Step A: Preparation of 3-aminopyridin-4-ol

The title compound (1.9 g, unpurified) was obtained in the same manner as in Step B of Preparation Example 1 using 3-nitropyridin-4-ol (2.0 g, 14.28 mmol) and palladium/carbon (0.2 g, purity: 10%).

$$MS [ M + H ] = 111 ( M + 1 )$$

### Preparation Example 10: Preparation of 2-amino-4-(benzenesulfonyl)phenol

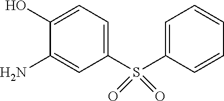 [SMILES: Nc1cc(S(=O)(=O)c2ccccc2)ccc1O]

The title compounds were obtained through the processes of Steps A and B below.

### Step A: Preparation of 4-(benzenesulfonyl)-2-nitro-phenol

2-Nitrophenol (2.0 g, 14.38 mmol) and AlCl₃ (1.18 mL, 21.57 mmol) were stirred for 15 minutes and benzenesulfonyl chloride (2.3 mL, 17.97 mmol) were added, and then the resulting solution was stirred at 140° C. for 12 hours. After lowering the reaction solution to 0° C., water (100 mL) was added and then extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (2.5 g, 62.3%).

¹H NMR (400 MHz, CDCl₃) δ ppm 10.88 (s, 1H), 8.71 (d, 1H), 8.01-8.04 (m, 1H), 8.00-8.09 (m, 1H), 7.90-7.96 (m, 2H), 7.57-7.60 (m, 1H), 7.49-7.55 (m, 2H), 7.27 (s, 1H).

### Step B: Preparation of 2-amino-4-(benzenesulfonyl)phenol

The title compound (1.65 g, 73.9%) was obtained in the same manner as in Preparation Example 5 using the 4-(benzenesulfonyl)-2-nitro-phenol (2.5 g, 8.95 mmol) obtained in Step A, ammonium chloride (2.39 g, 44.76 mmol), and iron (2.0 g, 35.81 mmol).

¹H NMR (400 MHz, DMSO-d₆) δ ppm 9.81-10.72 (m, 1H), 7.82 (d, 2H), 7.49-7.67 (m, 3H), 7.11 (d, 1H), 7.02 (dd, 1H), 6.78 (d, 1H), 5.05 (s, 2H).

### Preparation Example 11: Preparation of 2-amino-4,5-difluoro-phenol

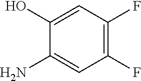

The title compound was obtained through the process of Step A below.

### Step A: Preparation of 2-amino-4,5-difluoro-phenol

The title compound (0.5 g, 60.3%) was obtained in the same manner as in Step B of Preparation Example 2 using 4,5-difluoro-2-nitro-phenol (1.0 g, 5.71 mmol) and iron (1.59 g, 28.56 mmol).

¹H NMR (400 MHz, CDCl₃) δ ppm 6.56-6.67 (m, 2H), 3.09-4.13 (m, 2H).

### Preparation Example 12: Preparation of 2-amino-3,5-difluoro-phenol

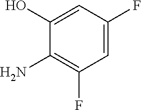

The title compound was obtained through the process of Step A below.

### Step A: Preparation of 2-amino-3,5-difluoro-phenol

The title compound (3.6 g, unpurified) was obtained in the same manner as in Preparation Example 5 using 3,5-difluoro-2-nitro-phenol (2.0 g, 11.42 mmol), iron (2.55 g, 45.68 mmol), and ammonium chloride (3.06 g, 57.11 mmol).

¹H NMR (400 MHz, DMSO-d₆) δ ppm 4.15-4.54 (m, 1H), 6.44 (d, 1H), 6.52-6.63 (m, 1H), 9.35-10.85 (m, 1H).

### Preparation Example 13: Preparation of 2-amino-5-chloro-4-(trifluoromethyl)phenol

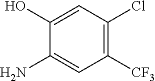 [SMILES: Nc1cc(C(F)(F)F)c(Cl)cc1O]

The title compounds were obtained through the processes of Steps A and B below.

### Step A: Preparation of 5-chloro-2-nitro-4-(trifluoromethyl)phenol

1,5-Dichloro-2-nitro-4-(trifluoromethyl)benzene (5.0 g, 19.23 mmol) was dissolved in dimethylformamide (30 mL), and potassium acetate (3.77 g, 38.46 mmol) was added, and then the resulting solution was stirred at 80° C. for 3 hours. After lowering the reaction solution to room temperature, 1N hydrochloric acid aqueous solution (300 mL) was added, and then extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (3.85 g, 82.9%).

¹H NMR (400 MHz, CDCl₃) δ ppm 10.82 (s, 1H), 8.49 (s, 1H), 7.37 (s, 1H).

### Step B: Preparation of 2-amino-5-chloro-4-(trifluoromethyl)phenol

The title compound (2.5 g, 74.1%) was obtained in the same manner as in Step B of Preparation Example 2 using the 5-chloro-2-nitro-4-(trifluoromethyl)phenol (3.85 g, 15.94 mmol) obtained in Step A and iron (4.45 g, 79.69 mmol).

¹H NMR (400 MHz, CDCl₃) δ ppm 7.04 (s, 1H), 6.84 (s, 1H), 4.16 (m, 2H).

### Preparation Example 14: Preparation of 2-amino-4-chloro-5-(trifluoromethyl)phenol

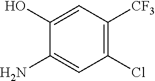 [SMILES: Nc1cc(Cl)c(C(F)(F)F)cc1O]

The title compounds were obtained through the processes of Steps A and B below.

### Step A: Preparation of 4-chloro-2-nitro-5-(trifluoromethyl)phenol

4-Chloro-3-(trifluoromethyl)phenol (2.0 g, 10.18 mmol) was dissolved in acetic acid (10 mL), sulfuric acid (1.5 mL) was slowly added at 0° C., and then the resulting solution was stirred for 30 minutes. In addition, nitric acid (740 uL, 11.19 mmol, purity: 68%) was added and stirred at room temperature for 12 hours. Cold water (60 mL) was added to the reaction solution and extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (765 mg, 31.1%).

¹H NMR (400 MHz, CDCl₃) δ ppm 10.43 (s, 1H), 8.27 (s, 1H), 7.58 (s, 1H).

### Step B: Preparation of 2-amino-4-chloro-5-(trifluoromethyl)phenol

The title compound (650 mg, unpurified) was obtained in the same manner as in Step B of Preparation Example 2 using the 4-chloro-2-nitro-5-(trifluoromethyl)phenol (0.74 g, 3.06 mmol) obtained in Step A and iron (855.41 mg, 15.32 mmol).

¹H NMR (400 MHz, CDCl₃) δ ppm 7.00 (s, 1H), 6.78 (s, 1H), 3.99-4.28 (m, 2H).

### Preparation Example 15: Preparation of 2-amino-4-methoxy-5-methyl-phenol

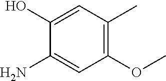

The title compound was obtained through the processes of Steps A, B, and C.

### Step A: Preparation of 1,4-dimethoxy-2-methyl-5-nitro-benzene

The title compound (6.44 g, 99.4%) was obtained in the same manner as in Step B of Preparation Example 5 using 1,4-dimethoxy-2-methyl-benzene (5.0 g, 32.82 mmol) and nitric acid (2.8 mL, 40.44 mmol, purity: 65%).

¹H NMR (400 MHz, CDCl₃) δ ppm 7.41 (s, 1H), 6.91 (s, 1H), 3.93 (s, 3H), 3.85 (s, 3H), 2.29 (s, 3H).

### Step B: Preparation of 4-methoxy-5-methyl-2-nitro-phenol

The 1,4-dimethoxy-2-methyl-5-nitro-benzene (3.0 g, 15.21 mmol) obtained in Step A was dissolved in dichloromethane (60 mL), boron trichloride (1M, 15.97 mL, 15.97 mmol) was added at −20° C., and then the resulting solution was stirred at room temperature for 12 hours. A sodium carbonate aqueous solution (200 mL) was added to the reaction solution and extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (2.3 g, 82.5%).

¹H NMR (400 MHz, CDCl₃) δ ppm 10.47 (s, 1H), 7.40 (s, 1H), 6.95 (s, 1H), 3.85 (s, 3H), 2.27 (s, 3H).

### Step C: Preparation of 2-amino-4-methoxy-5-methyl-phenol

The title compound (1.45 g, 75.4%) was obtained in the same manner as in Step B of Preparation Example 1 using the 4-methoxy-5-methyl-2-nitro-phenol (2.3 g, 12.56 mmol) obtained in Step B and palladium/carbon (0.4 g, 375.87 mol, purity: 10%).

¹H NMR (400 MHz, CDCl₃) δ ppm 6.58 (d, 1H), 6.35 (d, 1H), 3.75 (d, 3H), 2.07-2.14 (m, 3H).

### Preparation Example 16: Preparation of 2-amino-4-ethyl-phenol

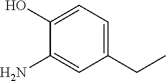

The title compounds were obtained through the processes of Steps A and B below.

### Step A: Preparation of 4-ethyl-2-nitro-phenol

The title compound (0.94 g, 34.3%) was obtained in the same manner as in Step A of Preparation Example 14 using 4-ethylphenol (2.0 g, 16.37 mmol), sulfuric acid (1.5 mL), and nitric acid (1.49 mL, 22.45 mmol, purity: 68%).

¹H NMR (400 MHz, CDCl₃) δ ppm 10.47 (s, 1H), 7.91-7.93 (m, 1H), 7.44 (dd, 1H), 7.07-7.11 (m, 1H), 2.65 (m, 2H), 1.23-1.28 (m, 3H).

### Step B: Preparation of 2-amino-4-ethyl-phenol

The title compound (0.584 g, 84.7%) was obtained in the same manner as in Step B of Preparation Example 2 using the 4-ethyl-2-nitro-phenol (0.84 g, 5.03 mmol) obtained in Step A and iron (1.4 g, 23.15 mmol).

¹H NMR (400 MHz, CDCl₃) δ ppm 6.66 (d, 1H), 6.62 (s, 1H), 6.52 (d, 1H), 3.67-4.30 (m, 2H), 2.52 (m, 2H), 1.17-1.22 (m, 3H).

### Preparation Example 17: Preparation of 2-amino-5-ethyl-phenol

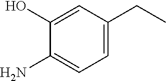

### Step A: Preparation of 5-ethyl-2-nitro-phenol

The title compound (2.9 g, 30.3%) was obtained in the same manner as in Step A of Preparation Example 6 using 3-ethylphenol (7.0 g, 57.3 mmol) and nitric acid (4.15 mL, 62.7 mmol, purity: 68%).

¹H NMR (400 MHz, CDCl₃) δ ppm 10.64 (s, 1H), 8.01 (d, 1H), 6.97 (s, 1H), 6.82 (dd, 1H), 2.69 (m, 2H), 1.27 (t, 3H).

### Step B: Preparation of 2-amino-5-ethyl-phenol

The title compound (1.46 g, 61.4%) was obtained in the same manner as in Step B of Preparation Example 2 using the 5-ethyl-2-nitro-phenol (2.9 g, 17.35 mmol) obtained in Step A and iron (4.84 g, 86.74 mmol).

¹H NMR (400 MHz, CDCl₃) δ ppm 6.71-6.74 (m, 1H), 6.61-6.64 (m, 2H), 3.47-4.16 (m, 2H), 2.52 (m, 2H), 1.18 (t, 3H).

### Preparation Example 18: Preparation of 2-bromo-5-methyl-1,3-benzothiazole

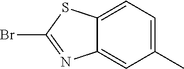

The title compound was obtained through the process of Step A below.

### Step A: Preparation of 2-bromo-5-methyl-1,3-benzothiazole

5-Methyl-1,3-benzothiazole (800 mg, 5.36 mmol) was dissolved in tetrahydrofuran (8 mL) under nitrogen, n-butyl lithium (2.5M, 2.68 mL, 6.70 mmol) was added at −70° C., and then stirred at −70° C. for 1 hour. In addition, N-bromosuccinimide (1.19 g, 6.70 mmol) dissolved in tetrahydrofuran (4 mL) was added, and then stirred at −70° C. for 1 hour. Water was added to the reaction solution and then extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (0.36 g, 29.4%).

MS [M+H]=229 (M+1)

### Preparation Example 19: Preparation of 2-bromo-5-methoxy-1,3-benzothiazole

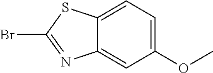

The title compound was obtained through the process of Step A below.

### Step A: Preparation of 2-bromo-5-methoxy-1,3-benzothiazole

Bromine (376.29 μL, 7.30 mmol) was dissolved in chloroform (4 mL), 5-methoxy-1,3-benzothiazole-2-thiol (400 mg, 2.03 mmol) dissolved in chloroform was slowly added at 0° C. for 1 hour, and then the resulting solution was stirred at room temperature for 30 minutes. After lowering the reaction solution to 0° C., water (100 mL) was added thereto, and then extraction was performed twice with dichloromethane. After washing an organic layer with a sodium chloride aqueous solution, a filtrate dried and filtered with anhydrous sodium sulfate was concentrated under reduced pressure and purified by column chromatography to obtain the title compound (0.258 g, 52.2%).

¹H NMR (400 MHz, DMSO-d₆) δ ppm 7.95-8.00 (m, 1H), 7.55 (d, 1H), 7.14 (dd, J=8.80, 2.40 Hz, 1H), 3.84 (s, 3H).

### Preparation Example 20: Preparation of 2-amino-4-phenoxy-phenol

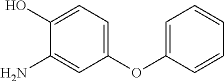 [SMILES: Nc1cc(Oc2ccccc2)ccc1O]

The title compounds were obtained through the processes of Steps A and B below.

### Step A: Preparation of 2-nitro-4-phenoxy-phenol

The title compound (2.2 g, 35.4%) was obtained in the same manner as in Step B of Preparation Example 5 using 4-phenoxyphenol (5.0 g, 26.85 mmol) and nitric acid (1.84 mL, 27.84 mmol, purity: 68%).

¹H NMR (400 MHz, CDCl₃) δ ppm 10.39 (s, 1H), 7.70 (d, J=2.80 Hz, 1H), 7.33-7.41 (m, 3H), 7.14-7.19 (m, 2H), 7.00 (d, J=8.00 Hz, 2H).

### Step B: Preparation of 2-amino-4-phenoxy-phenol

The title compound (1.7 g, 98.4%) was obtained in the same manner as in Step B of Preparation Example 2 using the 2-nitro-4-phenoxy-phenol (2.1 g, 9.08 mmol) obtained in Step A and iron (2.54 g, 45.41 mmol).

¹H NMR (400 MHz, DMSO-d₆) δ ppm 8.06 (s, 1H), 6.39-6.48 (m, 2H), 6.12-6.19 (m, 1H), 6.03 (dd, 2H), 5.76 (d, 1H), 5.44 (d, 1H), 5.22 (dd, 1H), 3.84 (s, 2H).

### Preparation Example 21: Preparation of 2-amino-4-propoxy-phenol

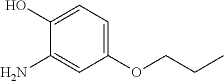

The title compounds were obtained through the processes of Steps A and B below.

### Step A: Preparation of 2-nitro-4-propoxy-phenol

2-Nitro-1,4-dipropoxy-benzene (2.2 g, 9.19 mmol) was dissolved in chloroform (45 mL), aluminum chloride (1.84 g, 13.79 mmol) was added at 0° C., and then the resulting solution was stirred at room temperature for 2 hours. After lowering the reaction solution to 0° C., 2N hydrochloric aqueous solution (5 mL) was added and then extraction was performed twice with dichloromethane. After washing an organic layer with a sodium chloride aqueous solution, a filtrate dried and filtered with anhydrous sodium sulfate was concentrated under reduced pressure and purified by column chromatography to obtain the title compound (1.6 g, 88.3%).

¹H NMR (400 MHz, DMSO-d₆) δ ppm 10.25-10.56 (m, 1H), 7.38 (d, 1H), 7.18-7.22 (m, 1H), 7.04-7.08 (m, 1H), 3.89-3.93 (m, 2H), 1.66-1.75 (m, 2H), 0.94-0.99 (m, 3H).

### Step B: Preparation of 2-amino-4-propoxy-phenol

The title compound (290 mg, 28.5%) was obtained in the same manner as in Preparation Example 5 using the 2-nitro-4-propoxy-phenol (1.2 g, 6.09 mmol) obtained in Step A, iron (1.7 g, 30.43 mmol), and ammonium chloride (2.6 g, 48.68 mmol).

$$MS [ M + H ] = 168 ( M + 1 )$$

### Preparation Example 22: Preparation of 2-amino-4-benzyloxyl-phenol

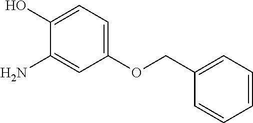 [SMILES: Nc1cc(OCc2ccccc2)ccc1O]

### Step A: Preparation of 4-benzyloxy-2-nitro-phenol

1,4-Dibenzyloxy-2-nitro-benzene (5.7 g, 17.0 mmol) was dissolved in dichloromethane (50 mL), trifluoroacetic acid (5.7 mL, 76.98 mmol) was added, and the resulting solution was stirred at room temperature for 48 hours. The reaction solution was concentrated under reduced pressure to obtain a residue and then purified by column chromatography to obtain the title compound (4.5 g, 92.6%).

¹H NMR (400 MHz, CDCl₃) δ ppm 10.35 (s, 1H), 7.62 (d, 1H), 7.34-7.45 (m, 5H), 7.30 (dd, 1H), 7.11 (d, 1H), 5.08 (s, 2H).

### Step B: Preparation of 2-amino-4-benzyloxy-phenol

The 4-benzyloxy-2-nitro-phenol (2.0 g, 8.16 mmol) obtained in Step A was dissolved in ethanol (50 mL) and water (50 mL), sodium hyposulfite (5.68 g, 32.62 mmol) was added, and then the resulting solution was stirred under nitrogen at 75° C. for 1 hour. Water (200 mL) was added to the reaction solution, and extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (930 mg, 52.9%).

¹H NMR (400 MHz, CDCl₃) δ ppm 7.29-7.48 (m, 5H), 6.65 (d, 1H), 6.43 (s, 1H), 6.29 (d, 1H), 4.98 (s, 2H).

### Preparation Example 23: Preparation of 2-amino-5-phenoxy-phenol

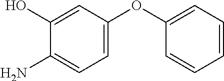 [SMILES: Nc1ccc(Oc2ccccc2)cc1O]

The title compounds were obtained through the processes of Steps A and B below.

### Step A: Preparation of 2-nitro-5-phenoxy-phenol

5-Fluoro-2-nitro-phenol (10 g, 63.65 mmol) was dissolved in N-methyl-2-pyrrolidone (100 mL), phenol (7.19 g, 76.38 mmol) and calcium carbonate (11.44 g, 82.75 mmol) were added, and then the resulting solution was stirred at 160° C. for 5 hours. After lowering the reaction solution to room temperature and adjusting 12N hydrochloric acid aqueous solution to approximately pH 3 to 4, water (400 mL) was added and then the resulting solution was extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (13.81 g, 93.8%).

¹H NMR (400 MHz, CDCl₃) δ ppm 10.90 (s, 1H), 8.07-8.10 (m, 1H), 7.43-7.49 (m, 2H), 7.30 (dt, 1H), 7.09-7.14 (m, 2H), 6.58-6.62 (m, 1H), 6.51-6.54 (m, 1H).

### Step B: Preparation of 2-amino-5-phenoxy-phenol

The title compound (2.6 g, 59.8%) was obtained in the same manner as in Preparation Example 5 using the 2-nitro-5-phenoxy-phenol (5 g, 21.63 mmol) obtained in Step A, iron (6.04 g, 108.13 mmol), and ammonium chloride (9.25 g, 173.01 mmol).

¹H NMR (400 MHz, DMSO-d₆) δ ppm 8.13 (s, 1H), 7.30 (t, 2H), 7.00 (t, 1H), 6.84-6.89 (m, 2H), 6.58 (d, 1H), 6.38 (d, 1H), 6.27-6.31 (m, 1H).

### Preparation Example 24: Preparation of 2-amino-5-(3-pyridyloxy)phenol

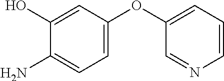 [SMILES: Nc1ccc(Oc2cccnc2)cc1O]

The title compound was obtained through the processes of Steps A, B, and C.

### Step A: Preparation of 3-(3-methoxy-4-nitro-phenoxy)pyridine

The title compound (4.9 g, 68.1%) was obtained in the same manner as in Step A of Preparation Example 23 using 4-fluoro-2-methoxy-1-nitro-benzene (5.0 g, 29.22 mmol), pyridin-2-ol (3.61 g, 37.98 mmol), and potassium carbonate (6.06 g, 43.83 mmol).

¹H NMR (400 MHz, CDCl₃) δ ppm 8.51-8.53 (m, 1H), 8.49 (d, 1H), 7.96 (d, 1H), 7.37-7.46 (m, 2H), 6.71 (d, 1H), 6.52 (dd, 1H), 3.93 (s, 3H).

### Step B: Preparation of 2-methoxy-4-(3-pyridyloxy)aniline

The title compound (3.8 g, 92.1%) was obtained in the same manner as in Preparation Example 5 using the 3-(3-methoxy-4-nitro-phenoxy)pyridine (4.7 g, 19.09 mmol) obtained in Step A, iron (4.26 g, 76.35 mmol), and ammonium chloride (5.11 g, 95.44 mmol).

$$MS [ M + H ] = 217 ( M + 1 )$$

### Step C: Preparation of 2-amino-5-(3-pyridyloxy)phenol

The title compound (740 mg, 79.1%) was obtained in the same manner as in Step A of Preparation Example 3 using the 3-(3-methoxy-4-nitro-phenoxy)pyridine (1.0 g, 4.62 mmol) obtained in Step B, and boron tribromide (534.72 μL, 5.55 mmol).

¹H NMR (400 MHz, CDCl₃) δ ppm 8.24-8.26 (m, 1H), 8.16 (s, 1H), 7.31-7.36 (m, 1H), 7.28 (d, 1H), 6.74 (d, 1H), 6.48 (d, 1H), 6.38 (d, 1H).

### Preparation Example 25: Preparation of 3-amino-6-chloro-pyridin-2-ol

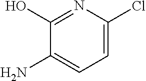

The title compound was obtained through the process of Step A below.

### Step A: Preparation of 3-amino-6-chloro-pyridin-2-ol

The title compound (340 mg, 20.5%) was obtained in the same manner as in Preparation Example 5 using 6-chloro-3-nitro-pyridin-2-ol (2.0 g, 11.46 mmol), iron (2.56 g, 45.68 mmol), and ammonium chloride (3.06 g, 57.11 mmol).

$$MS [ M + H ] = 145 ( M + 1 )$$

### Preparation Example 26: Preparation of 5-amino-2-chloro-pyridin-4-ol

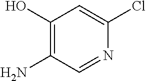

The title compounds were obtained through the processes of Steps A and B below.

### Step A: Preparation of 2-chloro-5-nitro-pyridin-4-ol

2-Chloro-4-methoxy-5-nitro-pyridine (1.0 g, 5.3 mmol) was dissolved in a hydrochloric acid solution (8 mL, purity: 36%) and then stirred at 100° C. for 12 hours. The reaction solution was concentrated under reduced pressure and dried to obtain an unpurified product (1.0 g), and used in a subsequent reaction without purification.

$$MS [ M + H ] = 175 ( M + 1 )$$

### Step B: Preparation of 5-amino-2-chloro-pyridin-4-ol

The 2-chloro-5-nitro-pyridin-4-ol (1.0 g, 5.73 mmol) obtained in Step A was dissolved in ethyl acetate (15 mL), stannous chloride dihydrate (9.05 g, 40.11 mmol) was added, and then the resulting solution was stirred under nitrogen at 80° C. for 3 hours. Water (100 mL) was stirred to the reaction solution and neutralized with a sodium bicarbonate solution, and then the resulting solution as extracted twice with ethyl acetate. After washing an organic layer with a sodium chloride aqueous solution, a filtrate dried and filtered with anhydrous sodium sulfate was concentrated under reduced pressure and purified by column chromatography to obtain the title compound (0.8 g, 96.6%).

¹H NMR (400 MHz, DMSO-d₆) δ ppm 7.88 (s, 1H), 7.19 (s, 1H), 3.92-4.20 (m, 2H).

Compounds of Examples of the present invention were prepared as below using the compounds prepared according to Preparation Examples.

### Example 1: Preparation of N-[[4-(6-methyl-1,3-benzoxazol-2-yl)phenyl]methyl]imidazo[1,2-a]pyrimidin-6-carboxamide

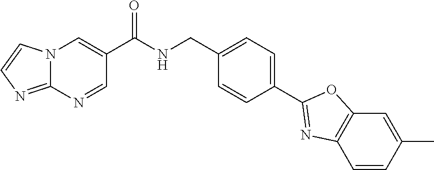 [SMILES: Cc1ccc2nc(-c3ccc(CNC(=O)c4cnc5nccn5c4)cc3)oc2c1]

### Step A: Preparation of [4-(6-methyl-1,3-benzoxazol-2-yl)phenyl]methanamine

4-(Aminomethyl)benzoic acid (5.0 g, 33.08 mmol) and 2-amino-5-methyl-phenol (4.07 g, 33.08 mmol) were dissolved in polyphosphoric acid (20 mL), and then stirred at 200° C. for 8 hours. After lowering the reaction solution to 60° C., an 1N sodium hydroxide aqueous solution (100 mL) was added and then the resulting solution was extracted twice with ethyl acetate. After washing an organic layer with a sodium chloride aqueous solution, a filtrate dried and filtered with anhydrous sodium sulfate was concentrated under reduced pressure and purified through column chromatography to obtain the title compound (5.4 g, 81.1%).

¹H NMR (400 MHz, CDCl₃) δ ppm 8.21 (d, 2H), 7.64 (d, 1H), 7.48 (d, 2H), 7.39 (s, 1H), 7.17 (dd, 1H), 3.97 (s, 2H), 2.51 (s, 3H).

### Step B: Preparation of N-[[4-(6-methyl-1,3-benzoxazol-2-yl)phenyl]methyl]imidazo[1,2-a]pyrimidin-6-carboxamide

the [4-(6-methyl-1,3-benzoxazol-2-yl)phenyl]methanamine (146.07 mg, 612.99 μmol) obtained in Step A, imidazo[1,2-a]pyrimidin-6-carboxylic acid (100 mg, 612.99 μmol), and 1-ethyl-3-(3-dimethylaminopropyl)carbodiimide (141.01 mg, 735.59 μmol) were dissolved in pyridine (3 mL) and stirred at 50° C. for 12 hours. After a residue was obtained by concentrating the reaction solution under reduced pressure, it was purified by prep-HPLC to obtain the title compound (16.55 mg, 7.0%).

¹H NMR (400 MHz, DMSO-d₆) δ ppm 9.02 (t, 1H), 8.54 (d, 1H), 8.14 (d, 2H), 8.03 (dd, 1H), 7.66 (d, 1H), 7.59 (s, 1H), 7.53 (d, 2H), 7.22 (dd, 1H), 7.16 (s, 1H), 6.65 (d, 1H), 4.55 (d, 2H), 2.46 (s, 3H).

### Example 2: Preparation of N-[[4-(1,3-benzoxazol-2-yl)phenyl]methyl]imidazo[1,2-a]pyrimidin-6-carboxamide

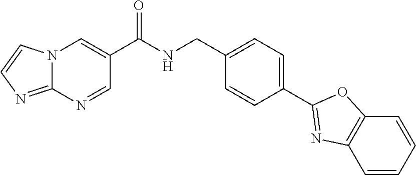 [SMILES: O=C(NCc1ccc(-c2nc3ccccc3o2)cc1)c1cnc2nccn2c1]

The title compounds were obtained through the processes of Steps A and B below.

### Step A: Preparation of [4-(1,3-benzoxazol-2-yl)phenyl]methanamine

The title compound (1.0 g, 67.4%) was obtained in the same manner as in Step A of Example 1 using 4-(aminomethyl)benzoic acid (1.0 g, 6.62 mmol) and 2-amino-phenol (722.4 mg, 6.62 mmol).

¹H NMR (400 MHz, CDCl₃) δ ppm 8.14-8.18 (m, 2H), 7.67-7.72 (m, 1H), 7.49-7.53 (m, 1H), 7.38-7.44 (m, 2H), 7.25-7.31 (m, 2H), 3.88-3.95 (m, 2H).

### Step B: Preparation of N-[[4-(1,3-benzoxazol-2-yl)phenyl]methyl]imidazo[1,2-a]pyrimidin-6-carboxamide

imidazo[1,2-a]pyrimidin-6-carboxylic acid (0.15 g, 919.49 μmol) was dissolved in dichloromethane (5 mL), oxalyl chloride (643.90 μL, 7.36 mmol) and dimethyl formamide (3.54 μL, 45.97 μmol) were slowly added at 0° C., and the resulting solution was stirred at 20° C. for 2 hours. The reaction solution was concentrated under reduced pressure and then dissolved in dichloromethane (5 mL), the [4-(1,3-benzoxazol-2-yl)phenyl]methanamine (103.1 mg, 459.75 μmol) obtained in Step A and triethylamine (1.28 mL, 9.19 mmol) were added, and then the resulting solution was stirred at room temperature for 12 hours. The reaction solution was concentrated under reduced pressure to obtain a residue, which was then purified by prep-HPLC to obtain the title compound (23.3 mg, 13.7%).

¹H NMR (400 MHz, DMSO-d₆) δ ppm 9.54 (d, 1H), 9.42 (t, 1H), 8.99 (d, 1H), 8.20 (d, 2H), 8.05 (d, 1H), 7.77-7.85 (m, 3H), 7.62 (d, 2H), 7.37-7.48 (m, 2H), 4.65 (d, 2H).

### Example 3: Preparation of N-[[4-(1,3-benzoxazol-2-yl)phenyl]methyl]imidazo[1,2-a]pyrimidin-6-carboxamide

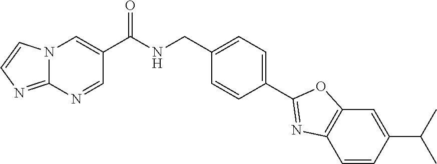 [SMILES: CC(C)c1ccc2nc(-c3ccc(CNC(=O)c4cnc5nccn5c4)cc3)oc2c1]

The title compounds were obtained through the processes of Steps A and B below.

### Step A: Preparation of [4-(6-isopropyl-1,3-benzoxazol-2-yl)phenyl]methanamine

The title compound (1.0 g, 59.1%) was obtained in the same manner as in Step A of Example 1 using 4-(aminomethyl)benzoic acid (959.7 mg, 6.35 mmol) and the 2-amino-5-isopropyl-phenol (960 mg, 6.35 mmol) obtained in Preparation Example 1.

¹H NMR (400 MHz, CDCl₃) δ ppm 1.25 (d, 6H), 3.00 (m, 1H), 3.90 (s, 2H), 7.16 (m, 1H), 7.36-7.43 (m, 3H), 7.59 (d, 1H), 8.13 (d, 2H).

### Step B: Preparation of N-[[4-(1,3-benzoxazol-2-yl)phenyl]methyl]imidazo[1,2-a]pyrimidin-6-carboxamide

After dissolving imidazo[1,2-a]pyrimidin-6-carboxylic acid (150 mg, 919.49 μmol) in acetonitrile (5 mL), [chloro(dimethylamino)methylene]-dimethyl-ammonium hexafluorophosphate (309.6 mg, 1.10 mmol), the [4-(6-isopropyl-1,3-benzoxazol-2-yl)phenyl]methanamine (244.9 mg, 919.49 μmol) obtained in Step A, and 1-methylimidazole (264.2 mg, 3.22 mmol) were sequentially added and then the resulting solution was stirred at room temperature for 12 hours. The reaction solution was concentrated under reduced pressure and purified by prep-HPLC to obtain the title compound (62.8 mg, 16.3%).

¹H NMR (400 MHz, DMSO-d₆) δ ppm 1.27 (d, 6H), 3.02-3.11 (m, 1H), 4.64 (d, 2H), 7.31 (m, 1H), 7.60 (d, 2H), 7.64-7.72 (m, 2H), 7.83 (d, 1H), 8.05 (d, 1H), 8.16 (d, 2H), 9.00 (d, 1H), 9.41 (t, 1H), 9.54 (d, 1H).

### Example 4: Preparation of N-[[4-(6-chloro-1,3-benzoxazol-2-yl)phenyl]methyl]imidazo[1,2-a]pyrimidin-6-carboxamide

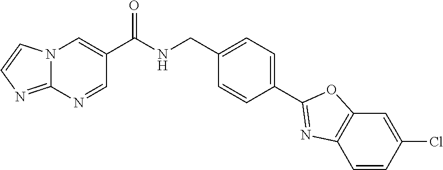 [SMILES: O=C(NCc1ccc(-c2nc3ccc(Cl)cc3o2)cc1)c1cnc2nccn2c1]

The title compounds were obtained through the processes of Steps A and B below.

### Step A: Preparation of [4-(6-chloro-1,3-benzoxazol-2-yl)phenyl]methanamine

The title compound (1.11 g, 64.9%) was obtained in the same manner as in Step A of Example 1 using 4-(aminomethyl)benzoic acid (1.0 g, 6.62 mmol) and 2-amino-5-chloro-phenol (949.78 mg, 6.62 mmol).

¹H NMR (400 MHz, CDCl₃) δ ppm 3.90 (s, 2H), 4.77-5.28 (m, 1H), 7.26 (m, 1H), 7.41 (br d, 2H), 7.51 (d, 1H), 7.59 (d, 1H), 8.12 (br d, 2H).

### Step B: Preparation of N-[[4-(6-chloro-1,3-benzoxazol-2-yl)phenyl]methyl]imidazo[1,2-a]pyrimidin-6-carboxamide

After dissolving benzotriazol-1-yloxy tris(dimethylamino)phosphonium hexafluorophosphate (542.23 mg, 1.23 mmol), imidazo[1,2-a]pyrimidin-6-carboxylic acid (100 mg, 612.99 μmol), and triethylamine (186.08 mg, 1.84 mmol) in anhydrous dimethyl formamide (3 mL), the [4-(6-chloro-1,3-benzoxazol-2-yl)phenyl]methanamine (158.58 mg, 612.99 μmol) obtained in Step A was added, and the resulting solution was stirred at 40° C. for 2 hours. The reaction solution was concentrated under reduced pressure and purified by prep-HPLC to obtain the title compound (5.6 mg, 2.2%).

¹H NMR (400 MHz, DMSO-d₆) δ ppm 4.65 (d, 2H), 7.47 (m, 1H), 7.62 (d, 2H), 7.80-7.85 (m, 2H), 8.00 (d, 1H), 8.05 (d, 1H), 8.18 (d, 2H), 8.99 (d, 1H), 9.43 (br t, 1H), 9.53 (d, 1H).

### Example 5: Preparation of 2-[4-[(imidazo[1,2-a]pyrimidin-6-carbonylamino)methyl]phenyl]-1,3-benzoxazol-6-carboxylic acid

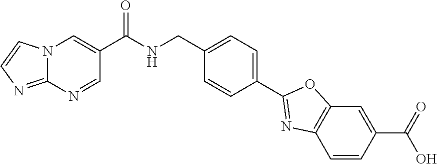 [SMILES: O=C(O)c1ccc2nc(-c3ccc(CNC(=O)c4cnc5nccn5c4)cc3)oc2c1]

The title compound was obtained through the processes of Steps A, B, and C.

### Step A: Preparation of methyl 2-[4-(aminomethyl)phenyl]-1,3-benzoxazol-6-carboxylate

The title compound (1.0 g, 21.4%) was obtained in the same manner as in Step A of Example 1 using 4-(aminomethyl)benzoic acid (2.5 g, 16.54 mmol) and methyl 4-amino-3-hydroxy-benzoate (2.76 g, 16.54 mmol).

¹H NMR (400 MHz, CDCl₃) δ ppm 3.90 (s, 3H), 3.91 (s, 2H), 7.43 (d, 2H), 7.70 (d, 1H), 8.02 (m, 1H), 8.15-8.22 (m, 3H).

### Step B: Preparation of methyl 2-[4-[(imidazo[1,2-a]pyrimidin-6-carbonylamino)methyl]phenyl]-1,3-benzoxazol-6-carboxylate

The title compound (340 mg, 28.6%) was obtained in the same manner as in Step B of Example 2 using imidazo[1,2-a]pyrimidin-6-carboxylic acid (525.88 mg, 3.22 mmol), oxalyl chloride (2.17 mL, 24.80 mmol), dimethyl formamide (1.91 μL, 24.80 μmol), triethylamine (3.45 mL, 24.80 mmol), and the methyl 2-[4-(aminomethyl)phenyl]-1,3-benzoxazol-6-carboxylate (700 mg, 2.48 mmol) obtained in Step A.

MS [M+H]=428 (M+1)

### Step C: Preparation of 2-[4-[(imidazo[1,2-a]pyrimidin-6-carbonylamino)methyl]phenyl]-1,3-benzoxazol-6-carboxylic acid

The methyl 2-[4-[(imidazo[1,2-a]pyrimidin-6-carbonylamino)methyl]phenyl]-1,3-benzoxazol-6-carboxylate (210 mg, 491.33 μmol) obtained in Step B was dissolved in pyridine (5 mL), lithium iodide (197.29 mg, 1.47 mmol) was added, and the resulting solution was then stirred at 120° C. for 40 hours. The reaction solution was concentrated under reduced pressure and purified by prep-HPLC to obtain the title compound (11.6 mg, 5.5%).

¹H NMR (400 MHz, DMSO-d₆) δ ppm 4.66 (d, 2H), 7.64 (d, 2H), 7.83 (d, 1H), 7.88 (d, 1H), 8.00-8.07 (m, 2H), 8.23 (d, 2H), 8.28 (d, 1H), 9.00 (d, 1H), 9.44 (t, 1H), 9.55 (d, 1H).

### Example 6: Preparation of N-[[4-(5-methyl-1,3-benzoxazol-2-yl)phenyl]methyl]imidazo[1,2-a]pyrimidin-6-carboxamide

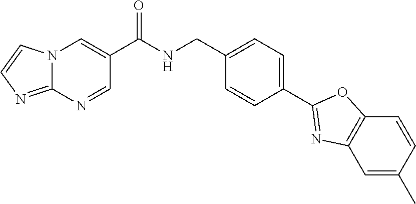 [SMILES: Cc1ccc2oc(-c3ccc(CNC(=O)c4cnc5nccn5c4)cc3)nc2c1]

The title compounds were obtained through the processes of Steps A and B below.

### Step A: Preparation of [4-(5-methyl-1,3-benzoxazol-2-yl)phenyl]methanamine

The title compound (1.23 g, 78.0%) was obtained in the same manner as in Step A of Example 1 using 4-(aminomethyl)benzoic acid (1.0 g, 6.62 mmol) and 2-amino-4-methyl-phenol (814.7 mg, 6.62 mmol).

¹H NMR (400 MHz, CDCl₃) δ ppm 2.41 (s, 3H), 3.89 (s, 2H), 7.08 (m, 1H), 7.39 (m, 3H), 7.47 (d, 1H), 8.14 (d, 2H).

### Step B: Preparation of N-[[4-(5-methyl-1,3-benzoxazol-2-yl)phenyl]methyl]imidazo[1,2-a]pyrimidin-6-carboxamide

The title compound (13.9 mg, 5.8%) was obtained in the same manner as in Step B of Example 2 using imidazole[1,2-a]pyrimidin-6-carboxylic acid (200 mg, 1.23 mmol), oxalyl chloride (536.85 μL, 6.13 mmol), dimethyl formamide (4.72e-1 μL, 6.13 μmol), triethylamine (853.21 μL, 6.13 mmol), and the [4-(5-methyl-1,3-benzoxazol-2-yl)phenyl]methanamine (146.07 mg, 612.99 μmol) obtained in Step A.

¹H NMR (400 MHz, DMSO-d₆) δ ppm 2.45 (s, 3H), 4.64 (d, 2H), 7.25 (m, 1H), 7.57-7.63 (m, 3H), 7.66 (d, 1H), 7.83 (d, 1H), 8.05 (d, 1H), 8.17 (d, 2H), 8.99 (d, 1H), 9.42 (br t, 1H), 9.53 (d, 1H).

### Example 7: Preparation of N-[[4-(5-methoxy-1,3-benzoxazol-2-yl)phenyl]methyl]imidazo[1,2-a]pyrimidin-6-carboxamide

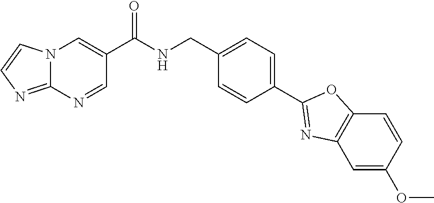 [SMILES: COc1ccc2oc(-c3ccc(CNC(=O)c4cnc5nccn5c4)cc3)nc2c1]

The title compounds were obtained through the processes of Steps A and B below.

### Step A: Preparation of [4-(5-methoxy-1,3-benzoxazol-2-yl)phenyl]methanamine

The title compound (400 mg, 23.8%) was obtained in the same manner as in Step A of Example 1 using 4-(aminomethyl)benzoic acid (1.0 g, 6.62 mmol) and 2-amino-4-methoxy-phenol (920.54 mg, 6.62 mmol).

¹H NMR (400 MHz, CDCl₃) δ ppm 8.21 (d, 2H), 7.48 (t, 3H), 7.26 (s, 1H), 6.95 (dd, 1H), 3.98 (s, 2H), 3.88 (s, 3H).

### Step B: Preparation of N-[[4-(5-methoxy-1,3-benzoxazol-2-yl)phenyl]methyl]imidazo[1,2-a]pyrimidin-6-carboxamide

The title compound (51.0 mg, 27.8%) was obtained in the same manner as in Step B of Example 2 using imidazole[1,2-a]pyrimidin-6-carboxylic acid (150 mg, 919.49 μmol), oxalyl chloride (643.92 μL, 7.36 mmol), dimethyl formamide (3.54 μL, 45.97 μmol), triethylamine (1.2 mL, 9.19 mmol), and the [4-(5-methyl-1,3-benzoxazol-2-yl)phenyl]methanamine (116.91 mg, 459.75 μmol) obtained in Step A.

¹H NMR (400 MHz, DMSO-d₆) δ ppm 9.54 (br s, 1H), 9.42 (br s, 1H), 8.99 (br s, 1H), 8.16 (br d, 2H), 8.05 (s, 1H), 7.83 (s, 1H), 7.68 (br d, 1H), 7.60 (br d, 2H), 7.35 (br s, 1H), 7.01 (br d, 1H), 4.64 (br d, 2H), 3.83 (s, 3H).

### Example 8: Preparation of N-[[4-(6-methyl-1,3-benzothiazol-2-yl)phenyl]methyl]imidazo[1,2-a]pyrimidin-6-carboxamide

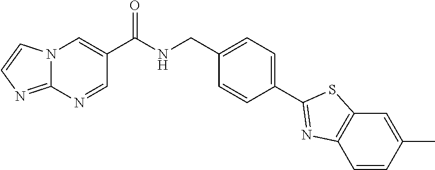 [SMILES: Cc1ccc2nc(-c3ccc(CNC(=O)c4cnc5nccn5c4)cc3)sc2c1]

The title compounds were obtained through the processes of Steps A and B below.

### Step A: Preparation of [4-(6-methyl-1,3-benzothiazol-2-yl)phenyl]methanamine

[4-(aminomethyl)phenyl]boronic acid (688.31 mg, 4.56 mmol), 2-bromo-6-methyl-1,3-benzothiazole (800 mg, 3.51 mmol), sodium carbonate (743.43 mg, 7.01 mmol), and [1,1′-bis(diphenylphosphino)ferrocene]dichloropalladium(II) were dissolved in dimethyl ether (10 mL) and water (3 mL), and the resulting solution was stirred at 80° C. for 12 hours. After lowering the reaction solution to room temperature, a 1M sodium hydroxide aqueous solution (30 mL) was added, and the reaction solution was extracted twice with ethyl acetate. After washing an organic layer with a sodium chloride aqueous solution, a filtrate dried and filtered with anhydrous sodium sulfate was concentrated under reduced pressure and purified through column chromatography to obtain the title compound (180 mg, 20.2%).

$$MS [ M + H ] = 255.1 ( M + 1 )$$

### Step B: Preparation of N-[[4-(6-methyl-1,3-benzothiazol-2-yl)phenyl]methyl]imidazo[1,2-a]pyrimidin-6-carboxamide

The title compound (32.0 mg, 13.1%) was obtained in the same manner as in Step B of Example 2 using imidazo[1,2-a]pyrimidin-6-carboxylic acid (200 mg, 1.23 mmol), oxalyl chloride (858.84 μL, 9.81 mmol), dimethyl formamide (4.72 μL, 61.30 μmol), triethylamine (1.71 mL, 12.26 mmol), and the [4-(6-methyl-1,3-benzothiazol-2-yl)phenyl]methanamine (155.92 mg, 613.0 μmol) obtained in Step A.

¹H NMR (400 MHz, DMSO-d₆) δ ppm 9.54 (d, 1H), 9.41 (t, 1H), 8.99 (d, 1H), 8.07 (s, 1H), 8.05 (s, 2H), 7.92-7.96 (m, 2H), 7.83 (d, 1H), 7.56 (d, 2H), 7.37 (d, 1H), 4.62 (d, 2H), 2.47 (s, 3H).

### Example 9: Preparation of N-[[4-(5-methylsulfonyl-1,3-benzoxazol-2-yl)phenyl]methyl]imidazo[1,2-a]pyrimidin-6-carboxamide

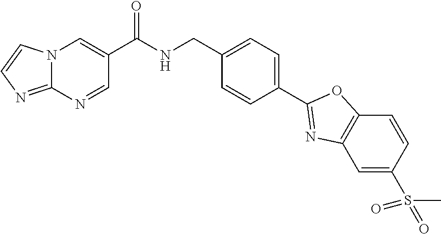 [SMILES: CS(=O)(=O)c1ccc2oc(-c3ccc(CNC(=O)c4cnc5nccn5c4)cc3)nc2c1]

The title compounds were obtained through the processes of Steps A and B below.

### Step A: Preparation of [4-(5-methylsulfonyl-1,3-benzoxazol-2-yl)phenyl]methanamine

The title compound (1.12 g, 56.0%) was obtained in the same manner as in Step A of Example 1 using 4-(aminomethyl)benzoic acid (1.0 g, 6.62 mmol), 2-amino-4-methylsulfonyl-phenol (1.24 g, 6.62 mmol).

¹H NMR (400 MHz, CDCl₃) δ ppm 3.05 (s, 3H), 3.93 (s, 2H), 7.45 (d, 2H), 7.67 (d, 1H), 7.90 (m, 1H), 8.17 (d, 2H), 8.28 (d, 1H).

### Step B: Preparation of N-[[4-(5-methylsulfonyl-1,3-benzoxazol-2-yl)phenyl]methyl]imidazo[1,2-a]pyrimidin-6-carboxamide

The title compound (8.1 mg, 5.4%) was obtained in the same manner as in Step B of Example 2 using imidazo[1,2-a]pyrimidin-6-carboxylic acid (80.93 mg, 496.12 μmol), oxalyl chloride (213.26 μL, 2.32 mmol), dimethyl formamide (1.27 μL, 16.54 μmol), triethylamine (231.18 μL, 12.26 mmol), and the [4-(5-methylsulfonyl-1,3-benzoxazol-2-yl)phenyl]methanamine (100 mg, 330.74 μmol) obtained in Step A.

¹H NMR (400 MHz, DMSO-d₆) δ ppm 3.30 (s, 3H), 4.66 (br d, 2H), 7.65 (d, 2H), 7.83 (s, 1H), 7.96-8.11 (m, 3H), 8.24 (d, 2H), 8.35 (s, 1H), 8.99 (d, 1H), 9.44 (br t, 1H), 9.53 (d, 1H).

### Example 10: Preparation of N-[[4-(6-chloro-5-methyl-1,3-benzoxazol-2-yl)phenyl]methyl]imidazo[1,2-a]pyrimidin-6-carboxamide

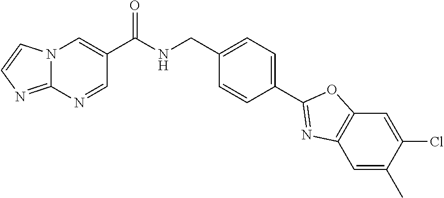 [SMILES: Cc1cc2nc(-c3ccc(CNC(=O)c4cnc5nccn5c4)cc3)oc2cc1Cl]

The title compounds were obtained through the processes of Steps A and B below.

### Step A: Preparation of [4-(6-chloro-5-methyl-1,3-benzoxazol-2-yl)phenyl]methanamine

The title compound (640 mg, 73.9%) was obtained in the same manner as in Step A of Example 1 using 4-(aminomethyl)benzoic acid (479.58 mg, 3.17 mmol) and the 2-amino-5-chloro-4-methyl-phenol (500 mg, 3.17 mmol) obtained in Preparation Example 2.

¹H NMR (400 MHz, CDCl₃) δ ppm 2.41 (s, 3H), 3.90 (s, 2H), 7.41 (d, 2H), 7.52 (d, 2H), 8.11 (d, 2H).

### Step B: Preparation of N-[[4-(6-chloro-5-methyl-1,3-benzoxazol-2-yl)phenyl]methyl]imidazo[1,2-a]pyrimidin-6-carboxamide

The title compound (40.7 mg, 17.4%) was obtained in the same manner as in Step B of Example 2 using imidazole[1,2-a]pyrimidin-6-carboxylic acid (179.45 mg, 1.10 mmol), oxalyl chloride (506.60 μL, 5.50 mmol), dimethyl formamide (4.23e-1 μL, 5.50 μmol), triethylamine (765.52 μL, 5.50 mmol), and the [4-(6-chloro-5-methyl-1,3-benzoxazol-2-yl)phenyl]methanamine (150 mg, 550.0 μmol) obtained in Step A.

¹H NMR (400 MHz, DMSO-d₆) δ ppm 2.45 (br s, 3H), 4.64 (br d, 2H), 7.61 (br d, 2H), 7.82 (br d, 2H), 7.96 (br d, 1H), 8.05 (br s, 1H), 8.12-8.21 (m, 2H), 8.99 (br s, 1H), 9.39-9.46 (m, 1H), 9.53 (br s, 1H).

### Example 11: Preparation of N-[[4-(6-fluoro-5-methyl-1,3-benzoxazol-2-yl)phenyl]methyl]imidazo[1,2-a]pyrimidin-6-carboxamide

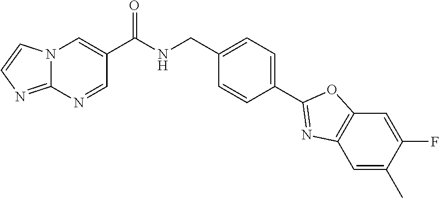 [SMILES: Cc1cc2nc(-c3ccc(CNC(=O)c4cnc5nccn5c4)cc3)oc2cc1F]

The title compounds were obtained through the processes of Steps A and B below.

### Step A: Preparation of [4-(6-fluoro-5-methyl-1,3-benzoxazol-2-yl)phenyl]methanamine

The title compound (650 mg, 70.4%) was obtained in the same manner as in Step A of Example 1 using 4-(aminomethyl)benzoic acid (545 mg, 3.61 mmol), and the 2-amino-5-fluoro-4-methyl-phenol (508.88 mg, 3.61 mmol) obtained in Preparation Example 3.

¹H NMR (400 MHz, CDCl₃) δ ppm 8.20 (d, 2H), 7.56 (d, 1H), 7.50 (d, 2H), 7.29 (s, 1H), 3.99 (s, 2H), 2.39-2.44 (m, 1H), 2.41 (d, 2H).

### Step B: Preparation of N-[[4-(6-fluoro-5-methyl-1,3-benzoxazol-2-yl)phenyl]methyl]imidazo[1,2-a]pyrimidin-6-carboxamide

The title compound (11.5 mg, 6.2%) was obtained in the same manner as in Step B of Example 2 using imidazole[1,2-a]pyrimidin-6-carboxylic acid (150 mg, 919.49 μmol), oxalyl chloride (643.92 μL, 7.36 mmol), dimethyl formamide (3.54 μL, 45.97 μmol), triethylamine (1.28 mL, 9.19 mmol), and the [4-(6-fluoro-5-methyl-1,3-benzoxazol-2-yl)phenyl]methanamine (117.82 mg, 459.75 μmol) obtained in Step A.

¹H NMR (400 MHz, DMSO-d₆) δ ppm 9.54 (s, 1H), 9.43 (s, 1H), 8.99 (s, 1H), 8.15 (d, 2H), 8.05 (s, 1H), 7.83 (s, 1H), 7.71-7.77 (m, 2H), 7.60 (d, 2H), 4.64 (d, 2H), 2.35 (s, 3H).

### Example 12: Preparation of N-[[4-(5-methylsulfonyl-1,3-benzoxazol-2-yl)phenyl]methyl]imidazo[1,2-a]pyridine-6-carboxamide

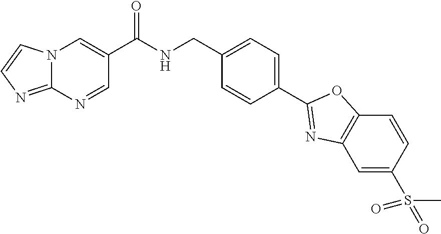 [SMILES: CS(=O)(=O)c1ccc2oc(-c3ccc(CNC(=O)c4cnc5nccn5c4)cc3)nc2c1]

The title compound was obtained through the process of Step A below.

### Step A: Preparation of N-[[4-(5-methylsulfonyl-1,3-benzoxazol-2-yl)phenyl]methyl]imidazo[1,2-a]pyridin-6-carboxamide

The title compound (8.8 mg, 1.9%) was obtained in the same manner as in Step B of Example 1 using the [4-(5-methylsulfonyl-1,3-benzoxazol-2-yl)phenyl]methanamine (300 mg, 992.24 μmol) obtained in Step A of Example 9, imidazo[1,2-a]pyrimidin-6-carboxylic acid (160.89 mg, 992.24 μmol), and 1-ethyl-3-(3-dimethylaminopropyl)carbodiimide (285.32 mg, 1.49 mmol).

¹H NMR (400 MHz, DMSO-d₆) δ ppm 3.30 (s, 3H), 4.64 (d, 2H), 7.60-7.68 (m, 4H), 7.69-7.74 (m, 1H), 7.98-8.03 (m, 1H), 8.05-8.12 (m, 2H), 8.24 (d, 2H), 8.36 (d, 1H), 9.20 (s, 1H), 9.28 (t, 1H).

### Example 13: Preparation of N-[[4-[5-(trifluoromethyl)-1,3-benzoxazol-2-yl]phenyl]methyl]imidazo[1,2-a]pyrimidin-6-carboxamide

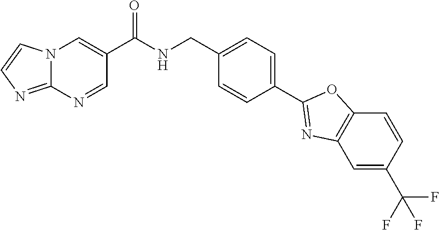 [SMILES: O=C(NCc1ccc(-c2nc3cc(C(F)(F)F)ccc3o2)cc1)c1cnc2nccn2c1]

The title compounds were obtained through the processes of Steps A and B below.

### Step A: Preparation of [4-[5-(trifluoromethyl)-1,3-benzoxazol-2-yl]phenyl]methanamine

The title compound (0.39 g, 20.2%) was obtained in the same manner as in Step A of Example 1 using 4-(aminomethyl)benzoic acid (1.0 g, 6.62 mmol) and 2-amino-4-(trifluoromethyl)phenol (1.17 g, 6.62 mmol).

¹H NMR (400 MHz, CDCl₃) δ ppm 8.17 (d, 2H), 7.97 (s, 1H), 7.54-7.64 (m, 2H), 7.45 (d, 2H), 3.93 (s, 2H).

### Step B: Preparation of N-[[4-[5-(trifluoromethyl)-1,3-benzoxazol-2-yl]phenyl]methyl]imidazo[1,2-a]pyrimidin-6-carboxamide

The title compound (60.5 mg, 11.3%) was obtained in the same manner as in Step B of Example 2 using imidazole[1,2-a]pyrimidin-6-carboxylic acid (200 mg, 1.23 mmol), oxalyl chloride (858.57 μL, 9.81 mmol), dimethyl formamide (4.72 μL, 61.30 μmol), triethylamine (1.71 mL, 12.26 mmol), and the [4-[5-(trifluoromethyl)-1,3-benzoxazol-2-yl]phenyl]methanamine (179.15 mg, 613.0 μmol) obtained in Step A.

¹H NMR (400 MHz, DMSO-d₆) δ ppm 9.55 (d, 1H), 9.43 (t, 1H), 9.00 (d, 1H), 8.23 (d, 3H), 8.05 (s, 2H), 7.78-7.85 (m, 2H), 7.64 (d, 2H), 4.66 (d, 2H).

### Example 14: Preparation of N-[[4-(6-ethylsulfonyl-1,3-benzoxazol-2-yl)phenyl]methyl]imidazo[1,2-a]pyrimidin-6-carboxamide

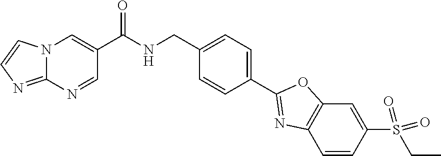 [SMILES: CCS(=O)(=O)c1ccc2nc(-c3ccc(CNC(=O)c4cnc5nccn5c4)cc3)oc2c1]

The title compounds were obtained through the processes of Steps A and B below.

### Step A: Preparation of [4-(6-ethylsulfonyl-1,3-benzoxazol-2-yl)phenyl]methanamine

The title compound (190 mg, 30.2%) was obtained in the same manner as in Step a of Example 1 using 4-(aminomethyl)benzoic acid (300.46 mg, 1.99 mmol) and the 2-amino-5-ethylsulfonyl-phenol (400 mg, 1.99 mmol) obtained in Preparation Example 4.

¹H NMR (400 MHz, DMSO-d₆) δ ppm 1.10-1.16 (m, 3H), 3.17 (q, 2H), 3.88 (br d, 2H), 7.11-7.21 (m, 1H), 7.54 (br d, 1H), 7.64 (d, 1H), 7.89-7.96 (m, 2H), 8.23 (t, 2H).

### Step B: Preparation of N-[[4-(6-ethylsulfonyl-1,3-benzoxazol-2-yl)phenyl]methyl]imidazo[1,2-a]pyrimidin-6-carboxamide

The title compound (11.0 mg, 3.9%) was obtained in the same manner as in Step B of Example 2 using imidazole[1,2-a]pyrimidin-6-carboxylic acid (200 mg, 1.23 mmol), oxalyl chloride (861.37 μL, 9.84 mmol), dimethyl formamide (4.73 μL, 61.50 μmol), triethylamine (1.71 mL, 12.30 mmol), and the [4-(6-ethylsulfonyl-1,3-benzoxazol-2-yl)phenyl]methanamine (194.57 mg, 615.0 μmol) obtained in Step A.

¹H NMR (400 MHz, DMSO-d₆) δ ppm 9.54 (d, 1H), 9.44 (t, 1H), 9.00 (d, 1H), 8.34 (d, 1H), 8.25 (d, 2H), 8.03-8.09 (m, 2H), 7.93 (dd, 1H), 7.83 (d, 1H), 7.66 (d, 2H), 4.67 (d, 2H), 3.37-3.41 (m, 2H), 1.13 (t, 3H).

### Example 15: Preparation of N-[[4-[5-(propanoylamino)-1,3-benzoxazol-2-yl]phenyl]methyl]imidazo[1,2-a]pyrimidin-6-carboxamide

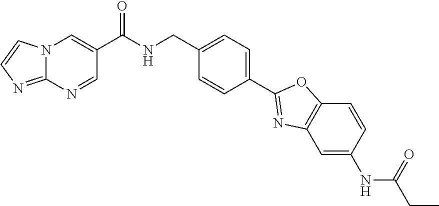 [SMILES: CCC(=O)Nc1ccc2oc(-c3ccc(CNC(=O)c4cnc5nccn5c4)cc3)nc2c1]

The title compound was obtained through the processes of Steps A, B, and C.

### Step A: Preparation of [4-(5-bromo-1,3-benzoxazol-2-yl)phenyl]methanamine

The title compound (4.75 g, 92.7%) was obtained in the same manner as in Step A of Example 1 using 4-(aminomethyl)benzoic acid (2.56 g, 16.91 mmol) and 2-amino-4-bromo-phenol (3.18 g, 16.91 mmol). [M+H]=304 (M+1)

### Step B: Preparation of N-[[4-(5-bromo-1,3-benzoxazol-2-yl)phenyl]methyl]imidazo[1,2-a]pyrimidin-6-carboxamide

The title compound (390 mg, 53.9%) was obtained in the same manner as in Step B of Example 2 using imidazole[1,2-a]pyrimidin-6-carboxylic acid (400 mg, 2.45 mmol), oxalyl chloride (1.72 mL, 19.62 mmol), dimethyl formamide (9.43 μL, 122.60 μmol), triethylamine (1.71 mL, 12.26 mmol), and the [4-(5-bromo-1,3-benzoxazol-2-yl)phenyl]methanamine (371.66 mg, 1.23 mmol) obtained in Step A.

¹H NMR (400 MHz, DMSO-d₆) δ ppm 9.53 (d, 1H), 9.41 (t, 1H), 8.99 (d, 1H), 8.19 (d, 2H), 8.03-8.06 (m, 2H), 7.77-7.83 (m, 2H), 7.62 (d, 2H), 7.58 (d, 1H), 4.65 (d, 2H).

### Step C: Preparation of N-[[4-[5-(propanoylamino)-1,3-benzoxazol-2-yl]phenyl]methyl]imidazo[1,2-a]pyrimidin-6-carboxamide

After dissolving the N-[[4-(5-bromo-1,3-benzoxazol-2-yl)phenyl]methyl]imidazo[1,2-a]pyrimidin-6-carboxamide (0.29 g, 491.67 μmol) obtained in Step B in 1,4-dioxane (5 mL), propanamide (107.81 mg, 1.47 mmol), tris(dibenzylideneacetone)dipalladium(0) (22.51 mg, 24.58 μmol), Xantphos (28.45 mg, 49.17 μmol), and potassium phosphate tribasic (313.09 mg, 1.47 mmol) were sequentially added, and the resulting solution was stirred at 100° C. for 48 hours. The reaction solution was concentrated under reduced pressure to obtain a residue, which was purified by prep-HPLC to obtain the title compound (3.65 mg, 1.7%).

¹H NMR (400 MHz, DMSO-d₆) δ ppm 10.06 (s, 1H), 9.55 (d, 1H), 9.43 (s, 1H), 9.00 (d, 1H), 8.14-8.21 (m, 3H), 8.05 (s, 1H), 7.83 (s, 1H), 7.71 (d, 1H), 7.60 (d, 2H), 7.53 (d, 1H), 4.65 (d, 2H), 2.36 (q, 2H), 1.11 (t, 3H).

### Example 16: Preparation of N-[[4-(5-chloro-1,3-benzoxazol-2-yl)phenyl]methyl]imidazo[1,2-a]pyrimidin-6-carboxamide

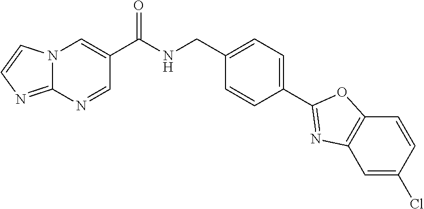 [SMILES: O=C(NCc1ccc(-c2nc3cc(Cl)ccc3o2)cc1)c1cnc2nccn2c1]

### Step A: Preparation of [4-(5-chloro-1,3-benzoxazol-2-yl)phenyl]methanamine

The title compound (2.9 g, 84.7%) was obtained in the same manner as in Step A of Example 1 using 4-(aminomethyl)benzoic acid (2.0 g, 13.23 mmol) and 2-amino-4-chloro-phenol (1.9 g, 13.23 mmol).

$$MS [ M + H ] = 259 ( M + 1 )$$

### Step B: Preparation of N-[[4-(5-chloro-1,3-benzoxazol-2-yl)phenyl]methyl]imidazo[1,2-a]pyrimidin-6-carboxamide

The title compound (49.8 mg, 21.1%) was obtained in the same manner as in Step B of Example 2 using imidazole[1,2-a]pyrimidin-6-carboxylic acid (200 mg, 1.23 mmol), oxalyl chloride (858.57 μL, 9.81 mmol), dimethyl formamide (4.72 μL, 61.30 μmol), triethylamine (853.22 μL, 6.13 mmol), and the [4-(5-chloro-1,3-benzoxazol-2-yl)phenyl]methanamine (158.58 mg, 613.00 μmol) obtained in Step A.

¹H NMR (400 MHz, DMSO-d₆) δ ppm 9.54 (d, 1H), 9.43 (t, 1H), 9.00 (d, 1H), 8.18 (d, 2H), 8.05 (d, 1H), 7.93 (d, 1H), 7.85 (d, 2H), 7.63 (d, 2H), 7.49 (m, 1H), 4.66 (d, 2H).

### Example 17: Preparation of N-[[4-(6-fluoro-1,3-benzoxazol-2-yl)phenyl]methyl]imidazo[1,2-a]pyrimidin-6-carboxamide

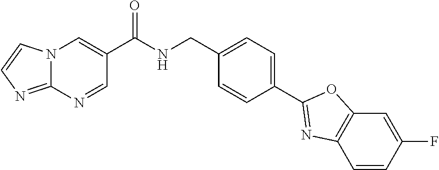 [SMILES: O=C(NCc1ccc(-c2nc3ccc(F)cc3o2)cc1)c1cnc2nccn2c1]

### Step A: Preparation of [4-(6-fluoro-1,3-benzoxazol-2-yl)phenyl]methanamine

The title compound (2.49 g, 77.7%) was obtained in the same manner as in Step A of Example 1 using 4-(aminomethyl)benzoic acid (2.0 g, 13.23 mmol) and 2-amino-5-fluoro-phenol (1.68 g, 13.23 mmol).

¹H NMR (400 MHz, DMSO-d₆) δ ppm 8.16 (d, 2H), 7.81-7.86 (m, 2H), 7.63 (d, 2H), 7.25 (d, 1H), 3.86 (s, 2H).

### Step B: Preparation of N-[[4-(6-fluoro-1,3-benzoxazol-2-yl)phenyl]methyl]imidazo[1,2-a]pyrimidin-6-carboxamide

The title compound (30.0 mg, 12.6%) was obtained in the same manner as in Step B of Example 2 using imidazole[1,2-a]pyrimidin-6-carboxylic acid (200 mg, 1.23 mmol), oxalyl chloride (858.57 μL, 9.81 mmol), dimethyl formamide (4.72 μL, 61.30 μmol), triethylamine (853.22 μL, 6.13 mmol), and the [4-(6-fluoro-1,3-benzoxazol-2-yl)phenyl]methanamine (148.5 mg, 613.00 μmol) obtained in Step A.

¹H NMR (400 MHz, DMSO-d₆) δ ppm 9.56 (d, 1H), 9.43 (t, 1H), 9.02 (d, 1H), 8.18 (d, 2H), 8.05 (d, 1H), 7.70-7.90 (m, 3H), 7.62 (d, 2H), 7.33 (m, 1H), 4.65 (d, 2H).

### Example 18: Preparation of N-[[4-(5-fluoro-1,3-benzoxazol-2-yl)phenyl]methyl]imidazo[1,2-a]pyrimidin-6-carboxamide

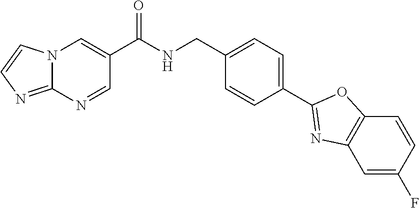 [SMILES: O=C(NCc1ccc(-c2nc3cc(F)ccc3o2)cc1)c1cnc2nccn2c1]

The title compounds were obtained through the processes of Steps A and B below.

### Step A: Preparation of [4-(5-fluoro-1,3-benzoxazol-2-yl)phenyl]methanamine

The title compound (1.7 g, 53.0%) was obtained in the same manner as in Step A of Example 1 using 4-(aminomethyl)benzoic acid (2.0 g, 13.23 mmol) and 2-amino-4-fluoro-phenol (1.68 g, 13.23 mmol).

¹H NMR (400 MHz, DMSO-d₆) δ ppm 8.20 (d, 2H), 7.45-7.52 (m, 3H), 7.43 (d, 1H), 7.10 (d, 1H), 3.98 (d, 2H).

### Step B: Preparation of N-[[4-(5-fluoro-1,3-benzoxazol-2-yl)phenyl]methyl]imidazo[1,2-a]pyrimidin-6-carboxamide

The title compound (12.7 mg, 2.7%) was obtained in the same manner as in Step B of Example 2 using imidazole[1,2-a]pyrimidin-6-carboxylic acid (200 mg, 1.23 mmol), oxalyl chloride (861.37 μL, 9.84 mmol), dimethyl formamide (4.73 μL, 61.50 μmol), triethylamine (856.01 μL, 6.15 mmol), and the [4-(5-fluoro-1,3-benzoxazol-2-yl)phenyl]methanamine (148.98 mg, 615.0 μmol) obtained in Step A.

¹H NMR (400 MHz, DMSO-d₆) δ ppm 9.54 (d, 1H), 9.43 (t, 1H), 9.00 (d, 1H), 8.18 (d, 2H), 8.05 (d, 1H), 7.75-7.85 (m, 2H), 7.85 (d, 1H), 7.63 (d, 2H), 7.20-7.30 (m, 1H), 4.66 (d, 2H).

### Example 19: Preparation of N-[[4-[6-(trifluoromethyl)-1,3-benzoxazol-2-yl]phenyl]methyl]imidazo[1,2-a]pyrimidin-6-carboxamide

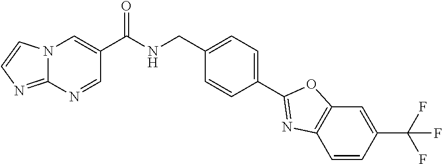 [SMILES: O=C(NCc1ccc(-c2nc3ccc(C(F)(F)F)cc3o2)cc1)c1cnc2nccn2c1]

The title compounds were obtained through the processes of Steps A and B below.

### Step A: Preparation of [4-[6-(trifluoromethyl)-1,3-benzoxazol-2-yl]phenyl]methanamine

The title compound (220 mg, 14.2%) was obtained in the same manner as in Step a of Example 1 using 4-(aminomethyl)benzoic acid (800 mg, 5.29 mmol) [symbol] 2-amino-5-(trifluoromethyl)phenol (936.98 mg, 5.29 mmol).

¹H NMR (400 MHz, CDCl₃) δ ppm 8.17 (d, 2H), 7.74-7.82 (m, 2H), 7.57 (d, 1H), 7.45 (d, 2H), 3.94 (br, 2H).

### Step B: Preparation of N-[[4-[6-(trifluoromethyl)-1,3-benzoxazol-2-yl]phenyl]methyl]imidazo[1,2-a]pyrimidin-6-carboxamide

The title compound (139.3 mg, 21.2%) was obtained in the same manner as in Step B of Example 2 using imidazole[1,2-a]pyrimidin-6-carboxylic acid (245 mg, 1.50 mmol), oxalyl chloride (1.05 mL, 12.01 mmol), dimethyl formamide (5.78 μL, 75.09 μmol), triethylamine (1.05 mL, 7.51 mmol), and the [4-[6-(trifluoromethyl)-1,3-benzoxazol-2-yl]phenyl]methanamine (219.46 mg, 750.92 μmol) obtained in Step A.

¹H NMR (400 MHz, DMSO-d₆) δ ppm 9.66 (d, 1H), 9.50 (t, 1H), 9.10 (d, 1H), 8.25 (d, 1H), 8.20 (d, 2H), 8.15 (d, 1H), 7.90-8.00 (d, 1H), 8.00-8.05 (d, 1H), 7.70 (d, 1H), 7.65 (d, 2H), 4.65 (d, 2H).

### Example 20: Preparation of N-[[4-(4-methyl-1,3-benzoxazol-2-yl)phenyl]methyl]imidazo[1,2-a]pyrimidin-6-carboxamide

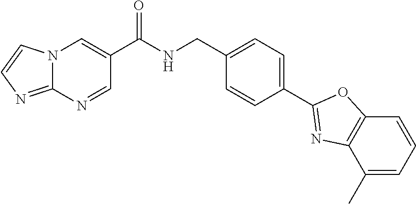 [SMILES: Cc1cccc2oc(-c3ccc(CNC(=O)c4cnc5nccn5c4)cc3)nc12]

The title compounds were obtained through the processes of Steps A and B below.

### Step A: Preparation of [4-(4-methyl-1,3-benzoxazol-2-yl)phenyl]methanamine

The title compound (2.35 g, 52.2%) was obtained in the same manner as in Step A of Example 1 using 4-(aminomethyl)benzoic acid (2.0 g, 13.23 mmol) and 2-amino-3-methyl-phenol (1.63 g, 13.23 mmol).

MS [M+H]=239 (M+1)

$$MS [ M + H ] = 239 ( M + 1 )$$

### Step B: Preparation of N-[[4-(4-methyl-1,3-benzoxazol-2-yl)phenyl]methyl]imidazo[1,2-a]pyrimidin-6-carboxamide

The title compound (75.7 mg, 16.1%) was obtained in the same manner as in Step B of Example 2 using imidazole[1,2-a]pyrimidin-6-carboxylic acid (200 mg, 1.23 mmol), oxalyl chloride (861.37 μL, 9.84 mmol), dimethyl formamide (4.73 μL, 61.50 μmol), triethylamine (856.01 μL, 6.15 mmol), and the [4-(4-methyl-1,3-benzoxazol-2-yl)phenyl]methanamine (209.35 mg, 615.0 mol, purity: 70%) obtained in Step A.

¹H NMR (400 MHz, DMSO-d₆) δ ppm 9.60 (d, 1H), 9.45 (t, 1H), 9.00 (d, 1H), 8.20 (d, 2H), 8.05 (d, 1H), 7.83 (d, 1H), 7.52-7.60 (m, 3H), 7.35 (t, 1H), 7.25 (d, 1H), 4.65 (d, 2H), 2.50 (s, 3H).

### Example 21: Preparation of N-[[4-(7-methyl-1,3-benzoxazol-2-yl)phenyl]methyl]imidazo[1,2-a]pyrimidin-6-carboxamide

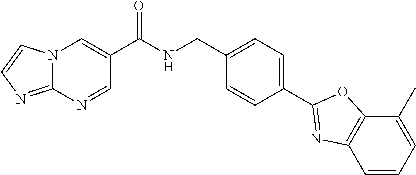 [SMILES: Cc1cccc2nc(-c3ccc(CNC(=O)c4cnc5nccn5c4)cc3)oc12]

The title compounds were obtained through the processes of Steps A and B below.

### Step A: Preparation of [4-(7-methyl-1,3-benzoxazol-2-yl)phenyl]methanamine

The title compound (1.3 g, 82.5%) was obtained in the same manner as in Step A of Example 1 using 4-(aminomethyl)benzoic acid (1.0 g, 6.62 mmol) and 2-amino-6-methyl-phenol (814.7 mg, 6.62 mmol).

$$MS [ M + H ] = 239 ( M + 1 )$$

### Step B: Preparation of N-[[4-(7-methyl-1,3-benzoxazol-2-yl)phenyl]methyl]imidazo[1,2-a]pyrimidin-6-carboxamide

The title compound (78.3 mg, 16.6%) was obtained in the same manner as in Step B of Example 2 using imidazole[1,2-a]pyrimidin-6-carboxylic acid (200 mg, 1.23 mmol), oxalyl chloride (861.37 μL, 9.84 mmol), dimethyl formamide (4.73 μL, 61.50 μmol), triethylamine (856.01 μL, 6.15 mmol), and the [4-(7-methyl-1,3-benzoxazol-2-yl)phenyl]methanamine (146.54 mg, 615.0 μmol) obtained in Step A.

¹H NMR (400 MHz, DMSO-d₆) δ ppm 9.55 (d, 1H), 9.45 (t, 1H), 9.00 (d, 1H), 8.20 (d, J=8.00 Hz, 2H), 8.05 (d, 1H), 7.83 (d, 1H), 7.60 (d, 3H), 7.35 (t, 1H), 7.25 (d, 1H), 4.65 (d, 2H), 2.50 (s, 3H).

### Example 22: Preparation of N-[[4-(6-methoxy-1,3-benzoxazol-2-yl)phenyl]methyl]imidazo[1,2-a]pyrimidin-6-carboxamide

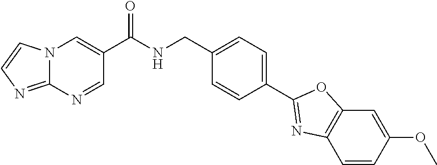 [SMILES: COc1ccc2nc(-c3ccc(CNC(=O)c4cnc5nccn5c4)cc3)oc2c1]

The title compounds were obtained through the processes of Steps A and B below.

### Step A: Preparation of [4-(6-methoxy-1,3-benzoxazol-2-yl)phenyl]methanamine

The title compound (0.58 g, 28.7%) was obtained in the same manner as in Step A of Example 1 using 4-(aminomethyl)benzoic acid (1.2 g, 7.94 mmol) and 2-amino-5-methoxy-phenol (1.1 g, 7.94 mmol).

$$MS [ M + H ] = 255 ( M + 1 )$$

### Step B: Preparation of N-[[4-(6-methoxy-1,3-benzoxazol-2-yl)phenyl]methyl]imidazo[1,2-a]pyrimidin-6-carboxamide

The title compound (117.4 mg, 23.9%) was obtained in the same manner as in Step B of Example 2 using imidazole[1,2-a]pyrimidin-6-carboxylic acid (200.65 mg, 1.23 mmol), oxalyl chloride (861.37 μL, 9.84 mmol), dimethyl formamide (4.73 μL, 61.50 μmol), triethylamine (856.00 μL, 6.15 mmol), and the [4-(6-methoxy-1,3-benzoxazol-2-yl)phenyl]methanamine (156.38 mg, 615.0 μmol) obtained in Step A.

¹H NMR (400 MHz, DMSO-d₆) δ ppm 9.54 (s, 1H), 9.42 (s, 1H), 8.99 (s, 1H), 8.16 (d, 2H), 8.05 (s, 1H), 7.83 (s, 1H), 7.68 (d, 1H), 7.55 (d, 2H), 7.40 (s, 1H), 7.00 (d, 1H), 4.60 (d, 2H), 3.85 (s, 3H).

### Example 23: Preparation of N-[[4-(5-hydroxy-1,3-benzoxazol-2-yl)phenyl]methyl]imidazo[1,2-a]pyrimidin-6-carboxamide

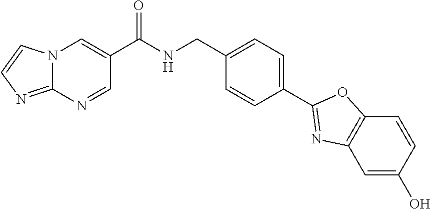 [SMILES: O=C(NCc1ccc(-c2nc3cc(O)ccc3o2)cc1)c1cnc2nccn2c1]

The title compounds were obtained through the processes of Steps A and B below.

### Step A: Preparation of N-[[4-[5-(4,4,5,5-tetramethyl-1,3,2-dioxabororan-2-yl)-1,3-benzoxazol-2-yl]phenyl]methyl]imidazo[1,2-a]pyrimidin-6-carboxamide

The N-[[4-(5-bromo-1,3-benzoxazol-2-yl)phenyl]methyl]imidazo[1,2-a]pyrimidin-6-carboxamide (900 mg, 2.01 mmol) obtained in Step B of Example 15 was dissolved in 1,4-dioxane (15 mL), bis(pinacolato)diboron (765.62 mg, 3.01 mmol), [1,1′ bis(diphenylphosphino)ferrocene]dichloropalladium (II) (147.07 mg, 201.0 μmol), and potassium acetate (591.80 mg, 6.03 mmol) were sequentially added, and then the resulting solution as stirred at 100° C. for 3 hours. The reaction solution was concentrated under reduced pressure to obtain a residue, which was purified through column chromatography to obtain the title compound (350 mg, 35.2%).

$$MS [ M + H ] = 496 ( M + 1 )$$

### Step B: Preparation of N-[[4-(5-hydroxy-1,3-benzoxazol-2-yl)phenyl]methyl]imidazo[1,2-a]pyrimidin-6-carboxamide

The N-[[4-[5-(4,4,5,5-tetra methyl-1,3,2-dioxabororan-2-yl)-1,3-benzoxazol-2-yl]phenyl]methyl]imidazo[1,2-a]pyrimidin-6-carboxamide (350 mg, 706.59 μmol) obtained in Step A was dissolved in ethanol (6 mL) and water (3 mL), m-chloroperoxybenzoic acid (215.18 mg, purity: 85%, 1.06 mmol) was added, and then the resulting solution was stirred at room temperature for 12 hours. The reaction solution was concentrated under reduced pressure to obtain a residue, which was purified through prep-HPLC to obtain the title compound (16.0 mg, 5.9%).

¹H NMR (400 MHz, DMSO-d₆) δ ppm 9.5-9.6 (m, 2H), 9.45 (t, 1H), 9.05 (d, 1H), 8.20 (d, 2H), 8.10 (d, 1H), 7.95 (d, 1H), 7.5-7.6 (m, 3H), 7.10 (d, 1H), 6.85-6.92 (m, 1H), 4.65 (d, 2H).

### Example 24: Preparation of N-[[4-(5-methyloxazolo[5,4-b]pyridin-2-yl)phenyl]methyl]imidazo[1,2-a]pyrimidin-6-carboxamide

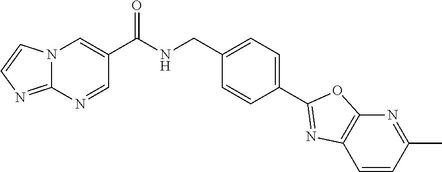 [SMILES: Cc1ccc2nc(-c3ccc(CNC(=O)c4cnc5nccn5c4)cc3)oc2n1]

The title compounds were obtained through the processes of Steps A and B below.

### Step A: Preparation of [4-(5-methyloxazolo[5,4-b]pyridin-2-yl)phenyl]methanamine

The title compound (160 mg, 41.5%) was obtained in the same manner as in Step A of Example 1 using 4-(aminomethyl)benzoic acid (243.53 mg, 1.61 mmol) and the 3-amino-6-methyl-pyridin-2-ol (200 mg, 1.61 mmol) obtained in Preparation Example 5.

¹H NMR (400 MHz, CDCl₃) δ ppm 8.23 (d, 2H), 7.94 (d, 1H), 7.51 (d, 2H), 7.21 (d, 1H), 3.99 (s, 2H), 2.69 (s, 3H).

### Step B: Preparation of N-[[4-(5-methyloxazolo[5,4-b]pyridin-2-yl)phenyl]methyl]imidazo[1,2-a]pyrimidin-6-carboxamide

The title compound (56.1 mg, 52.9%) was obtained in the same manner as in Step B of Example 2 using imidazole[1,2-a]pyrimidin-6-carboxylic acid (90 mg, 551.7 μmol), oxalyl chloride (386.34 μL, 4.41 mmol), triethylamine (383.94 μL, 2.76 mmol), and the [4-(5-methyloxazolo[5,4-b]pyridin-2-yl)phenyl]methanamine (66.0 mg, 275.85 μmol) obtained in Step A.

¹H NMR (400 MHz, DMSO-d₆) δ ppm 9.53 (d, 1H), 9.41 (t, 1H), 8.99 (d, 1H), 8.18 (d, 2H), 8.14 (d, 1H), 8.05 (s, 1H), 7.82 (s, 1H), 7.62 (d, 2H), 7.37 (d, 1H), 4.65 (d, 2H), 2.60 (s, 3H).

### Example 25: Preparation of N-[[4-[5-(2-pyridyloxy)-1,3-benzoxazol-2-yl]phenyl]methyl]imidazo[1,2-a]pyrimidin-6-carboxamide

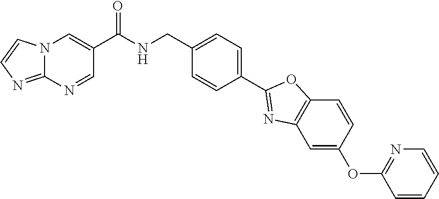 [SMILES: O=C(NCc1ccc(-c2nc3cc(Oc4ccccn4)ccc3o2)cc1)c1cnc2nccn2c1]

The title compounds were obtained through the processes of Steps A and B below.

### Step A: Preparation of [4-[5-(2-pyridyloxy)-1,3-benzoxazol-2-yl]phenyl]methanamine

The title compound (0.284 g, 90.4%) was obtained in the same manner as in Step A of Example 1 using 4-(aminomethyl)benzoic acid (149.51 mg, 13.23 mmol) and the 2-amino-4-(2-pyridyloxy)phenol (200 mg, 989.1 μmol) obtained in Preparation Example 6.

$$MS [ M + H ] = 318 ( M + 1 )$$

### Step B: Preparation of N-[[4-[5-(2-pyridyloxy)-1,3-benzoxazol-2-yl]phenyl]methyl]imidazo[1,2-a]pyrimidin-6-carboxamide

The title compound (59.5 mg, 40.8%) was obtained in the same manner as in Step B of Example 2 using imidazole[1,2-a]pyrimidin-6-carboxylic acid (102.81 mg, 630.24 μmol), oxalyl chloride (441.34 μL, 5.04 mmol), dimethyl formamide (2.42 μL, 31.51 μmol), triethylamine (438.60 μL, 3.15 mmol), and the [4-[5-(2-pyridyloxy)-1,3-benzoxazol-2-yl]phenyl]methanamine (100 mg, 315.12 μmol) obtained in Step A.

¹H NMR (400 MHz, DMSO-d₆) δ ppm 9.52-9.55 (m, 1H), 9.38-9.43 (m, 1H), 8.99 (d, 1H), 8.20-8.22 (m, 1H), 8.18-8.20 (m, 1H), 8.14 (dd, 1H), 8.04-8.05 (m, 1H), 7.87 (m, 1H), 7.80-7.83 (m, 2H), 7.62-7.64 (m, 1H), 7.61 (s, 1H), 7.59 (d, 1H), 7.20 (dd, 1H), 7.11-7.15 (m, 1H), 7.05-7.09 (m, 1H), 4.65 (d, 2H).

### Example 26: Preparation of N-[[4-(6-amino-1,3-benzoxazol-2-yl)phenyl]methyl]imidazo[1,2-a]pyrimidin-6-carboxamide

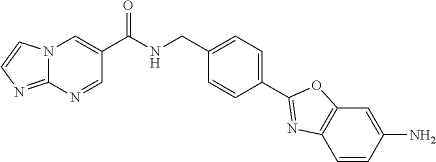 [SMILES: Nc1ccc2nc(-c3ccc(CNC(=O)c4cnc5nccn5c4)cc3)oc2c1]

The title compounds were obtained through the processes of Steps A, B, C, and D below.

### Step A: Preparation of [4-(6-bromo-1,3-benzoxazol-2-yl)phenyl]methanamine

The title compound (5.4 g, 89.8%) was obtained in the same manner as in Step A of Example 1 using 4-(aminomethyl)benzoic acid (3.0 g, 19.85 mmol) and 2-amino-5-bromo-phenol (3.73 g, 19.85 mmol).

¹H NMR (400 MHz, CDCl₃-d) 6 ppm 3.90 (s, 2H), 7.37-7.44 (m, 3H), 7.55 (d, 1H), 7.67 (d, 1H), 8.12 (d, 2H).

### Step B: Preparation of N-[[4-(6-bromo-1,3-benzoxazol-2-yl)phenyl]methyl]imidazo[1,2-a]pyrimidin-6-carboxamide

The title compound (0.5 g, 36.4%) was obtained in the same manner as in Step B of Example 2 using imidazole[1,2-a]pyrimidin-6-carboxylic acid (1.0 g, 6.13 mmol), oxalyl chloride (4.29 mL, 4.29 mmol), dimethyl formamide (23.58 μL, 306.5 μmol), triethylamine (4.27 mL, 30.65 mmol), and the [4-(6-bromo-1,3-benzoxazol-2-yl)phenyl]methanamine (929.16 mg, 3.06 mmol) obtained in Step A.

¹H NMR (400 MHz, DMSO-d₆) δ ppm 9.54 (d, 1H), 9.42 (t, 1H), 9.00 (d, 1H), 8.18 (d, 2H), 8.14 (d, 1H), 8.05 (d, 1H), 7.83 (d, 1H), 7.77 (d, 1H), 7.56-7.65 (m, 3H), 4.65 (d, 2H).

### Step C: Preparation of tert-butyl N-[2-[4-[(imidazo[1,2-a]pyrimidin-6-carbonylamino)methyl]phenyl]-1,3-benzoxazol-6-yl]carbamate

The title compound (10.9 mg, 3.4%) was obtained in the same manner as in Step C of Example 15 using the N-[[4-(6-bromo-1,3-benzoxazol-2-yl)phenyl]methyl]imidazo[1,2-a]pyrimidin-6-carboxamide (300 mg, 669.24 μmol) obtained in Step B, tert-butyl carbamate (235.2 mg, 20.1 mmol), tris(dibenzylideneacetone)dipalladium(0) (61.28 mg, 66.92 μmol), Xantphos (77.45 mg, 133.85 μmol), and cesium carbonate (654.15 mg, 2.01 mmol).

MS [M+H]=485 (M+1)

### Step D: Preparation of N-[[4-(6-amino-1,3-benzoxazol-2-yl)phenyl]methyl]imidazo[1,2-a]pyrimidin-6-carboxamide

The tert-butyl N-[2-[4-[(imidazo[1,2-a]pyrimidin-6-carbonylamino)methyl]phenyl]-1,3-benzoxazol-6-yl]carbamate (36.0 mg, 74.3 μmol) obtained in Step C was dissolved in methanol (1 mL), and a 4N hydrochloric acid 1,4-dioxane solution was added, and then the resulting solution was stirred at room temperature for 12 hours. The reaction solution was concentrated under reduced pressure to obtain a residue, which was purified through prep-HPLC to obtain the title compound (1.7 mg, 5.9%).

¹H NMR (400 MHz, DMSO-d₆) δ ppm 9.59 (s, 1H), 9.38-9.51 (m, 1H), 9.08 (d, 1H), 8.03-8.16 (m, 3H), 7.86-7.98 (m, 1H), 7.56 (d, 2H), 7.36-7.49 (m, 1H), 7.22 (s, 1H), 7.09 (s, 1H), 6.83-7.03 (m, 1H), 6.58-6.76 (m, 1H), 4.63 (d, 2H).

### Example 27: Preparation of N-[[4-[5-(3-pyridyl)-1,3-benzoxazol-2-yl]phenyl]methyl]imidazo[1,2-a]pyrimidin-6-carboxamide

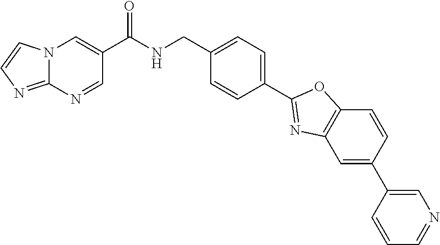 [SMILES: O=C(NCc1ccc(-c2nc3cc(-c4cccnc4)ccc3o2)cc1)c1cnc2nccn2c1]

The title compounds were obtained through the processes of Steps A and B below.

### Step A: Preparation of [4-[5-(3-pyridyl)-1,3-benzoxazol-2-yl]phenyl]methanamine

The [4-(5-bromo-1,3-benzoxazol-2-yl)phenyl]methanamine (200 mg, 659.73 μmol) obtained in Step A of Example 15 was dissolved in ethanol (2 mL) and water (0.2 mL), 3-pyridylboronic acid (121.64 mg, 989.6 μmol), tetrakis(triphenylphosphine)palladium(0) (38.12 mg, 32.99 μmol), and sodium carbonate (279.7 mg, 2.64 mmol) were added, and then the resulting solution was stirred under nitrogen at 80° C. for 16 hours. After lowering the reaction solution to room temperature, water (100 mL) was added and then the resulting solution was extracted twice with ethyl acetate. After washing an organic layer with a sodium chloride aqueous solution, a filtrate dried and filtered with anhydrous sodium sulfate was concentrated under reduced pressure and purified through column chromatography to obtain the title compound (104 mg, 52.3%).

$$MS [ M + H ] = 302 ( M + 1 )$$

### Step B: Preparation of N-[[4-[5-(3-pyridyl)-1,3-benzoxazol-2-yl]phenyl]methyl]imidazo[1,2-a]pyrimidin-6-carboxamide

The title compound (101.7 mg, 66.4%) was obtained in the same manner as in Step B of Example 2 using imidazole[1,2-a]pyrimidin-6-carboxylic acid (112 mg, 686.56 μmol), oxalyl chloride (480.78 μL, 5.49 mmol), triethylamine (477.8 μL, 3.43 mmol), and the [4-[5-(3-pyridyl)-1,3-benzoxazol-2-yl]phenyl]methanamine (103.44 mg, 343.28 μmol) obtained in Step A.

¹H NMR (400 MHz, DMSO-d₆) δ ppm 9.64 (d, 1H), 9.53 (t, 1H), 9.15 (d, 1H), 9.06 (d, 1H), 8.67 (dd, 1H), 8.33-8.37 (m, 1H), 8.21-8.25 (m, 3H), 8.16 (d, 1H), 8.01 (d, 1H), 7.94 (d, 1H), 7.83 (d, 1H), 7.64 (d, 3H), 4.67 (d, 2H).

### Example 28: Preparation of N-[[5-(6-methyl-1,3-benzoxazol-2-yl)-2-pyridyl]methyl]imidazo[1,2-a]pyrimidin-6-carboxamide

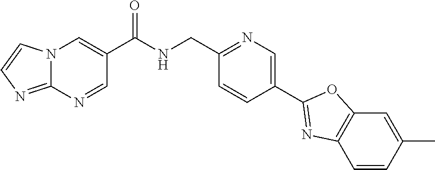 [SMILES: Cc1ccc2nc(-c3ccc(CNC(=O)c4cnc5nccn5c4)nc3)oc2c1]

### Step A: Preparation of [5-(6-methyl-1,3-benzoxazol-2-yl)-2-pyridyl]methanamine

The title compound (0.7 g, 20.2%) was obtained in the same manner as in Step A of Example 1 using the 6-(aminomethyl)pyridin-3-carboxylic acid (2.2 g, 14.46 mmol) obtained in Preparation Example 7 and 2-amino-5-methyl-phenol (1.78 g, 14.46 mmol).

$$MS [ M + H ] = 240 ( M + 1 )$$

### Step B: Preparation of N-[[5-(6-methyl-1,3-benzoxazol-2-yl)-2-pyridyl]methyl]imidazo[1,2-a]pyrimidin-6-carboxamide

The title compound (200 mg, 84.6%) was obtained in the same manner as in Step B of Example 2 using imidazole[1,2-a]pyrimidin-6-carboxylic acid (200 mg, 1.23 mmol), oxalyl chloride (861.37 μL, 9.84 mmol), dimethyl formamide (4.73 μL, 61.5 μmol), triethylamine (856.01 μL, 6.15 mmol), and the [5-(6-methyl-1,3-benzoxazol-2-yl)-2-pyridyl]methanamine (147.15 mg, 615.0 μmol) obtained in Step A.

¹H NMR (400 MHz, DMSO-d₆) δ ppm 9.62 (d, 1H), 9.59 (t, 1H), 9.29 (d, 1H), 9.10 (d, 1H), 8.52 (d, 1H), 8.12 (d1H), 7.94 (d, 1H), 7.73 (t, 1H), 7.62 (d, 2H), 7.25 (d, 1H), 4.75 (d, 2H), 2.53 (s, 3H).

### Example 29: Preparation of ethyl 2-[4-[(imidazo[1,2-a]pyrimidin-6-carbonylamino)methyl]phenyl]-1,3-benzoxazol-5-carboxylate

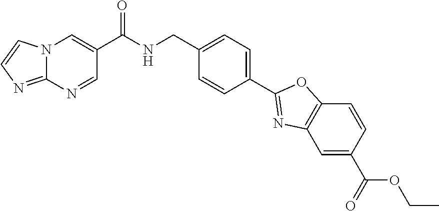 [SMILES: CCOC(=O)c1ccc2oc(-c3ccc(CNC(=O)c4cnc5nccn5c4)cc3)nc2c1]

The title compounds were obtained through the processes of Steps A and B below.

### Step A: Preparation of ethyl 2-[4-(aminomethyl)phenyl]-1,3-benzoxazol-5-carboxylate

The title compound (1.0 g, 56.7%) was obtained in the same manner as in Step A of Example 1 using 4-(aminomethyl)benzoic acid (0.9 g, 5.95 mmol) and ethyl 3-amino-4-hydroxy-benzoate (1.08 g, 5.95 mmol).

$$MS [ M + H ] = 297 ( M + 1 )$$

### Step B: Preparation of ethyl 2-[4-[(imidazo[1,2-a]pyrimidin-6-carbonylamino)methyl]phenyl]-1,3-benzoxazol-5-carboxylate

The title compound (17.8 mg, 3.3%) was obtained in the same manner as in Step B of Example 2 using imidazole[1,2-a]pyrimidin-6-carboxylic acid (200 mg, 1.23 mmol), oxalyl chloride (861.37 μL, 9.84 mmol), dimethyl formamide (4.73 μL, 61.5 μmol), triethylamine (856.01 μL, 6.15 mmol), and the ethyl 2-[4-(aminomethyl)phenyl]-1,3-benzoxazol-5-carboxylate (182.24 mg, 615.0 μmol) obtained in Step A.

¹H NMR (400 MHz, DMSO-d₆) δ ppm 9.55 (d, 1H), 9.40 (t, 1H), 9.00 (d, 1H), 8.30 (d, 1H), 8.25 (d, 2H), 8.10 (d, 2H), 7.95 (d, 1H), 7.85 (d, 1H), 7.65 (d, 2H), 4.70 (d, 2H), 4.3-4.4 (m, 2H), 1.40 (s, 3H).

### Example 30: Preparation of N-[[4-[5-(hydroxymethyl)-1,3-benzoxazol-2-yl]phenyl]methyl]imidazo[1,2-a]pyrimidin-6-carboxamide

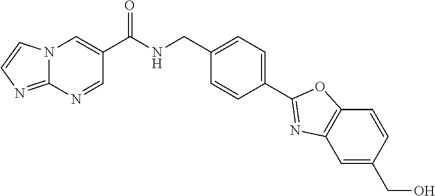 [SMILES: O=C(NCc1ccc(-c2nc3cc(CO)ccc3o2)cc1)c1cnc2nccn2c1]

The title compound was obtained through the processes of Steps A, B, and C.

### Step A: Preparation of methyl 2-[4-(aminomethyl)phenyl]-1,3-benzoxazol-5-carboxylate

The title compound (3.2 g, 57.1%) was obtained in the same manner as in Step A of Example 1 using 4-(aminomethyl)benzoic acid (3.0 g, 19.85 mmol) [symbol] methyl 3-amino-4-hydroxy-benzoate (3.32 g, 19.85 mmol).

¹H NMR (400 MHz, CDCl₃) δ ppm 8.45 (d, 1H), 8.23 (d, 2H), 8.11 (dd, 1H), 7.61 (d, 1H), 7.50 (d, 2H), 3.99 (s, 2H), 3.97 (s, 3H), 1.71-2.06 (m, 2H).

### Step B: Preparation of [2-[4-(aminomethyl)phenyl]-1,3-benzoxazol-5-yl]methanol

The methyl 2-[4-(aminomethyl)phenyl]-1,3-benzoxazol-5-carboxylate (1.00 g, 3.54 mmol) obtained in Step A was dissolved in tetrahydrofuran (30 mL), lithium aluminum hydroxide (174.78 mg, 4.61 mmol) was added at 0° C., and then the resulting solution was stirred at room temperature for 12 hours. After lowering the reaction solution to 0° C., water was slowly added and the resulting solution was stirred for 30 minutes. The produced solid was removed and concentrated under reduced pressure to obtain the title compound (0.8 g, 88.8%).

¹H NMR (400 MHz, DMSO-d₆) δ ppm 8.15 (d, 2H), 7.75 (d, 2H), 7.60 (d, 2H), 7.40 (d, 1H), 4.65 (d, 2H), 3.76-3.86 (m, 2H)

### Step C: Preparation of N-[[4-[5-(hydroxymethyl)-1,3-benzoxazol-2-yl]phenyl]methyl]imidazo[1,2-a]pyrimidin-6-carboxamide

The title compound (42.7 mg, 5.8%) was obtained in the same manner as in Step B of Example 2 using imidazole[1,2-a]pyrimidin-6-carboxylic acid (300 mg, 1.84 mmol), oxalyl chloride (1.29 mL, 14.71 mmol), dimethyl formamide (7.07 μL, 91.95 μmol), triethylamine (1.28 mL, 9.19 mmol), and the [2-[4-(aminomethyl)phenyl]-1,3-benzoxazol-5-yl]methanol (233.81 mg, 919.49 μmol) obtained in Step B.

¹H NMR (400 MHz, DMSO-d₆) δ ppm 9.55 (d, 1H), 9.45 (t, 1H), 9.05 (d, 1H), 8.20 (d, 2H), 8.10 (d, 1H), 7.90 (d, 1H), 7.75 (d, 2H), 7.60 (d, 2H), 7.40 (s, 1H), 4.55-4.65 (m, 4H).

### Example 31: Preparation of N-ethyl-2-[4-[(imidazo[1,2-a]pyrimidin-6-carbonxylamino)methyl]phenyl]-1,3-benzoxazol-5-carboxamide

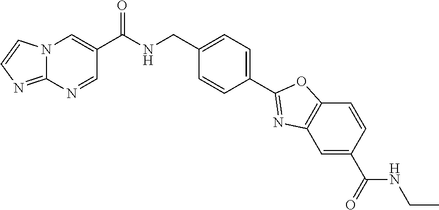 [SMILES: CCNC(=O)c1ccc2oc(-c3ccc(CNC(=O)c4cnc5nccn5c4)cc3)nc2c1]

The title compounds were obtained through the processes of Steps A and B below.

### Step A: Preparation of methyl 2-[4-[(imidazo[1,2-a]pyrimidin-6-carbonylamino)methyl]phenyl]-1,3-benzoxazol-5-carboxylate

The title compound (677 mg, unpurified) was obtained in the same manner as in Step B of Example 2 using imidazole[1,2-a]pyrimidin-6-carboxylic acid (0.5 g, 3.06 mmol), oxalyl chloride (2.15 mL, 24.52 mmol), dimethyl formamide (11.79 μL, 153.25 μmol), triethylamine (2.13 mL, 15.32 mmol), and the methyl 2-[4-(aminomethyl)phenyl]-1,3-benzoxazol-5-carboxylate (432.61 mg, 1.53 mmol) obtained in Step A of Example 31.

[M+H]=428 (M+1)

### Step B: Preparation of 2-[4-[(imidazo[1,2-a]pyrimidin-6-carbonylamino)methyl]phenyl]-1,3-benzoxazol-5-carboxylic acid

The title compound (97 mg, unpurified) was obtained in the same manner as in Step C of Example 5 using the methyl 2-[4-[(imidazo[1,2-a]pyrimidin-6-carbonylamino)methyl]phenyl]-1,3-benzoxazol-5-carboxylate (100 mg, 233.97 μmol) obtained in Step A and lithium iodide (93.95 mg, 701.9 μmol).

[M+H]=414 (M+1)

### Step C: Preparation of N-ethyl-2-[4-[(imidazo[1,2-a]pyrimidin-6-carbonylamino)methyl]phenyl]-1,3-benzoxazol-5-carboxamide

The 2-[4-[(imidazo[1,2-a]pyrimidin-6-carbonylamino)methyl]phenyl]-1,3-benzoxazol-5-carboxylic acid (97 mg, 234.65 μmol) obtained in Step B was dissolved in dimethyl formamide (3 mL), 1-propanephosphonic acid anhydrous solution (418.66 μL, 703.94 mol, purity: 50%) and N,N-diisopropylethylamine (204.36 μL, 1.17 mmol) were added, and then the resulting solution was stirred at room temperature for 30 minutes. In addition, ethylamine hydrochloride (191.34 mg, 2.35 mmol) was added, and then the resulting solution was stirred at room temperature for 12 hours. The reaction solution was concentrated under reduced pressure to obtain a residue, which was purified through prep-HPLC to obtain the title compound (29.2 mg, 28.3%).

¹H NMR (400 MHz, DMSO-d₆) δ ppm 9.56 (d, 1H), 9.44 (t, 1H), 9.03 (d, 1H), 8.59 (t, 1H), 8.27 (d, 1H), 8.18-8.25 (m, 2H), 8.07 (d, 1H), 7.83-7.96 (m, 3H), 7.62 (d, 2H), 4.65 (d, 2H), 1.21-1.30 (m, 2H), 1.15 (t, 3H).

### Example 32: Preparation of 2-[4-[(imidazo[1,2-a]pyrimidin-6-carbonylamino)methyl]phenyl]-N,N-dimethyl-1,3-benzoxazol-5-carboxamide

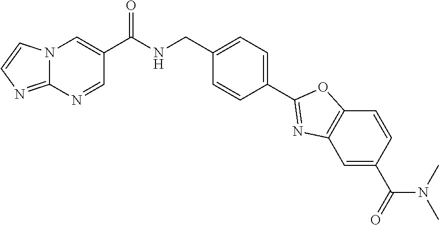 [SMILES: CN(C)C(=O)c1ccc2oc(-c3ccc(CNC(=O)c4cnc5nccn5c4)cc3)nc2c1]

The title compound was obtained through the process of Step A below.

### Step A: Preparation of 2-[4-[(imidazo[1,2-a]pyrimidin-6-carbonylamino)methyl]phenyl]-N,N-dimethyl-1,3-benzoxazol-5-carboxamide

The title compound (8.3 mg, 9.1%) was obtained in the same manner as in Step C of Example 32 using the 2-[4-[(imidazo[1,2-a]pyrimidin-6-carbonylamino)methyl]phenyl]-1,3-benzoxazol-5-carboxylic acid (86 mg, 208.04 μmol) obtained in Step B of Example 32, 1-propanephosphonic acid anhydrous solution (371.18 μL, 624.12 mol, purity: 50%), N,N-diisopropylethylamine (543.54 μL, 3.12 mmol), and N-methylmethanamine hydrochloride (169.64 mg, 2.08 mmol).

¹H NMR (400 MHz, DMSO-d₆) δ ppm 9.58 (d, 1H), 9.48 (t, 1H), 9.07 (d, 1H), 8.20 (d, 2H), 8.10 (d, 1H), 7.92 (d, 1H), 7.82-7.86 (m, 2H), 7.62 (d, 2H), 7.43-7.51 (m, 1H), 4.61-4.70 (m, 2H), 2.93-3.03 (m, 6H).

### Example 33: Preparation of N-[[4-[5-(azetidin-1-carbonyl)-1,3-benzoxazol-2-yl]phenyl]methyl]imidazo[1,2-a]pyrimidin-6-carboxamide

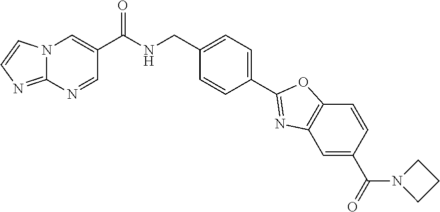 [SMILES: O=C(NCc1ccc(-c2nc3cc(C(=O)N4CCC4)ccc3o2)cc1)c1cnc2nccn2c1]

The title compound was obtained through the process of Step A below.

### Step A: Preparation of N-[[4-[5-(azetidin-1-carbonyl)-1,3-benzoxazol-2-yl]phenyl]methyl]imidazo[1,2-a]pyrimidin-6-carboxamide

The title compound (10.7 mg, 16.9%) was obtained in the same manner as in Step C of Example 32 using the 2-[4-[(imidazo[1,2-a]pyrimidin-6-carbonylamino)methyl]phenyl]-1,3-benzoxazol-5-carboxylic acid (58 mg, 140.31 μmol) obtained in Step B of Example 32, 1-propanephosphonic acid anhydrous solution (250.33 μL, 420.92 mol, purity: 50%), N,N-diisopropylethylamine (366.57 μL, 2.1 mmol), and azetidine hydrochloride (131.26 mg, 1.4 mmol).

¹H NMR (400 MHz, DMSO-d₆) δ ppm 9.54 (d, 1H), 9.37-9.45 (m, 1H), 8.96-9.03 (m, 1H), 8.18-8.24 (m, 2H), 7.99-8.07 (m, 2H), 7.81-7.89 (m, 2H), 7.70 (dd, 1H), 7.59-7.66 (m, 2H), 4.65 (d, 2H), 4.36 (t, 2H), 4.08 (t, 2H), 2.22-2.30 (m, 2H).

### Example 34: Preparation of N-[[4-[5-(pyrrolidine-1-carbonyl)-1,3-benzoxazol-2-yl]phenyl]methyl]imidazo[1,2-a]pyrimidin-6-carboxamide

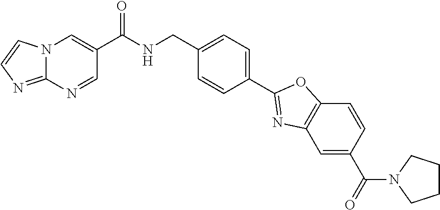 [SMILES: O=C(NCc1ccc(-c2nc3cc(C(=O)N4CCCC4)ccc3o2)cc1)c1cnc2nccn2c1]

The title compound was obtained through the process of Step A below.

### Step A: Preparation of N-[[4-[5-(pyrrolidine-1-carbonyl)-1,3-benzoxazol-2-yl]phenyl]methyl]imidazo[1,2-a]pyrimidin-6-carboxamide

The title compound (29.0 mg, 26.8%) was obtained in the same manner as in Step C of Example 32 using the 2-[4-[(imidazo[1,2-a]pyrimidin-6-carbonylamino)methyl]phenyl]-1,3-benzoxazol-5-carboxylic acid (96 mg, 232.23 μmol) obtained in Step B of Example 32, 1-propanephosphoric acid anhydrous solution (202.25 μL, 696.69 mol, purity: 50%), N,N-diisopropylethylamine (202.25 μL, 1.16 mmol), and pyrrolidine (58.16 μL, 696.69 μmol).

¹H NMR (400 MHz, DMSO-d₆) δ ppm 9.60 (d, 1H), 9.49 (t, 1H), 9.10 (d, 1H), 8.20 (d, 2H), 8.12 (d, 1H), 7.91-7.98 (m, 2H), 7.83 (d, 1H), 7.56-7.67 (m, 3H), 4.65 (d, 2H), 3.13-3.25 (m, 4H), 1.78-1.92 (m, 4H).

### Example 35: Preparation of N-[[2-fluoro-4-(6-methyl-1,3-benzoxazol-2-yl)phenyl]methyl]imidazo[1,2-a]pyrimidin-6-carboxamide

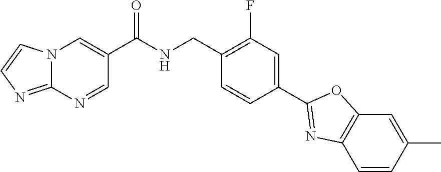 [SMILES: Cc1ccc2nc(-c3ccc(CNC(=O)c4cnc5nccn5c4)c(F)c3)oc2c1]

The title compounds were obtained through the processes of Steps A and B below.

### Step A: Preparation of [2-fluoro-4-(6-methyl-1,3-benzoxazol-2-yl)phenyl]methanamine

The title compound (1.0 g, 66.0%) was obtained in the same manner as in Step A of Example 1 using the 4-(aminomethyl)-3-fluoro-benzoic acid (1.0 g, 5.91 mmol) obtained in Preparation Example 8 and 2-amino-5-methyl-phenol (0.728 g, 5.91 mmol).

¹H NMR (400 MHz, DMSO-d₆) δ ppm 7.97-8.01 (m, 1H), 7.81-7.86 (m, 1H), 7.72-7.77 (m, 1H), 7.66-7.70 (m, 1H), 7.58-7.61 (m, 1H), 7.22-7.27 (m, 1H), 4.00-4.23 (m, 2H), 3.83-3.86 (m, 2H), 3.18 (s, 3H).

### Step B: Preparation of N-[[2-fluoro-4-(6-methyl-1,3-benzoxazol-2-yl)phenyl]methyl]imidazo[1,2-a]pyrimidin-6-carboxamide

The title compound (26.4 mg, 10.7%) was obtained in the same manner as in Step B of Example 2 using imidazole[1,2-a]pyrimidin-6-carboxylic acid (200 mg, 1.23 mmol), oxalyl chloride (858.57 μL, 9.81 mmol), dimethyl formamide (4.72 μL, 61.3 μmol), triethylamine (853.22 μL, 6.13 mmol), and the [2-fluoro-4-(6-methyl-1,3-benzoxazol-2-yl)phenyl]methanamine (224.42 mg, 613.0 mol, purity: 70%) obtained in Step A.

¹H NMR (400 MHz, DMSO-d₆) δ ppm 9.56 (d, 1H), 9.32-9.48 (m, 1H), 9.02 (d, 1H), 8.08 (d, 1H), 8.02 (dd, 1H), 7.96 (dd, 1H), 7.87 (d, 1H), 7.66-7.74 (m, 2H), 7.63 (s, 1H), 7.27 (d, 1H), 4.67 (d, 2H), 2.49 (s, 3H).

### Example 36: Preparation of N-[(4-oxazolo[4,5-c]pyridin-2-ylphenyl)methyl]imidazo[1,2-a]pyrimidin-6-carboxamide

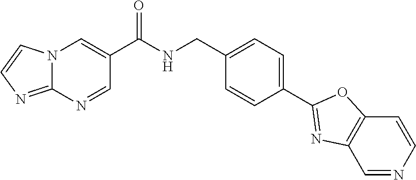 [SMILES: O=C(NCc1ccc(-c2nc3cnccc3o2)cc1)c1cnc2nccn2c1]

The title compounds were obtained through the processes of Steps A and B below.

### Step A: Preparation of (4-oxazolo[4,5-c]pyridin-2-ylphenyl)methanamine

The title compound (0.55 g, 73.8%) was obtained in the same manner as in Step A of Example 1 using 4-(aminomethyl)benzoic acid (0.5 g, 3.31 mmol) and the 3-aminopyridin-4-ol (364.22 mg, 3.31 mmol) obtained in Preparation Example 9.

MS [M+H]=226 (M+1)

### Step B: Preparation of N-[(4-oxazolo[4,5-c]pyridin-2-ylphenyl)methyl]imidazo[1,2-a]pyrimidin-6-carboxamide

The title compound (4.2 mg, 1.2%) was obtained in the same manner as in Step B of Example 2 using imidazole[1,2-a]pyrimidin-6-carboxylic acid (150 mg, 919.49 μmol), oxalyl chloride (643.92 μL, 7.36 mmol), dimethyl formamide (3.54 μL, 45.97 μmol), triethylamine (639.91 μL, 4.6 mmol), and the (4-oxazolo[4,5-c]pyridin-2-ylphenyl)methanamine (147.94 mg, 459.75 mol, purity: 70%) obtained in Step A.

¹H NMR (400 MHz, DMSO-d₆) δ ppm 9.55-9.60 (m, 1H), 9.42-9.49 (m, 1H), 9.11-9.18 (m, 1H), 9.02-9.07 (m, 1H), 8.60-8.64 (m, 1H), 8.21-8.25 (m, 2H), 8.06-8.11 (m, 1H), 7.92-7.97 (m, 1H), 7.86-7.90 (m, 1H), 7.62-7.68 (m, 2H), 4.65-4.69 (m, 2H).

### Example 37: Preparation of N-[[4-(5-ethylsulfonyl-1,3-benzoxazol-2-yl)phenyl]methyl]imidazo[1,2-a]pyrimidin-6-carboxamide

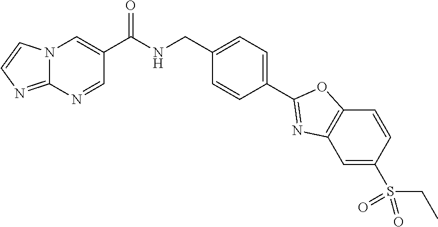 [SMILES: CCS(=O)(=O)c1ccc2oc(-c3ccc(CNC(=O)c4cnc5nccn5c4)cc3)nc2c1]

The title compounds were obtained through the processes of Steps A and B below.

### Step A: Preparation of [4-(5-ethylsulfonyl-1,3-benzoxazol-2-yl)phenyl]methanamine

The title compound (1.3 g, 82.8%) was obtained in the same manner as in Step A of Example 1 using 4-(aminomethyl)benzoic acid (0.75 g, 4.96 mmol) and 2-amino-4-ethylsulfonyl-phenol (998.48 mg, 4.96 mmol).

¹H NMR (400 MHz, DMSO-d₆) δ ppm 1.10-1.16 (m, 3H), 3.17 (q, 2H), 3.88 (br d, 2H), 7.11-7.21 (m, 1H), 7.54 (br d, 1H), 7.64 (d, 1H), 7.89-7.96 (m, 2H), 8.23 (t, 2H).

### Step B: Preparation of N-[[4-(5-ethylsulfonyl-1,3-benzoxazol-2-yl)phenyl]methyl]imidazo[1,2-a]pyrimidin-6-carboxamide

The title compound (12.0 mg, 4.2%) was obtained in the same manner as in Step B of Example 2 using imidazole[1,2-a]pyrimidin-6-carboxylic acid (200 mg, 1.23 mmol), oxalyl chloride (858.54 μL, 9.81 mmol), dimethyl formamide (4.72 μL, 61.3 μmol), triethylamine (853.21 μL, 6.13 mmol), and the [4-(5-ethylsulfonyl-1,3-benzoxazol-2-yl)phenyl]methanamine (193.94 mg, 613.0 μmol) obtained in Step A.

¹H NMR (400 MHz, DMSO-d₆) δ ppm 9.55-9.60 (m, 1H), 9.42-9.49 (m, 1H), 9.02-9.07 (m, 1H), 8.30-8.25 (m, 1H), 8.18-8.23 (m, 2H), 8.06-8.11 (m, 2H), 7.92-7.97 (m, 1H), 7.85-7.70 (m, 1H), 7.62-7.68 (m, 2H), 4.65-4.69 (m, 2H), 2.50-2.45 (m, 2H), 1.10-1.15 (m, 3H).

### Example 38: Preparation of N-[[4-[5-(benzenesulfonyl)-1,3-benzoxazol-2-yl]phenyl]methy]imidazo[1,2-a]pyrimidin-6-carboxamide

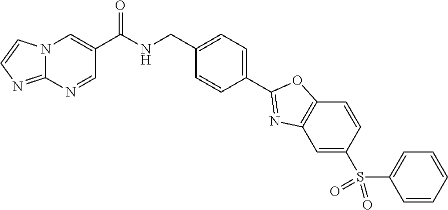 [SMILES: O=C(NCc1ccc(-c2nc3cc(S(=O)(=O)c4ccccc4)ccc3o2)cc1)c1cnc2nccn2c1]

### Step A: Preparation of [4-[5-(benzenesulfonyl)-1,3-benzoxazol-2-yl]phenyl]methanamine

The title compound (2.0 g, 83.0%) was obtained in the same manner as in Step A of Example 1 using 4-(aminomethyl)benzoic acid (1.0 g, 6.62 mmol) and the 2-amino-4-(benzenesulfonyl)phenol (1.65 mg, 6.62 mmol) obtained in Preparation Example 10.

¹H NMR (400 MHz, CDCl₃) δ ppm 3.92 (s, 2H), 7.42-7.48 (m, 4H), 7.48-7.51 (m, 1H), 7.77 (d, 1H), 7.88 (m, 1H), 7.92 (m, 2H), 8.12-8.19 (m, 3H).

### Step B: Preparation of N-[[4-[5-(benzenesulfonyl)-1,3-benzoxazol-2-yl]phenyl]methyl]imidazo[1,2-a]pyrimidin-6-carboxamide

The title compound (29.83 mg, 2.4%) was obtained in the same manner as in Step B of Example 2 using imidazole[1,2-a]pyrimidin-6-carboxylic acid (200 mg, 1.23 mmol), oxalyl chloride (858.54 μL, 9.81 mmol), dimethyl formamide (4.72 μL, 61.3 μmol), triethylamine (853.21 μL, 6.13 mmol), and the [4-[5-(benzenesulfonyl)-1,3-benzoxazol-2-yl]phenyl]methanamine (223.39 mg, 613.0 μmol) obtained in Step C.

¹H NMR (400 MHz, DMSO-d₆) δ ppm 9.51-9.53 (m, 1H), 9.41 (t, 1H), 8.95-9.00 (m, 1H), 8.39-8.45 (m, 1H), 8.16-8.25 (m, 2H), 8.01-8.05 (m, 5H), 7.82 (d, 1H), 7.66-7.71 (m, 1H), 7.60-7.65 (m, 4H), 4.63-4.66 (m, 2H).

### Example 39: Preparation of N-[[4-(5,6-difluoro-1,3-benzoxazol-2-yl)phenyl]methyl]imidazo[1,2-a]pyrimidin-6-carboxamide

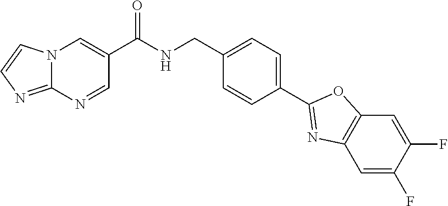 [SMILES: O=C(NCc1ccc(-c2nc3cc(F)c(F)cc3o2)cc1)c1cnc2nccn2c1]

The title compounds were obtained through the processes of Steps A and B below.

### Step A: Preparation of [4-(5,6-difluoro-1,3-benzoxazol-2-yl)phenyl]methanamine

The title compound (480.0 mg, 97.8%) was obtained in the same manner as in Step A of Example 1 using 4-(aminomethyl)benzoic acid (285 mg, 1.89 mmol) and the 2-amino-4,5-difluoro-phenol (273.58 mg, 1.89 mmol) obtained in Preparation Example 11.

¹H NMR (400 MHz, CDCl₃) δ ppm 8.18 (d, 2H), 7.53-7.60 (m, 1H), 7.50 (d, 2H), 7.43 (dd, 1H), 3.99 (s, 2H).

### Step B: Preparation of N-[[4-(5,6-difluoro-1,3-benzoxazol-2-yl)phenyl]methyl]imidazo[1,2-a]pyrimidin-6-carboxamide

The title compound (34.0 mg, 13.7%) was obtained in the same manner as in Step B of Example 2 using imidazole[1,2-a]pyrimidin-6-carboxylic acid (200 mg, 1.23 mmol), oxalyl chloride (858.54 μL, 9.81 mmol), dimethyl formamide (4.72 μL, 61.3 μmol), triethylamine (853.21 μL, 6.13 mmol), and the [4-(5,6-difluoro-1,3-benzoxazol-2-yl)phenyl]methanamine (159.53 mg, 613.0 μmol) obtained in Step A.

¹H NMR (400 MHz, DMSO-d₆) δ ppm 9.57 (d, 1H), 9.46 (s, 1H), 9.04 (d, 1H), 8.13-8.18 (m, 2H), 8.09-8.13 (m, 1H), 8.08 (d, 1H), 7.94-8.03 (m, 1H), 7.88 (d, 1H), 7.61 (d, 2H), 4.65 (d, 2H).

### Example 40: Preparation of N-[[4-(4,6-difluoro-1,3-benzoxazol-2-yl)phenyl]methyl]imidazo[1,2-a]pyrimidin-6-carboxamide

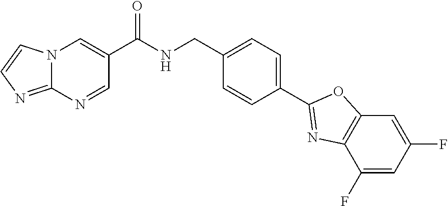 [SMILES: O=C(NCc1ccc(-c2nc3c(F)cc(F)cc3o2)cc1)c1cnc2nccn2c1]

The title compounds were obtained through the processes of Steps A and B below.

### Step A: Preparation of [4-(4,6-difluoro-1,3-benzoxazol-2-yl)phenyl]methanamine

The title compound (390 mg, 22.7%) was obtained in the same manner as in Step A of Example 1 using 4-(aminomethyl)benzoic acid (1.0 g, 6.62 mmol) and the 2-amino-3,5-difluoro-phenol (959.94 mg, 6.62 mmol) obtained in Preparation Example 12.

¹H NMR (400 MHz, CDCl₃) δ ppm 8.16-8.26 (m, 2H), 7.50 (d, 2H), 7.17 (d, 1H), 6.86-6.95 (m, 1H), 3.99 (s, 2H).

### Step B: Preparation of N-[[4-(4,6-difluoro-1,3-benzoxazol-2-yl)phenyl]methyl]imidazo[1,2-a]pyrimidin-6-carboxamide

The title compound (38.1 mg, 15.3%) was obtained in the same manner as in Step B of Example 2 using imidazole[1,2-a]pyrimidin-6-carboxylic acid (200 mg, 1.23 mmol), oxalyl chloride (858.54 μL, 9.81 mmol), dimethyl formamide (4.72 μL, 61.3 μmol), triethylamine (853.21 μL, 6.13 mmol), and the [4-(4,6-difluoro-1,3-benzoxazol-2-yl)phenyl]methanamine (160.05 mg, 613.0 μmol) obtained in Step A.

¹H NMR (400 MHz, DMSO-d₆) δ ppm 9.57 (d, 1H), 9.45 (t, 1H), 9.04 (d, 1H), 8.18 (d, 2H), 8.08 (d, 1H), 7.89 (d, 1H), 7.72-7.76 (m, 1H), 7.62 (d, 2H), 7.38-7.45 (m, 1H), 4.65 (d, 2H).

### Example 41: Preparation of N-[[4-[6-chloro-5-(trifluoromethyl)-1,3-benzoxazol-2-yl]phenyl]methyl]imidazo[1,2-a]pyrimidin-6-carboxamide

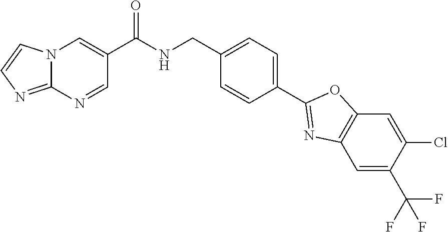 [SMILES: O=C(NCc1ccc(-c2nc3cc(C(F)(F)F)c(Cl)cc3o2)cc1)c1cnc2nccn2c1]

The title compounds were obtained through the processes of Steps A and B below.

### Step A: Preparation of [4-[6-chloro-5-(trifluoromethyl)-1,3-benzoxazol-2-yl]phenyl]methanamine

The title compound (400 mg, 37.0%) was obtained in the same manner as in Step A of Example 1 using 4-(aminomethyl)benzoic acid (500 mg, 3.31 mmol) and the 2-amino-5-chloro-4-(trifluoromethyl)phenol (699.81 mg, 3.31 mmol) obtained in Preparation Example 13.

¹H NMR (400 MHz, CDCl₃) δ ppm 8.22 (d, 2H), 8.10 (s, 1H), 7.75 (s, 1H), 7.52 (d, 2H), 7.27 (s, 1H), 4.01 (s, 2H).

### Step B: Preparation of N-[[4-[6-chloro-5-(trifluoromethyl)-1,3-benzoxazol-2-yl]phenyl]methyl]imidazo[1,2-a]pyrimidin-6-carboxamide

The title compound (59.2 mg, 20.5%) was obtained in the same manner as in Step B of Example 2 using imidazole[1,2-a]pyrimidin-6-carboxylic acid (200 mg, 1.23 mmol), oxalyl chloride (858.54 μL, 9.81 mmol), dimethyl formamide (4.72 μL, 61.3 μmol), triethylamine (853.21 μL, 6.13 mmol), and the [4-[6-chloro-5-(trifluoromethyl)-1,3-benzoxazol-2-yl]phenyl]methanamine (200.27 mg, 613.0 μmol) obtained in Step A.

¹H NMR (400 MHz, DMSO-d₆) δ ppm 9.54 (d, 1H), 9.43 (s, 1H), 8.99 (d, 1H), 8.35 (d, 2H), 8.20 (d, 2H), 8.05 (d, 1H), 7.82 (d, 1H), 7.64 (d, 2H), 4.66 (d, 2H).

### Example 42: Preparation of N-[[4-[5-chloro-6-(trifluoromethyl)-1,3-benzoxazol-2-yl]phenyl]methyl]imidazo[1,2-a]pyrimidin-6-carboxamide

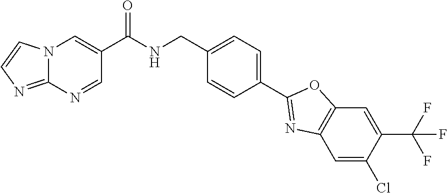 [SMILES: O=C(NCc1ccc(-c2nc3cc(Cl)c(C(F)(F)F)cc3o2)cc1)c1cnc2nccn2c1]

The title compounds were obtained through the processes of Steps A and B below.

### Step A: Preparation of [4-[5-chloro-6-(trifluoromethyl)-1,3-benzoxazol-2-yl]phenyl]methanamine

The title compound (430 mg, 45.2%) was obtained in the same manner as in Step A of Example 1 using 4-(aminomethyl)benzoic acid (440 mg, 2.91 mmol) and the 2-amino-4-chloro-5-(trifluoromethyl)phenol (615.83 mg, 2.91 mmol) obtained in Preparation Example 14.

¹H NMR (400 MHz, CDCl₃) δ ppm 8.24 (d, 2H), 7.83-7.99 (m, 2H), 7.47-7.58 (m, 2H), 4.02 (s, 2H), 2.11 (s, 2H).

### Step B: Preparation of N-[[4-[5-chloro-6-(trifluoromethyl)-1,3-benzoxazol-2-yl]phenyl]methyl]imidazo[1,2-a]pyrimidin-6-carboxamide

The title compound (21.1 mg, 9.7%) was obtained in the same manner as in Step B of Example 2 using imidazole[1,2-a]pyrimidin-6-carboxylic acid (150 mg, 459.75 μmol), oxalyl chloride (643.92 μL, 7.36 mmol), dimethyl formamide (3.54 μL, 45.97 μmol), triethylamine (639.91 μL, 4.60 mmol), and the [4-[5-chloro-6-(trifluoromethyl)-1,3-benzoxazol-2-yl]phenyl]methanamine (150.2 mg, 459.75 μmol) obtained in Step A.

¹H NMR (400 MHz, DMSO-d₆) δ ppm 9.53 (d, 1H), 9.43 (t, 1H), 8.99 (d, 1H), 8.43 (s, 1H), 8.23 (d, 2H), 8.21 (s, 1H), 8.05 (d, 1H), 7.82 (d, 1H), 7.65 (d, 2H), 4.66 (d, 2H).

### Example 43: Preparation of N-[[4-(5-methoxy-6-methyl-1,3-benzoxazol-2-yl)phenyl]methyl]imidazo[1,2-a]pyrimidin-6-carboxamide

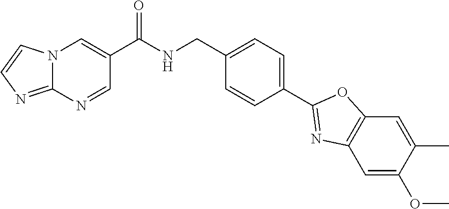 [SMILES: COc1cc2nc(-c3ccc(CNC(=O)c4cnc5nccn5c4)cc3)oc2cc1C]

The title compounds were obtained through the processes of Steps A and B below.

### Step A: Preparation of [4-(5-methoxy-6-methyl-1,3-benzoxazol-2-yl)phenyl]methanamine

The title compound (372.7 mg, 18.8%) was obtained in the same manner as in Step A of Example 1 using 4-(aminomethyl)benzoic acid (300 mg, 1.98 mmol) and the 2-amino-4-methoxy-5-methyl-phenol (304 mg, 1.98 mmol) obtained in Preparation Example 15.

¹H NMR (400 MHz, DMSO-d₆) δ ppm 8.05-8.12 (m, 2H), 7.50-7.61 (m, 3H), 7.31 (s, 1H), 3.86 (s, 3H), 3.80 (s, 2H), 2.28 (s, 2H), 1.68 (s, 3H).

### Step B: Preparation of N-[[4-(5-methoxy-6-methyl-1,3-benzoxazol-2-yl)phenyl]methyl]imidazo[1,2-a]pyrimidin-6-carboxamide

The title compound (28.3 mg, 18.6%) was obtained in the same manner as in Step B of Example 2 using imidazole[1,2-a]pyrimidin-6-carboxylic acid (120 mg, 735.59 μmol), oxalyl chloride (515.12 μL, 5.88 mmol), dimethyl formamide (2.83 μL, 36.78 μmol), triethylamine (511.93 μL, 3.68 mmol), and the [4-(5-methoxy-6-methyl-1,3-benzoxazol-2-yl)phenyl]methanamine (98.68 mg, 367.8 μmol) obtained in Step A.

¹H NMR (400 MHz, DMSO-d₆) δ ppm 9.53 (d, 1H), 9.37-9.42 (m, 1H), 8.99 (d, 1H), 8.13 (d, 2H), 8.04 (d, 1H), 7.82 (d, 1H), 7.57-7.60 (m, 3H), 7.33 (s, 1H), 4.63 (d, 2H), 3.86 (s, 3H), 2.28 (s, 3H).

### Example 44: Preparation of N-[[4-(5-ethyl-1,3-benzoxazol-2-yl)phenyl]methyl]imidazo[1,2-a]pyrimidin-6-carboxamide

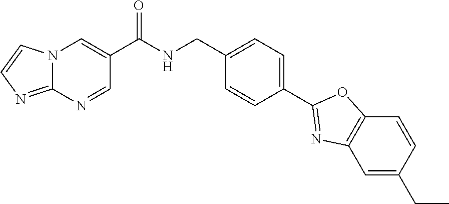 [SMILES: CCc1ccc2oc(-c3ccc(CNC(=O)c4cnc5nccn5c4)cc3)nc2c1]

The title compounds were obtained through the processes of Steps A and B below.

### Step A: Preparation of [4-(5-ethyl-1,3-benzoxazol-2-yl)phenyl]methanamine

The title compound (0.62 g, 69.7%) was obtained in the same manner as in Step A of Example 1 using 4-(aminomethyl)benzoic acid (533 mg, 3.53 mmol) and the 2-amino-4-ethyl-phenol (483.69 mg, 3.53 mmol) obtained in Preparation Example 16.

¹H NMR (400 MHz, DMSO-d₆) δ ppm 8.12 (d, 2H), 7.67 (d, 1H), 7.61 (s, 1H), 7.57 (d, 2H), 7.27 (dd, 1H), 3.81 (s, 2H), 3.17 (d, 1H), 2.74 (m, 2H), 1.94 (s, 1H), 1.24 (t, 3H).

### Step B: Preparation of N-[[4-(5-ethyl-1,3-benzoxazol-2-yl)phenyl]methyl]imidazo[1,2-a]pyrimidin-6-carboxamide

The title compound (46.4 mg, 25.4%) was obtained in the same manner as in Step B of Example 2 using imidazole[1,2-a]pyrimidin-6-carboxylic acid (150 mg, 919.49 μmol), oxalyl chloride (643.9 μL, 7.36 mmol), dimethyl formamide (3.54 μL, 45.97 μmol), triethylamine (639.91 μL, 4.6 mmol), and the [4-(5-ethyl-1,3-benzoxazol-2-yl)phenyl]methanamine (116.00 mg, 459.75 μmol) obtained in Step A.

¹H NMR (400 MHz, DMSO-d₆) δ ppm 9.58 (d, 1H), 9.42-9.49 (m, 1H), 9.06 (d, 1H), 8.18 (d, 2H), 8.10 (d, 1H), 7.91 (s, 1H), 7.68 (d, 1H), 7.57-7.64 (m, 3H), 7.28 (dd, 1H), 4.65 (d, 2H), 2.74 (m, 2H), 1.24 (t, 3H).

### Example 45: Preparation of N-[[4-(6-ethyl-1,3-benzoxazol-2-yl)phenyl]methyl]imidazo[1,2-a]pyrimidin-6-carboxamide

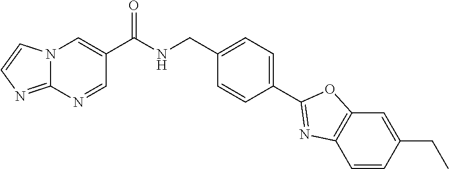 [SMILES: CCc1ccc2nc(-c3ccc(CNC(=O)c4cnc5nccn5c4)cc3)oc2c1]

The title compounds were obtained through the processes of Steps A and B below.

### Step A: Preparation of [4-(6-ethyl-1,3-benzoxazol-2-yl)phenyl]methanamine

The title compound (0.467 g, 35.8%) was obtained in the same manner as in Step A of Example 1 using 4-(aminomethyl)benzoic acid (780 mg, 5.16 mmol) and the 2-amino-5-ethyl-phenol (707.84 mg, 5.16 mmol) obtained in Preparation Example 17.

$$MS [ M + H ] = 253 ( M + 1 )$$

### Step B: Preparation of N-[[4-(6-ethyl-1,3-benzoxazol-2-yl)phenyl]methyl]imidazo[1,2-a]pyrimidin-6-carboxamide

The title compound (54.3 mg, 29.7%) was obtained in the same manner as in Step B of Example 2 using imidazole[1,2-a]pyrimidin-6-carboxylic acid (150 mg, 919.49 μmol), oxalyl chloride (643.90 μL, 7.36 mmol), dimethyl formamide (3.54 μL, 45.97 μmol), triethylamine (639.91 μL, 4.60 mmol), and the [4-(6-ethyl-1,3-benzoxazol-2-yl)phenyl]methanamine (116.0 mg, 459.75 μmol) obtained in Step A.

¹H NMR (400 MHz, DMSO-d₆) δ ppm 9.57 (d, 1H), 9.44 (t, 1H), 9.05 (d, 1H), 8.16 (d, 2H), 8.09 (s, 1H), 7.89 (s, 1H), 7.69 (d, 1H), 7.58-7.65 (m, 3H), 7.27 (d, 1H), 4.64 (d, 2H), 2.76 (m, 2H), 1.25 (t, 3H).

### Example 46: Preparation of N-[[4-(5-amino-1,3-benzoxazol-2-yl)phenyl]methyl]imidazo[1,2-a]pyrimidin-6-carboxamide

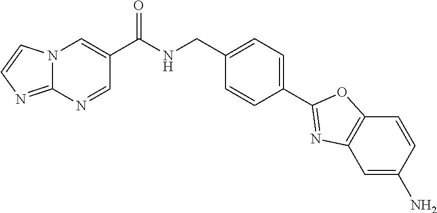 [SMILES: Nc1ccc2oc(-c3ccc(CNC(=O)c4cnc5nccn5c4)cc3)nc2c1]

The title compounds were obtained through the processes of Steps A and B below.

### Step A: Preparation of tert-butyl N-[2-[4-[(imidazo[1,2-a]pyrimidin-6-carbonylamino)methyl]phenyl]-1,3-benzoxazol-5-yl]carbamate

The title compound (129 mg, 23.87%) was obtained in the same manner as in Step C of Example 15 using the N-[[4-(5-bromo-1,3-benzoxazol-2-yl)phenyl]methyl]imidazo[1,2-a]pyrimidin-6-carboxamide (500 mg, 1.12 mmol) obtained in Step B of Example 15, tert-butyl carbamate (391.99 mg, 3.35 mmol), tris(dibenzylideneacetone)dipalladium(0) (153.21 mg, 167.31 μmol), Xantphos (159.52 mg, 334.62 μmol), and potassium triphosphate (462.46 mg, 3.35 mmol).

$$MS [ M + H ] = 485 ( M + 1 )$$

### Step B: Preparation of N-[[4-(5-amino-1,3-benzoxazol-2-yl)phenyl]methyl]imidazo[1,2-a]pyrimidin-6-carboxamide

The title compound (12.8 mg, 12.5%) was obtained in the same manner as in Step D of Example 27 using the tert-butyl N-[2-[4-[(imidazo[1,2-a]pyrimidin-6-carbonylamino)methyl]phenyl]-1,3-benzoxazol-5- [symbol] ]carbamate (129 mg, 266.25 μmol) obtained in Step A and 4N hydrochloric acid methanol solution (4 mL).

¹H NMR (400 MHz, DMSO-d₆) δ ppm 9.54 (d, 1H), 9.41 (t, 1H), 8.99 (d, 1H), 8.11 (d, 2H), 8.05 (d, 1H), 7.82 (d, 1H), 7.56 (d, 2H), 7.41 (d, 1H), 6.86 (d, 1H), 6.66 (dd, 1H), 4.93-5.34 (m, 2H), 4.62 (d, 2H).

### Example 47: Preparation of N-[[4-[5-(methanesulfonamino)-1,3-benzoxazol-2-yl]phenyl]methyl]imidazo[1,2-a]pyrimidin-6-carboxamide

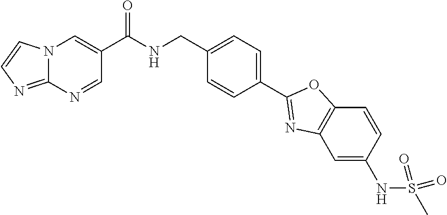 [SMILES: CS(=O)(=O)Nc1ccc2oc(-c3ccc(CNC(=O)c4cnc5nccn5c4)cc3)nc2c1]

The title compounds were obtained through the processes of Steps A, B, C, and D below.

### Step A: Preparation of tert-butyl N-[[4-(5-bromo-1,3-benzoxazol-2-yl)phenyl]methyl]carbamate

The [4-(5-bromo-1,3-benzoxazol-2-yl)phenyl]methanamine (1.0 g, 3.3 mmol) obtained in Step A of Example 15 was dissolved in dichloromethane (15 mL), di-tert-butyl dicarbonate (863.91 mg, 3.96 mmol) and triethylamine (500.68 mg, 4.95 mmol) were added, and then the resulting solution was stirred at room temperature for 4 hours. The reaction solution as concentrated under reduced pressure to obtain a residue, which was purified through column chromatography to obtain the title compound (1.0 g, 75.2%).

¹H NMR (400 MHz, CDCl₃) δ ppm 8.21 (d, 2H), 7.91 (s, 1H), 7.43-7.48 (m, 5H), 4.42 (d, 2H), 1.49 (s, 9H).

### Step B: Preparation of tert-butyl N-[[4-[5-(methanesulfonamido)-1,3-benzoxazol-2-yl]phenyl]methyl]carbamate

The title compound (215 mg, 23.1%) was obtained in the same manner as in Step C of Example 15 using the tert-butyl N-[[4-(5-bromo-1,3-benzoxazol-2-yl)phenyl]methyl]carbamate (900 mg, 2.23 mmol) obtained in Step A, cesium carbonate (2.18 g, 6.70 mmol), Xantphos (0.40 mg, 669.53 mmol), and tris(dibenzylideneacetone)dipalladium(0) (204.37 mg, 223.18 μmol).

$$MS [ M + H ] = 418 ( M + 1 )$$

### Step C: Preparation of N-[2-[4-(aminomethyl)phenyl]-1,3-benzoxazol-5-yl]methanesulfonamide

An unpurified result (236 mg) was obtained in the same manner as in Step D of Example 27 using the tert-butyl N-[[4-[5-(methanesulfonamido)-1,3-benzoxazol-2-yl]phenyl]methyl]carbamate (200 mg, 479.07 μmol) obtained in Step B and 4N hydrochloric acid methanol solution (4.0 mL), and used in a subsequent step without purification.

MS [M+H]=318 (M+1)

### Step D: Preparation of N-[[4-[5-(methanesulfonamido)-1,3-benzoxazol-2-yl]phenyl]methyl]imidazo[1,2-a]pyrimidin-6-carboxamide

The title compound (5.6 mg, 3.9%) was obtained in the same manner as in Step B of Example 2 using imidazole[1,2-a]pyrimidin-6-carboxylic acid (100.61 mg, 616.73 μmol), oxalyl chloride (431.89 μL, 4.93 mmol), dimethyl formamide (23.73 μL, 308.37 μmol), triethylamine (429.21 μL, 3.08 mmol), and the N-[2-[4-(aminomethyl)phenyl]-1,3-benzoxazol-5-yl]methanesulfonamide (97.86 mg, 308.37 μmol) obtained in Step C.

¹H NMR (400 MHz, DMSO-d₆) δ ppm 9.79 (s, 1H), 9.53 (d, 1H), 9.40 (s, 1H), 8.99 (d, 1H), 8.18 (d, 2H), 8.05 (d, 1H), 7.82 (d, 1H), 7.77 (d, 1H), 7.61-7.63 (m, 2H), 7.60 (s, 1H), 7.27 (dd, 1H), 4.64 (d, 2H), 2.99 (s, 3H).

### Example 48: Preparation of N-[[4-(5-methyl-1,3-benzothiazol-2-yl)phenyl]methyl]imidazo[1,2-a]pyrimidin-6-carboxamide

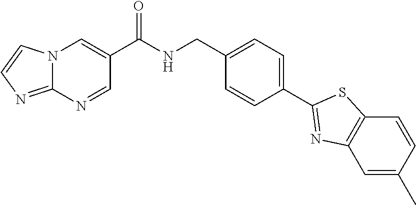 [SMILES: Cc1ccc2sc(-c3ccc(CNC(=O)c4cnc5nccn5c4)cc3)nc2c1]

The title compounds were obtained through the processes of Steps A and B below.

### Step A: Preparation of [4-(5-methyl-1,3-benzothiazol-2-yl)phenyl]methanamine

the 2-bromo-5-methyl-1,3-benzothiazole (251.82 mg, 1.1 mmol) obtained in Step A of Preparation Example 18, and [4-(aminomethyl)phenyl]boronic acid (200 mg, 3.31 mmol) were dissolved in dimethylether (2.5 mL) and water (0.5 mL), 1,1′ bis(diphenylphosphino)ferrocene]dichloropalladium(II) (80.78 mg, 110.1 μmol) and sodium carbonate (351.03 mg, 3.31 mmol) were added, and then the resulting solution was stirred under nitrogen at 80° C. for 12 hours. The reaction solution was concentrated under reduced pressure to obtain a residue, which was purified through column chromatography to obtain the title compound (110 mg, 39.2%).

MS [M+H]=255 (M+1)

### Step B: Preparation of N-[[4-(5-methyl-1,3-benzothiazol-2-yl)phenyl]methyl]imidazo[1,2-a]pyrimidin-6-carboxamide

The title compound (12.4 mg, 7.2%) was obtained in the same manner as in Step B of Example 2 using imidazole[1,2-a]pyrimidin-6-carboxylic acid (140 mg, 858.19 μmol), oxalyl chloride (600.98 μL, 6.87 mmol), dimethyl formamide (3.3 μL, 42.91 μmol), triethylamine (597.25 μL, 4.29 mmol), and the [4-(5-methyl-1,3-benzothiazol-2-yl)phenyl]methanamine (109.14 mg, 429.1 μmol) obtained in Step A.

¹H NMR (400 MHz, DMSO-d₆) δ ppm 9.54 (d, 1H), 9.41 (t, 1H), 9.00 (d, 1H), 8.07-8.08 (m, 1H), 8.05-8.06 (m, 2H), 8.01 (d, 1H), 7.84-7.88 (m, 2H), 7.56 (d, 2H), 7.30 (d, 1H), 4.62 (d, 2H), 2.47 (s, 3H).

### Example 49: Preparation of N-[[4-(5-chloro-1,3-benzothiazol-2-yl)phenyl]methyl]imidazo[1,2-a]pyrimidin-6-carboxamide

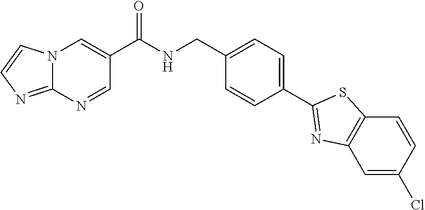 [SMILES: O=C(NCc1ccc(-c2nc3cc(Cl)ccc3s2)cc1)c1cnc2nccn2c1]

The title compounds were obtained through the processes of Steps A and B below.

### Step A: Preparation of [4-(5-chloro-1,3-benzothiazol-2-yl)phenyl]methanamine

The title compound (88 mg, 39.8%) was obtained in the same manner as in Step A of Example 49 using [4-(aminomethyl)phenyl]boronic acid (121.49 mg, 804.74 μmol), 2-bromo-5-chloro-1,3-benzothiazole (200 mg, 804.74 μmol), sodium carbonate (170.59 mg, 1.61 mmol), and [1,1′ bis(diphenyl[phosphino)ferrocene]dichloropalladium(II) (58.88 mg, 80.47 μmol).

$$MS [ M + H ] = 275 ( M + 1 )$$

### Step B: Preparation of N-[[4-(5-chloro-1,3-benzothiazol-2-yl)phenyl]methyl]imidazo[1,2-a]pyrimidin-6-carboxamide

The title compound (17.2 mg, 12.9%) was obtained in the same manner as in Step B of Example 2 using imidazole[1,2-a]pyrimidin-6-carboxylic acid (103.32 mg, 633.33 μmol), oxalyl chloride (443.51 μL, 5.07 mmol), dimethyl formamide (2.44 μL, 31.67 μmol), triethylamine (440.76 μL, 3.17 mmol), and the [4-(5-chloro-1,3-benzothiazol-2-yl)phenyl]methanamine (88 mg, 316.67 μmol) obtained in Step A.

¹H NMR (400 MHz, DMSO-d₆) δ ppm 9.53 (d, 1H), 9.41 (t, 1H), 8.99 (d, 1H), 8.20 (d, 1H), 8.15 (d, 1H), 8.09 (d, 2H), 8.05 (d, 1H), 7.83 (d, 1H), 7.58 (d, 2H), 7.52 (dd, 1H), 4.63 (d, 2H).

### Example 50: Preparation of N-[[4-(6-chloro-1,3-benzothiazol-2-yl)phenyl]methyl]imidazo[1,2-a]pyrimidin-6-carboxamide

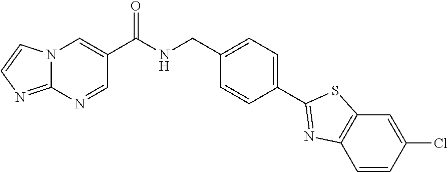 [SMILES: O=C(NCc1ccc(-c2nc3ccc(Cl)cc3s2)cc1)c1cnc2nccn2c1]

### Step A: Preparation of [4-(6-chloro-1,3-benzothiazol-2-yl)phenyl]methanamine

The title compound (100 mg, 27.6%) was obtained in the same manner as in Step A of Example 49 using [4-(aminomethyl)phenyl]boronic acid (199.28 mg, 804.74 μmol), 2-bromo-6-chloro-1,3-benzothiazole (328 mg, 804.74 μmol), sodium carbonate (170.59 mg, 1.61 mmol), and [1,1′ bis(diphenylphosphino)ferrocene]dichloropalladium (II) (58.88 mg, 80.47 μmol).

$$MS [ M + H ] = 275 ( M + 1 )$$

### Step B: Preparation of N-[[4-(6-chloro-1,3-benzothiazol-2-yl)phenyl]methyl]imidazo[1,2-a]pyrimidin-6-carboxamide

The title compound (12.1 mg, 7.9%) was obtained in the same manner as in Step B of Example 2 using imidazole[1,2-a]pyrimidin-6-carboxylic acid (118.74 mg, 727.89 μmol), oxalyl chloride (509.73 μL, 5.82 mmol), dimethyl formamide (2.80 μL, 36.39 μmol), triethylamine (506.56 μL, 3.64 mmol), and the [4-(6-chloro-1,3-benzothiazol-2-yl)phenyl]methanamine (100 mg, 363.94 μmol) obtained in Step A.

¹H NMR (400 MHz, DMSO-d₆) δ ppm 9.59 (d, 1H), 9.47 (t, 1H), 9.08 (d, 1H), 8.33 (d, 1H), 8.10 (d, 2H), 8.04-8.08 (m, 2H), 7.93 (s, 1H), 7.56-7.60 (m, 3H), 4.63 (d, 2H).

### Example 51: Preparation of N-[[4-(5-methoxy-1,3-benzothiazol-2-yl)phenyl]methyl]imidazo[1,2-a]pyrimidin-6-carboxamide

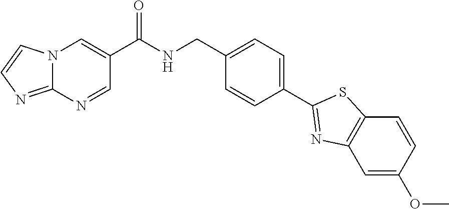 [SMILES: COc1ccc2sc(-c3ccc(CNC(=O)c4cnc5nccn5c4)cc3)nc2c1]

The title compounds were obtained through the processes of Steps A and B below.

### Step A: Preparation of [4-(5-methoxy-1,3-benzothiazol-2-yl)phenyl]methanamine

The title compound (196.4 mg, 69.3%) was obtained in the same manner as in Step A of Example 49 using the 2-bromo-5-methoxy-1,3-benzothiazole (256.01 mg, 1.05 mmol), [4-(aminomethyl)phenyl]boronic acid (190 mg, 1.26 mmol) obtained in Preparation Example 19, sodium carbonate (333.47 mg, 3.15 mmol), and [1,1′ bis(diphenylphosphino)ferrocene]dichloropalladium(II) (76.74 mg, 104.88 μmol).

$$MS [ M + H ] = 275 ( M + 1 )$$

### Step B: Preparation of N-[[4-(5-methoxy-1,3-benzothiazol-2-yl)phenyl]methyl]imidazo[1,2-a]pyrimidin-6-carboxamide

The title compound (32 mg, 10.6%) was obtained in the same manner as in Step B of Example 2 using imidazole[1,2-a]pyrimidin-6-carboxylic acid (237.02 mg, 1.45 mmol), oxalyl chloride (1.02 mL, 11.62 mmol), triethylamine (1.01 mL, 7.26 mmol), and the [4-(5-methoxy-1,3-benzothiazol-2-yl)phenyl]methanamine (196.4 mg, 726.47 μmol) obtained in Step A.

¹H NMR (400 MHz, DMSO-d₆) δ ppm 9.51-9.55 (m, 1H), 9.36-9.41 (m, 1H), 8.99 (d, 1H), 8.03-8.08 (m, 3H), 8.00 (d, 1H), 7.82 (d, 1H), 7.59-7.62 (m, 1H), 7.53-7.59 (m, 2H), 7.08-7.12 (m, 1H), 4.60-4.64 (m, 2H), 3.85-3.89 (m, 3H).

### Example 52: Preparation of N-[[4-(6-methoxy-1,3-benzothiazol-2-yl)phenyl]methyl]imidazo[1,2-a]pyrimidin-6-carboxamide

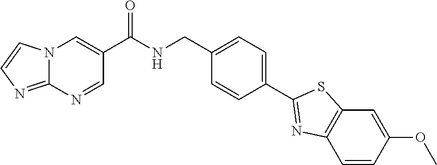 [SMILES: COc1ccc2nc(-c3ccc(CNC(=O)c4cnc5nccn5c4)cc3)sc2c1]

The title compounds were obtained through the processes of Steps A and B below.

### Step A: Preparation of [4-(6-methoxy-1,3-benzothiazol-2-yl)phenyl]methanamine

The title compound (50 mg, 33.5%) was obtained in the same manner as in Step A of Example 49 using [4-(aminomethyl)phenyl]boronic acid (100 mg, 662.38 μmol), 2-bromo-6-methoxy-1,3-benzothiazole (134.74 mg, 551.98 μmol), sodium carbonate (175.51 mg, 1.66 mmol), and [1,1′ bis(diphenylphosphino)ferrocene]dichloropalladium(II) (40.39 mg, 55.2 mol).

$$MS [ M + H ] = 271 ( M + 1 )$$

### Step B: Preparation of N-[[4-(6-methoxy-1,3-benzothiazol-2-yl)phenyl]methyl]imidazo[1,2-a]pyrimidin-6-carboxamide

The title compound (9.5 mg, 12.4%) was obtained in the same manner as in Step B of Example 2 using imidazole[1,2-a]pyrimidin-6-carboxylic acid (60.34 mg, 369.89 μmol), oxalyl chloride (259.04 μL, 2.96 mmol), dimethyl formamide (1.42 μL, 18.49 μmol), triethylamine (257.42 μL, 1.85 mmol), and the [4-(6-methoxy-1,3-benzothiazol-2-yl)phenyl]methanamine (50 mg, 184.95 μmol) obtained in Step A.

¹H NMR (400 MHz, DMSO-d₆) δ ppm 9.53 (d, 1H), 9.39 (t, 1H), 8.98 (d, 1H), 8.03-8.05 (m, 2H), 8.01 (s, 1H), 7.94 (d, 1H), 7.82 (s, 1H), 7.72 (d, 1H), 7.55 (d, 2H), 7.12-7.16 (m, 1H), 4.61 (d, 2H), 3.85 (s, 3H)

### Example 53: Preparation of N-[[4-(5-phenoxy-1,3-benzoxazol-2-yl)phenyl]methyl]imidazo[1,2-a]pyrimidin-6-carboxamide

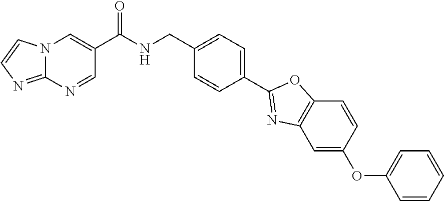 [SMILES: O=C(NCc1ccc(-c2nc3cc(Oc4ccccc4)ccc3o2)cc1)c1cnc2nccn2c1]

The title compounds were obtained through the processes of Steps A and B below.

### Step A: Preparation of [4-(5-phenoxy-1,3-benzoxazol-2-yl)phenyl]methanamine

The title compound (54 mg, 16.7%) was obtained in the same manner as in Step A of Example 1 using 4-(aminomethyl)benzoic acid (206 mg, 1.02 mmol) and the 2-amino-4-phenoxy-phenol (154.75 mg, 1.02 mmol) obtained in Preparation Example 20.

$$MS [ M + H ] = 317 ( M + 1 )$$

### Step B: Preparation of N-[[4-(5-phenoxy-1,3-benzoxazol-2-yl)phenyl]methyl]imidazo[1,2-a]pyrimidin-6-carboxamide

The title compound (24.3 mg, 52.7%) was obtained in the same manner as in Step B of Example 2 using imidazole[1,2-a]pyrimidin-6-carboxylic acid (109 mg, 668.17 μmol), oxalyl chloride (467.90 μL, 5.35 mmol), dimethyl formamide (2.57 μL, 33.41 μmol), triethylamine (465.0 μL, 3.34 mmol), and the [4-(5-phenoxy-1,3-benzoxazol-2-yl)phenyl]methanamine (105.69 mg, 334.08 μmol) obtained in Step A.

¹H NMR (400 MHz, DMSO-d₆) δ ppm 9.54 (d, 1H), 9.40 (s, 1H), 8.99 (d, 1H), 8.18 (d, 2H), 8.04 (d, 1H), 7.79-7.84 (m, 2H), 7.61 (d, 2H), 7.45 (d, 1H), 7.39 (t, 2H), 7.10-7.17 (m, 2H), 7.02 (d, 2H), 4.64 (d, 2H).

### Example 54: Preparation of N-[[4-(5-propoxy-1,3-benzoxazol-2-yl)phenyl]methyl]imidazo[1,2-a]pyrimidin-6-carboxamide

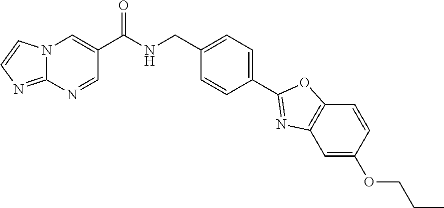 [SMILES: CCCOc1ccc2oc(-c3ccc(CNC(=O)c4cnc5nccn5c4)cc3)nc2c1]

The title compounds were obtained through the processes of Steps A, B, C, and D below.

### Step A: Preparation of tert-butyl N-[[4-[(2-hydroxy-5-propoxy-phenyl)carbamoyl]phenyl]methyl]carbamate

4-[(Tert-butoxycarbonylamino)methyl]benzoic acid (435.82 mg, 1.73 mmol) was dissolved in dimethyl formamide (4 mL), N,N-diisopropylethylamine (317.21 μL, 1.82 mmol) and hexafluorophosphate azabentriazole tetramethyluronium (692.45 mg, 1.82 mmol), and the 2-amino-4-propoxy-phenol (290 mg, 1.73 mmol) obtained in Preparation Example 21 were added, and then the resulting solution was stirred at room temperature for 4 hours. After adjusting the pH of the reaction solution to 4 to 5 using 2N hydrochloric acid aqueous solution, water (100 mL) was added and then extracted twice with ethyl acetate. After washing an organic layer with a sodium chloride aqueous solution, a filtrate dried and filtered with anhydrous sodium sulfate was concentrated under reduced pressure and purified through column chromatography to obtain an unpurified result (0.884 g), and used in a subsequent step without purification.

$$MS [ M + H ] = 401 ( M + 1 )$$

### Step B: Preparation of 4-(aminomethyl)-N-(2-hydroxy-5-propoxy-phenyl)benzamide

An unpurified result (0.7591 g) was obtained in the same manner as in Step D of Example 27 using the tert-butyl N-[[4-[(2-hydroxy-5-propoxy-phenyl)carbamoyl]phenyl]methyl]carbamate (780 mg, 1.95 mmol) obtained in Step A and 4N hydrochloric acid 1,4-dioxane solution (7 mL), and used in a subsequent step without purification.

$$MS [ M + H ] = 301 ( M + 1 )$$

### Step C: Preparation of [4-(5-propoxy-1,3-benzoxazol-2-yl)phenyl]methanamine

The 4-(aminomethyl)-N-(2-hydroxy-5-propoxy-phenyl)benzamide (0.3 g, 998.83 μmol) obtained in Step B was dissolved in toluene (3 mL), 4-methylbenzenesulfonic acid (516 mg, 3.0 mmol) was added, and the resulting solution was stirred under nitrogen at 110° C. for 6 hours. The reaction solution was concentrated under reduced pressure, sodium bicarbonate solution (50 mL) was added, and the resulting solution was extracted twice with dichloromethane. After washing an organic layer with a sodium chloride aqueous solution, a filtrate dried and filtered with anhydrous sodium sulfate was concentrated under reduced pressure and purified through column chromatography to obtain the title compound (87 mg, 30.9%).

$$MS [ M + H ] = 283 ( M + 1 )$$

### Step D: Preparation of N-[[4-(5-propoxy-1,3-benzoxazol-2-yl)phenyl]methyl]imidazo[1,2-a]pyrimidin-6-carboxamide

The title compound (15.3 mg, 11.6%) was obtained in the same manner as in Step B of Example 2 using imidazole[1,2-a]pyrimidin-6-carboxylic acid (100.54 mg, 616.29 μmol), oxalyl chloride (431.57 μL, 4.93 mmol), triethylamine (428.9 μL, 3.08 mmol), and the [4-(5-propoxy-1,3-benzoxazol-2-yl)phenyl]methanamine (87 mg, 308.14 μmol) obtained in Step C.

¹H NMR (400 MHz, DMSO-d₆) δ ppm 9.54-9.56 (m, 1H), 9.41 (t, 1H), 9.01 (d, 1H), 8.14-8.17 (m, 2H), 8.05-8.07 (m, 1H), 7.85 (d, 1H), 7.67 (d, 1H), 7.60 (d, 2H), 7.33 (d, 1H), 6.98-7.02 (m, 1H), 4.64 (d, 2H), 3.97-4.01 (m, 2H), 1.73-1.79 (m, 2H), 1.00 (t, 3H).

### Example 55: Preparation of N-[[4-(5-benzyloxy-1,3-benzoxazol-2-yl)phenyl]methyl]imidazo[1,2-a]pyrimidin-6-carboxamide

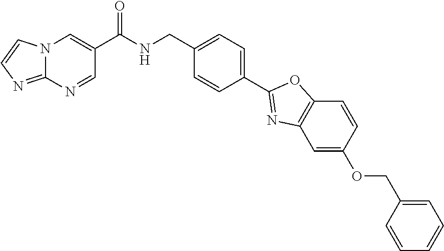 [SMILES: O=C(NCc1ccc(-c2nc3cc(OCc4ccccc4)ccc3o2)cc1)c1cnc2nccn2c1]

The title compound was obtained through the processes of Steps A, B, and C.

### Step A: Preparation of tert-butyl N-[[4-[(5-benzyloxy-2-hydroxy-phenyl)carbamoyl]phenyl]methyl]carbamate

The title compound (1.5 g, unpurified) was obtained in the same manner as in Step A of Example 55 using the 2-amino-4-benzyloxy-phenol (645 mg, 3.0 mmol) obtained in Preparation Example 22, 4-[(tert-butoxycarbonylamino)methyl]benzoic acid (752.97 mg, 3.0 mmol), N,N-diisopropylethylamine (548.04 μL, 3.15 mmol), and hexafluorophosphate azabentriazole tetramethyluronium (1.2 g, 3.15 mmol).

¹H NMR (400 MHz, DMSO-d₆) δ ppm 9.42 (s, 1H), 9.32-9.35 (m, 1H), 7.87-7.93 (m, 2H), 7.36-7.54 (m, 8H), 6.83 (d, 1H), 6.72 (d, 1H), 5.02 (s, 2H), 4.20 (d, 2H), 1.40 (s, 9H).

### Step B: Preparation of [4-(5-benzyloxy-1,3-benzoxazol-2-yl)phenyl]methanamine

The tert-butyl N-[[4-[(5-benzyloxy-2-hydroxy-phenyl)carbamoyl]phenyl]methyl]carbamate (1.08 g, 2.41 mmol) obtained in Step A was dissolved in xylene (15 mL), pyridinium para-toluenesulfonic acid (605.12 mg, 2.41 mmol) was added, and the resulting solution was stirred under nitrogen at 100° C. for 8 hours. The reaction solution was concentrated under reduced pressure to obtain a residue, which was purified through prep-HPLC to obtain the title compound (130 mg, 16.3%).

¹H NMR (400 MHz, DMSO-d₆) δ ppm 8.22 (d, 2H), 7.70 (dd, 3H), 7.48 (d, 2H), 7.38-7.46 (m, 3H), 7.31-7.37 (m, 1H), 7.10 (s, 1H), 5.20 (s, 2H), 4.15 (s, 2H).

### Step C: Preparation of N-[[4-(5-benzyloxy-1,3-benzoxazol-2-yl)phenyl]methyl]imidazo[1,2-a]pyrimidin-6-carboxamide

The title compound (3.5 mg, 4.0%) was obtained in the same manner as in Step B of Example 2 using imidazole[1,2-a]pyrimidin-6-carboxylic acid (60 mg, 367.80 μmol), oxalyl chloride (257.56 μL, 2.94 mmol), triethylamine (255.96 μL, 1.84 mmol), and the [4-(5-benzyloxy-1,3-benzoxazol-2-yl)phenyl]methanamine (60.76 mg, 183.9 μmol) obtained in Step B.

1H NMR (400 MHz, DMSO-d₆) δ ppm 9.53 (d, 1H), 9.40 (t, 1H), 8.99 (d, 1H), 8.15 (d, 2H), 8.04 (s, 1H), 7.82 (s, 1H), 7.69 (d, 1H), 7.59 (d, 2H), 7.47-7.51 (m, 2H), 7.37-7.44 (m, 3H), 7.34 (d, 1H), 7.08 (dd, 1H), 5.18 (s, 2H), 4.63 (d, 2H).

### Example 56: Preparation of N-[[4-(5-phenyl-1,3-benzoxazol-2-yl)phenyl]methyl]imidazo[1,2-a]pyrimidin-6-carboxamide

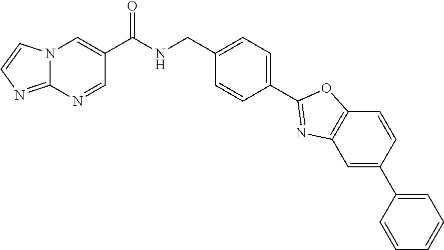 [SMILES: O=C(NCc1ccc(-c2nc3cc(-c4ccccc4)ccc3o2)cc1)c1cnc2nccn2c1]

The title compound was obtained through the processes of Steps A, B, and C.

### Step A: Preparation of [4-(5-phenyl-1,3-benzoxazol-2-yl)phenyl]methanamine

The title compound (74.1 mg, 15.2%) was obtained in the same manner as in Step A of Example 1 using 4-(aminomethyl)benzoic acid (300 mg, 1.62 mmol) and 2-amino-4-phenyl-phenol (244.83 mg, 1.62 mmol).

$$MS [ M + H ] = 301 ( M + 1 )$$

### Step B: Preparation of N-[[4-(5-phenyl-1,3-benzoxazol-2-yl)phenyl]methyl]imidazo[1,2-a]pyrimidin-6-carboxamide

The title compound (15 mg, 13.7%) was obtained in the same manner as in Step B of Example 2 using imidazole[1,2-a]pyrimidin-6-carboxylic acid (80.38 mg, 492.75 μmol), oxalyl chloride (345.08 μL, 3.94 mmol), dimethyl formamide (1.90 μL, 24.64 μmol), triethylamine (342.93 μL, 2.46 mmol), and the [4-(5-phenyl-1,3-benzoxazol-2-yl)phenyl]methanamine (74 mg, 246.38 μmol) obtained in Step A.

¹H NMR (400 MHz, DMSO-d₆) δ ppm 9.64 (d, 1H), 9.52 (t, 1H), 9.15 (d, 1H), 8.23 (s, 1H), 8.21 (s, 1H), 8.16 (d, 1H), 8.06 (d, 1H), 8.01 (s, 1H), 7.87 (d, 1H), 7.75 (s, 1H), 7.71-7.74 (m, 2H), 7.64 (s, 1H), 7.62 (s, 1H), 7.50 (t, 2H), 7.37-7.42 (m, 1H), 4.67 (d, 2H).

### Example 57: Preparation of N-[[4-(6-phenoxy-1,3-benzoxazol-2-yl)phenyl]methyl]imidazo[1,2-a]pyrimidin-6-carboxamide

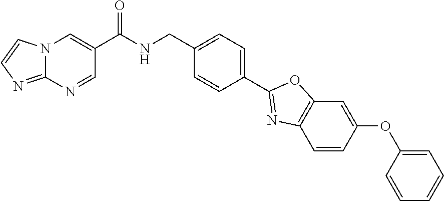 [SMILES: O=C(NCc1ccc(-c2nc3ccc(Oc4ccccc4)cc3o2)cc1)c1cnc2nccn2c1]

The title compounds were obtained through the processes of Steps A, B, C, and D below.

### Step A: Preparation of tert-butyl N-[[4-[(2-hydroxy-5-phenoxy-phenyl)carbamoyl]phenyl]methyl]carbamate

4-[(Tert-butoxycarbonylamino)methyl]benzoic acid (491.5 mg, 1.99 mmol) was dissolved in acetonitrile (4 mL), 2-chloro-1-methylpyridinium iodo (533.26 mg, 2.08 mmol) was added, and then the resulting solution was stirred at room temperature for 3.5 hours. Triethylamine (553.37 μL, 7.96 mmol) was added and stirred at room temperature for 4 hours, the 2-amino-5-phenoxy-phenol (400 mg, 1.99 mmol) obtained in Preparation Example 23 was added, and then the resulting solution was stirred at room temperature for 12 hours. The reaction solution as concentrated under reduced pressure to obtain a residue, which was purified through column chromatography to obtain the title compound (0.92 g, unpurified).

$$MS [ M + H ] = 435 ( M + 1 )$$

### Step B: Preparation of 4-(aminomethyl)-N-(2-hydroxy-5-phenoxy-phenyl)benzamide

An unpurified result (630 mg) was obtained in the same manner as in Step D of Example 27 using the tert-butyl N-[[4-[(2-hydroxy-5-phenoxy-phenyl)carbamoyl]phenyl]methyl]carbamate (820 mg, 1.89 mmol) obtained in Step A and 4N hydrochloric acid 1,4-dioxane solution (8 mL), and used in a subsequent step without purification.

$$MS [ M + H ] = 335 ( M + 1 )$$

### Step C: Preparation of [4-(5-phenoxy-1,3-benzoxazol-2-yl)phenyl]methanamine

The title compound (0.337 g, 56.6%) was obtained in the same manner as in Step C of Example 55 using the 4-(aminomethyl)-N-(2-hydroxy-5-phenoxy-phenyl)benzamide (630 mg, 1.88 mmol) obtained in Step B and 4-methylbenzenesulfonic acid (973.36 mg, 5.65 mmol).

¹H NMR (400 MHz, DMSO-d₆) δ ppm 8.21 (d, 2H), 7.80-7.84 (m, 1H), 7.65-7.70 (m, 2H), 7.48 (s, 2H), 7.40-7.45 (m, 2H), 7.10-7.11 (m, 1H), 7.04-7.09 (m, 2H), 4.13 (s, 2H).

### Step D: Preparation of N-[[4-(6-phenoxy-1,3-benzoxazol-2-yl)phenyl]methyl]imidazo[1,2-a]pyrimidin-6-carboxamide

The title compound (29.6 mg, 22.6%) was obtained in the same manner as in Step B of Example 2 using imidazole[1,2-a]pyrimidin-6-carboxylic acid (92.82 mg, 568.99 μmol), oxalyl chloride (398.45 μL, 4.55 mmol), triethylamine (395.98 μL, 2.84 mmol), and the [4-(5-phenoxy-1,3-benzoxazol-2-yl)phenyl]methanamine (90 mg, 284.49 μmol) obtained in Step C.

¹H NMR (400 MHz, DMSO-d₆) δ ppm 9.52-9.54 (m, 1H), 9.41 (t, 1H), 8.98-9.00 (m, 1H), 8.15 (d, 2H), 8.05 (d, 1H), 7.79-7.83 (m, 2H), 7.60 (d, 2H), 7.47-7.50 (m, 1H), 7.41 (t, 2H), 7.14-7.19 (m, 1H), 7.05-7.12 (m, 3H), 4.64 (d, 2H).

### Example 58: Preparation of N-[[4-[6-(3-pyridyloxy)-1,3-benzoxazol-2-yl]phenyl]methyl]imidazo[1,2-a]pyrimidin-6-carboxamide

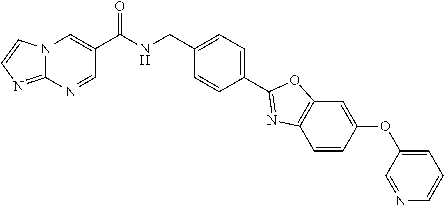 [SMILES: O=C(NCc1ccc(-c2nc3ccc(Oc4cccnc4)cc3o2)cc1)c1cnc2nccn2c1]

The title compounds were obtained through the processes of Steps A and B below.

### Step A: Preparation of [4-[6-(3-pyridyloxy)-1,3-benzoxazol-2-yl]phenyl]methanamine

The title compound (240 mg, 76.5%) was obtained in the same manner as in Step A of Example 1 using 4-(aminomethyl)benzoic acid (200 mg, 989.08 μmol) and the 2-amino-5-(3-pyridyloxy)phenol (149.51 mg, 989.08 μmol) obtained in Step C.

$$MS [ M + H ] = 318 ( M + 1 )$$

### Step B: Preparation of N-[[4-[6-(3-pyridyloxy)-1,3-benzoxazol-2-yl]phenyl]methyl]imidazo[1,2-a]pyrimidin-6-carboxamide

The title compound (6.8 mg, 4.0%) was obtained in the same manner as in Step B of Example 2 using imidazole[1,2-a]pyrimidin-6-carboxylic acid (120 mg, 735.59 μmol), oxalyl chloride (515.12 μL, 5.88 mmol), triethylamine (511.93 μL, 3.68 mmol), and the [4-[6-(3-pyridyloxy)-1,3-benzoxazol-2-yl]phenyl]methanamine (116.72 mg, 367.80 μmol) obtained in Step A.

¹H NMR (400 MHz, DMSO-d₆) δ ppm 9.53 (d, 1H), 9.40 (s, 1H), 8.99 (d, 1H), 8.44 (d, 1H), 8.37-8.41 (m, 1H), 8.16 (d, 2H), 8.04 (d, 1H), 7.81-7.86 (m, 2H), 7.58-7.63 (m, 3H), 7.47 (dd, 1H), 7.44 (d, 1H), 7.17 (dd, 1H), 4.64 (d, 2H).

### Example 59: Preparation of N-[[4-(6-phenyl-1,3-benzoxazol-2-yl)phenyl]methyl]imidazo[1,2-a]pyrimidin-6-carboxamide

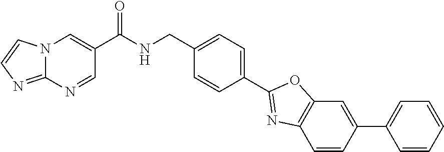 [SMILES: O=C(NCc1ccc(-c2nc3ccc(-c4ccccc4)cc3o2)cc1)c1cnc2nccn2c1]

The title compounds were obtained through the processes of Steps A and B below.

### Step A: Preparation of [4-(6-phenyl-1,3-benzoxazol-2-yl)phenyl]methanamine

The title compound (151 mg, 76.2%) was obtained in the same manner as in Step A of Example 28 using the N-[[4-(6-amino-1,3-benzoxazol-2-yl)phenyl]methyl]imidazo[1,2-a]pyrimidin-6-carboxamide (200 mg, 659.73 μmol) obtained in Step A of Example 27 and phenyl boronic acid (120.66 mg, 989.6 μmol), tetrakis(triphenylphosphine)palladium(0) (38.12 mg, 32.99 μmol), and sodium carbonate (279.7 mg, 2.64 mmol).

$$MS [ M + H ] = 301 ( M + 1 )$$

### Step B: Preparation of N-[[4-(6-phenyl-1,3-benzoxazol-2-yl)phenyl]methyl]imidazo[1,2-a]pyrimidin-6-carboxamide

The title compound (4.0 mg, 3.9%) was obtained in the same manner as in Step B of Example 2 using imidazole[1,2-a]pyrimidin-6-carboxylic acid (75 mg, 459.75 μmol), oxalyl chloride (321.95 μL, 3.68 mmol), triethylamine (319.95 μL, 2.3 mmol), and the [4-(6-phenyl-1,3-benzoxazol-2-yl)phenyl]methanamine (69.04 mg, 229.87 μmol) obtained in Step A.

¹H NMR (400 MHz, DMSO-d₆) δ ppm 9.54 (d, 1H), 9.42 (s, 1H), 8.99 (d, 1H), 8.21 (d, 2H), 8.10 (s, 1H), 8.05 (d, 1H), 7.87 (d, 1H), 7.82 (d, 1H), 7.77 (d, 2H), 7.74 (d, 1H), 7.63 (d, 2H), 7.50 (t, 2H), 7.41 (d, 1H), 4.65 (d, 2H).

### Example 60: Preparation of N-[[4-(5-chlorooxazolo[5,4-b]pyridin-2-yl)phenyl]methyl]imidazo[1,2-a]pyrimidin-6-carboxamide

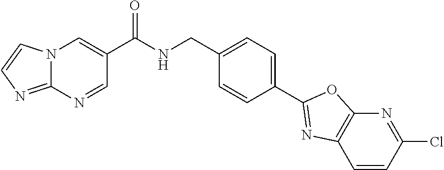 [SMILES: O=C(NCc1ccc(-c2nc3ccc(Cl)nc3o2)cc1)c1cnc2nccn2c1]

### Step A: Preparation of [4-(5-chlorooxazolo[5,4-b]pyridin-2-yl)phenyl]methanamine

The title compound (115 mg, 26.7%) was obtained in the same manner as in Step A of Example 1 using 4-(aminomethyl)benzoic acid (250.96 mg, 1.66 mmol) and the 3-amino-6-chloro-pyridin-2-ol (240 mg, 1.66 mmol) obtained in Preparation Example 25.

$$MS [ M + H ] = 260 ( M + 1 )$$

### Step B: Preparation of N-[[4-(5-chlorooxazolo[5,4-b]pyridin-2-yl)phenyl]methyl]imidazo[1,2-a]pyrimidin-6-carboxamide

The title compound (6.2 mg, 3.5%) was obtained in the same manner as in Step B of Example 2 using imidazole[1,2-a]pyrimidin-6-carboxylic acid (143.75 mg, 881.16 μmol), oxalyl chloride (617.06 μL, 7.05 mmol), triethylamine (613.23 μL, 4.41 mmol), and the [4-(5-chlorooxazolo[5,4-b]pyridin-2-yl)phenyl]methanamine (114.41 mg, 440.58 μmol) obtained in Step A.

¹H NMR (400 MHz, DMSO-d₆) δ ppm 9.54 (d, 1H), 9.40-9.48 (m, 1H), 9.00 (d, 1H), 8.33 (d, 1H), 8.20 (d, 2H), 8.06 (d, 1H), 7.84 (s, 1H), 7.64-7.67 (m, 1H), 7.60-7.64 (m, 2H), 4.65 (d, 2H).

### Example 61: Preparation of N-[[4-(6-methyl-1,3-benzoxazol-2-yl)phenyl]methyl]imidazo[1,2-a]pyridin-6-carboxamide

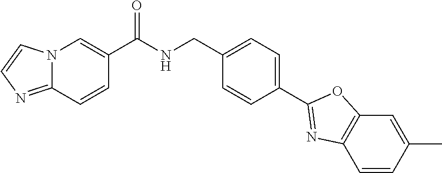 [SMILES: Cc1ccc2nc(-c3ccc(CNC(=O)c4ccc5nccn5c4)cc3)oc2c1]

The title compound was obtained through the process of Step A below.

### Step A: Preparation of N-[[4-(6-methyl-1,3-benzoxazol-2-yl)phenyl]methyl]imidazo[1,2-a]pyridin-6-carboxamide

The title compound (57.79 mg, 24.5%) was obtained in the same manner as in Step B of Example 1 using the [4-(6-methyl-1,3-benzoxazol-2-yl)phenyl]methanamine (146.96 mg, 616.73 μmol), imidazo[1,2-a]pyrimidin-6-carboxylic acid (0.1 g, 616.73 μmol) obtained in Step A of Example 1, and 1-ethyl-3-(3-dimethylaminopropyl)carbodiimide (141.87 mg, 740.08 mol).

1H NMR (400 MHz, DMSO-d₆) δ ppm 9.26 (t, 1H), 9.20 (s, 1H), 8.16 (d, 2H), 8.09 (s, 1H), 7.70-7.75 (m, 1H), 7.62-7.69 (m, 3H), 7.54-7.61 (m, 3H), 7.22 (d, 1H), 4.62 (d, 2H), 2.46 (s, 3H).

### Example 62: Preparation of N-[[4-(6-chlorooxazolo[4,5-c]pyridin-2-yl)phenyl]methyl]imidazo[1,2-a]pyridin-6-carboxamide

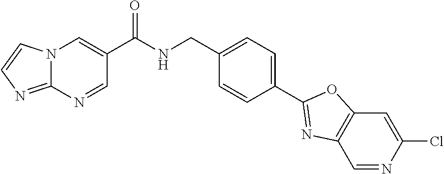 [SMILES: O=C(NCc1ccc(-c2nc3cnc(Cl)cc3o2)cc1)c1cnc2nccn2c1]

### Step A: Preparation of [4-(6-chlorooxazolo[4,5-c]pyridin-2-yl)phenyl]methanamine

The title compound (45 mg, 8.32%) was obtained in the same manner as in Step A of Example 1 using the 5-amino-2-chloro-pyridin-4-ol (301.24 mg, 2.08 mmol) obtained in Preparation Example 26 and 4-(aminomethyl)benzoic acid (315 mg, 2.08 mmol).

$$MS [ M + H ] = 260 ( M + 1 )$$

### Step B: Preparation of N-[[4-(6-chlorooxazolo[4,5-c]pyridin-2-yl)phenyl]methyl]imidazo[1,2-a]pyridin-6-carboxamide

The title compound (28.3 mg, 40.4%) was obtained in the same manner as in Step B of Example 2 using imidazole[1,2-a]pyrimidin-6-carboxylic acid (44.97 mg, 881.16 μmol), oxalyl chloride (242.54 μL, 2.77 mmol), triethylamine (241.03 μL, 1.73 mmol), and the [4-(6-chlorooxazolo[4,5-c]pyridin-2-yl)phenyl]methanamine (56.5 mg, 346.34 μmol) obtained in Step A.

¹H NMR (400 MHz, DMSO-d₆) δ ppm 9.55 (d, 1H), 9.44 (t, 1H), 9.01 (d, 1H), 8.93 (s, 1H), 8.20 (d, 2H), 8.13 (s, 1H), 8.06 (d, 1H), 7.85 (d, 1H), 7.64 (d, 2H), 4.66 (d, 2H).

### Example 63: Preparation of N-[[5-(6-chloro-1,3-benzoxazol-2-yl)-2-pyridyl]methyl]imidazo[1,2-a]pyrimidin-6-carboxamide

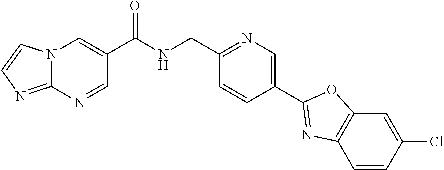 [SMILES: O=C(NCc1ccc(-c2nc3ccc(Cl)cc3o2)cn1)c1cnc2nccn2c1]

### Step A: Preparation of [5-(6-chloro-1,3-benzoxazol-2-yl)-2-pyridyl]methanamine

The title compound (120 mg, 21.9%) was obtained in the same manner as in Step A of Example 1 using the 6-(aminomethyl)pyridin-3-carboxylic acid (301.96 mg, 2.10 mmol) obtained in Preparation Example 7 and 2-amino-5-chloro-phenol (320 mg, 2.10 mmol).

$$MS [ M + H ] = 260 ( M + 1 )$$

### Step B: Preparation of N-[[5-(6-chloro-1,3-benzoxazol-2-yl)-2-pyridyl]methyl]imidazo[1,2-a]pyrimidin-6-carboxamide

The title compound (35.9 mg, 19.3%) was obtained in the same manner as in Step B of Example 2 using imidazole[1,2-a]pyrimidin-6-carboxylic acid (150 mg, 919.49 μmol), oxalyl chloride (643.9 μL, 7.36 mmol), triethylamine (639.91 μL, 4.60 mmol), and the [5-(6-chloro-1,3-benzoxazol-2-yl)-2-pyridyl]methanamine (119.39 mg, 459.75 μmol) obtained in Step A.

1H NMR (400 MHz, DMSO-d₆) δ ppm 9.51 (d, 1H), 9.24-9.33 (m, 2H), 9.00 (d, 1H), 8.50 (d, 1H), 8.02 (s, 1H), 7.97 (s, 1H), 7.84 (d, 1H), 7.81 (s, 1H), 7.68 (d, 1H), 7.49 (d, 1H), 4.75 (d, 2H).

### Example 64: Preparation of N-[1-[4-(6-methyl-1,3-benzooxazol-2-yl)phenyl]ethyl]imidazo[1,2-a]pyrimidin-6-carboxamide

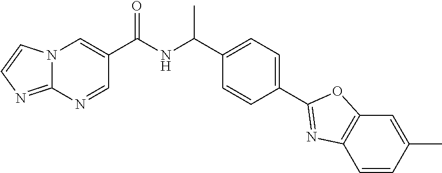 [SMILES: Cc1ccc2nc(-c3ccc(C(C)NC(=O)c4cnc5nccn5c4)cc3)oc2c1]

### Step A: Preparation of 1-[4-(6-methyl-1,3-benzooxzaol-2-yl)phenyl]ethanamine

The title compound (0.25 mg, 54.6%) was obtained in the same manner as in Step A of Example 1 using 4-(1-aminoethyl)benzoic acid (0.3 g, 1.82 mmol) and 2-amino-5-methyl-phenol (223.7 mg, 1.82 mmol).

1H NMR (400 MHz, CDCl₃) δ ppm 8.10-8.16 (m, 2H), 7.56 (d, J=8.12 Hz, 1H), 7.43 (d, J=8.24 Hz, 2H), 7.31 (s, 1H), 7.10 (d, J=8.00 Hz, 1H), 4.14 (q, J=6.56 Hz, 1H), 2.42-2.46 (m, 3H), 1.33-1.38 (m, 3H).

### Step B: Preparation of N-[1-[4-(6-methyl-1,3-benzooxazaol-2-yl)phenyl]ethyl]imidazo[1,2-a]pyrimidin-6-carboxamide

The title compound (149.6 mg, 30.6%) was obtained in the same manner as in Step B of Example 2 using imidazole[1,2-a]pyrimidin-6-carboxylic acid (200 mg, 1.23 mmol), oxalyl chloride (858.57 μL, 9.84 mmol), dimethyl formamide (4.73 μL, 61.30 μmol), triethylamine (856.01 μL, 6.15 mmol), and the 1-[4-(6-methyl-1,3-benzooxazol-2-yl)phenyl]ethanamine (155.17 mg, 615.00 μmol) obtained in Step A.

1H NMR (400 MHz, DMSO-d₆) δ ppm 9.53 (d, J=2.60 Hz, 1H), 9.25 (d, J=1.20 Hz, 1H), 8.99 (d, J=2.60 Hz, 1H), 8.17 (d, J=8.00 Hz, 2H), 8.05 (d, J=1.60 Hz, 1H), 7.83 (d, J=1.60 Hz, 1H), 7.60-7.65 (m, 3H), 7.60 (d, J=1.40 Hz, 1H), 7.25 (d, J=6.00 Hz, 1H), 5.25 (d, J=8.00 Hz, 1H), 2.50 (s, 3H), 1.55 (s, 3H).

### Experimental Example 1: Measurement of NamPT Inhibitory Ability IC₅₀

To test the effect of the compound prepared in each Example on the substrate decomposition rate (Vmax/min) mediated by NamPT enzyme, IC₅₀ was calculated. In this experiment, the NamPT colorimetric assay kit (ab221819) produced by Abcam was used, and Graph prism software was used for IC₅₀ calculation. PRPP, ATP, NMNAT1, NamPT recombinant proteins, and various concentrations of the compound of each Example (half-log step dilution from 100 μM) were added to nicotinamide to synthesize nicotinamide adenine dinucleotide (NAD) from the nicotinamide, and a total of 60 μL of the reaction solution was reacted at room temperature for 1 hour. WST-1 formazan converted by NAD generated by adding WST-1 was measured. The result of the reaction was obtained by the following formula by measuring absorbance using EnVision2105 (PerkinElmer) at 450 nm for 60 minutes at 2-minute intervals.

$$Activity [ {OD}_{450 n m} ] = ( A 2 - A 1 ) / ( T 2 - T 1 ) \qquad [ Mathematical Formula 1 ]$$

In Mathematical Formula 1, T1 and T2 are arbitrary times, and A1 and A2 represent the absorbance measured at T1 and T2 at 450 nm, respectively.

The results of measuring the NamPT inhibition activity of the compound of each Example obtained through the above experiment are shown in Table 1 in the unit of IC₅₀ (nM), and the result values are expressed as follows.

A is IC₅₀<0.05 μM, B is IC₅₀ 0.051 to 0.1 μM, C is IC₅₀ 0.11 to 1 μM, and D is IC₅₀ >1.0 μM.

TABLE 1

| Compound | NamPT IC₅₀ (nM) |
|---|---|
| Example 1 | B |
| Example 2 | B |
| Example 3 | A |
| Example 4 | A |
| Example 5 | C |
| Example 6 | A |
| Example 7 | A |
| Example 8 | A |
| Example 9 | A |
| Example 10 | A |
| Example 11 | A |
| Example 12 | B |
| Example 13 | A |
| Example 14 | A |
| Example 15 | C |
| Example 16 | A |
| Example 17 | B |
| Example 18 | C |
| Example 19 | A |
| Example 20 | C |
| Example 21 | C |
| Example 22 | A |
| Example 23 | A |
| Example 24 | A |
| Example 25 | A |
| Example 26 | B |
| Example 27 | B |
| Example 28 | C |
| Example 29 | A |
| Example 30 | A |
| Example 31 | A |
| Example 32 | A |
| Example 33 | A |
| Example 34 | A |
| Example 35 | A |
| Example 36 | C |
| Example 37 | C |
| Example 38 | A |
| Example 39 | A |
| Example 40 | A |
| Example 41 | A |
| Example 42 | A |
| Example 43 | A |
| Example 44 | A |
| Example 45 | A |
| Example 46 | B |
| Example 47 | A |
| Example 48 | A |
| Example 49 | A |
| Example 50 | B |
| Example 51 | A |
| Example 52 | A |
| Example 53 | A |
| Example 54 | B |
| Example 55 | D |
| Example 56 | C |
| Example 57 | D |
| Example 58 | A |
| Example 59 | D |
| Example 60 | A |
| Example 61 | C |
| Example 62 | B |
| Example 63 | A |
| Example 64 | D |

As seen from Table 1, it was confirmed that the compounds of Examples according to the present invention have excellent NamPT inhibition ability.

### Experimental Example 2: Proliferation Inhibitory Effect (EC₅₀) on NAPRT Negative Cancer Cell Line

A compound showing a low IC₅₀ value was selected from the compounds of the above Examples and treated an NAPRT positive or negative lung cancer cell line with various concentrations, and then the change in cell viability for each cell line was measured. The results are presented in a graph (a few materials known as NamPT inhibitors were used as controls). A total of 9 cancer cells lines (three NAPRT negative cell lines: H322, H661, and H1155; and six NAPRT positive cell lines: H1993, H2122, H2030, H1975, H1299, and HBEC30) were used. 5% fetal bovine serum (Gibco, US) and 1% penicillin/streptomycin (Gibco) were added to a medium, and the cells were cultured in a 37° C. constant temperature incubator containing 5% CO₂. For compound treatment, each type of cells were seeded at 3×10³ cells/well in a 96-well plate (Corning) and then incubated in a 37° C. constant temperature incubator containing 5% CO₂ for 18 hours. Drug treatment was performed by half-log step dilution from 100 μM to 0.1 nM such that the final concentration of DMSO did not exceed 0.5%. Afterwards, the cells were cultured in an incubator containing 5% CO₂ at 37° C. for 72 hours. To confirm the effect of inhibiting the proliferation of cancer cells, a CellTiter-Glo Luminescent Cell Viability Assay (Promega) was used. After treating the cells with 10 μL of CellTiter-Glo luciferase reagent and leaving them for 15 minutes at room temperature, the luminescence of each well was measured using EnVision2105 (PerkinElmer). The change in cell viability by the compound diluted at the concentration of each step was converted into a relative % value for the amount of luminescence shown by DMSO treatment. EC₅₀ is expressed as the concentration of each compound that induces 50% of the maximum luminescence level.

The results of measuring the proliferation inhibition ability of the compound of each Example on nine types of lung cancer cell lines, obtained through the above experiment, are shown in Table 2 in the unit of EC50 (μM). The resulting values are expressed as follows.

A is EC₅₀ <0.05 μM, B is EC₅₀ 0.051 to 0.1 μM, C is EC₅₀ 10 to 100 μM, and D is EC₅₀ >101 μM.

TABLE 2

| | EC₅₀ | EC₅₀ |
|---|---|---|
| Compound | (uM)negative | (uM)positive |
| Example 2 | A | D |
| Example 3 | A | D |
| Example 4 | A | D |
| Example 6 | A | D |
| Example 9 | A | D |
| Example 10 | A | D |
| Example 12 | A | D |
| Example 13 | A | D |
| Example 17 | B | D |
| Example 19 | B | D |
| Example 22 | B | D |
| Example 31 | A | D |
| Example 32 | A | C |
| Example 33 | A | D |
| Example 34 | A | C |
| Example 38 | B | D |

As seen from Table 2, it can be confirmed that the compounds of Examples according to the present invention selectively inhibited proliferation in NAPRT negative lung cancer cell lines, and seen that the proliferation inhibitory effect was also excellent.

### Experimental Example 3: Verification of Pharmacological Effect in NAPRT-Negative Cancer Cell Xenograft Models (% Tumor Growth Inhibition)

Based on the effect of inhibiting proliferation limited to NAPRT-negative cancer cell lines according to the experiment of the proliferation inhibitory effect (EC₅₀) on NAPRT-negative cancer cell lines, the pharmacological effect in a xenograft model using NAPRT-negative cancer cell lines was verified. Five-week-old female BALB/c nude mice were purchased and used in the experiment after acclimation for one week. The cancer cells used in the experiment were a NAPRT-negative gastric cancer cell line HGC27, and cultured in RPMI1650 medium (Gibco) to which 5% Fetal Bovine Serum (Gibco, US) and 1% penicillin/streptomycin (Gibco) were added. HGC27 was suspended in PBS (Welgene) and transplanted to have 5×10⁶ cells into each mouse. After adding Matrigel in the same value, tumors were induced by subcutaneous injection into the right flank using a 1 mL syringe. After 3 weeks of breeding, when the average tumor size was 100 to 150 mm³, mice were randomly divided into groups of 7 and administered each compound. The compound was prepared in an excipient (0.5% DMSO/2% Tween 80/20% PEG 400/20% b-HPCD in 77.5% DW) as an agent for oral administration, and administered orally once a day from day 1 to day 21 to the mouse model in which a tumor was generated. From day 1 to day 21, the body weight was measured once daily, and the tumor size was measured three times a week. The tumor size was measured using a caliper and calculated using the following formula.

$$Tumor Volume ( {mm}^{3} ) = W \times d \times d \times 0.5 \qquad [ Mathematical Formula 2 ]$$

In Mathematical Formula 2, W is a length, and d is a width.

On day 21, all animals were euthanized, blood samples were collected, and tumors were removed. To evaluate blood toxicity, blood was collected from an EDTA-containing blood collection tube, and subjected to a general blood test (complete blood count, CBC) and a blood biochemical test. Isolated tumors were weighed after photographing and a portion was used to measure NAD to verify the on-target efficacy of the compound. To measure NAD, a NAD/NADH Quantitation Colorimetric Kit (K337) produced by BioVision was used.

The pharmacological effect of each compound in the NAPRT-negative cancer cell xenograft models, obtained in this experiment, is shown in Table 3 in the unit of % TGI (tumor growth inhibition).

TABLE 3

| Compound | Dose (mg/kg) | % TGI (Tumor Growth inhibition) |
|---|---|---|
| Example 4 | 50 mg/kg | 80% |
| Example 10 | 50 mg/kg | 61% |
| Example 11 | 50 mg/kg | 82% |
| Example 13 | 50 mg/kg | 63% |

As seen from Table 3, it was seen that the growth of cancer cells was significantly inhibited by the compounds of Examples according to the present invention.

1. A compound of Chemical Formula 1 below, an isomer thereof, a solvate thereof, a hydrate thereof, or a pharmaceutically acceptable salt thereof: 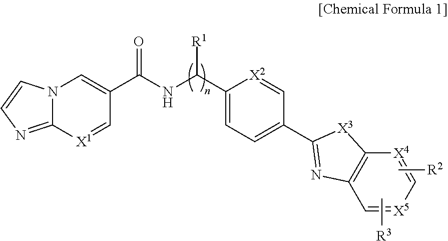
in Chemical Formula 1,
X¹ is CH or N,
X² is CH or N, and when X² is CH, it is substituted with halogen or unsubstituted,
X³ is O or S,
X⁴ and X⁵ are independently CR⁴ or N, wherein R⁴ is the same as or different from each other,
R¹ is hydrogen or C₁₋₆ alkyl, and n is 1, and
R² to R⁴ are each independently selected from the group consisting of hydrogen, halogen, amine, C₁₋₆ alkyl, C₁₋₆ haloalkyl, 5- to 10-membered aryl, 5- to 10-membered heteroaryl, —C₁₋₆ alkylene-O—R⁵, —C(O)O—R⁵, —NH—C(O)—R⁵, —NH—S(O)₂—R⁵, —S(O)₂—R⁵, —O—R⁵, and —C(O)NR⁶R⁷,
wherein R⁵ is hydrogen, C₁₋₆ alkyl, 5- to 10-membered aryl, 5- to 10-membered heteroaryl, or —C₁₋₆ alkylene-C₅₋₁₀ aryl,
R⁶ and R⁷ are each independently hydrogen, C₁₋₆ alkyl, or N, R⁶ and R⁷ are linked to each other to form 4- to 10-membered heterocycloalkyl including N.

2. The compound of claim 1, wherein the compound of Chemical Formula 1 is a compound represented by Chemical Formula 2 below, an isomer thereof, a solvate thereof, a hydrate thereof, or a pharmaceutically acceptable salt thereof: 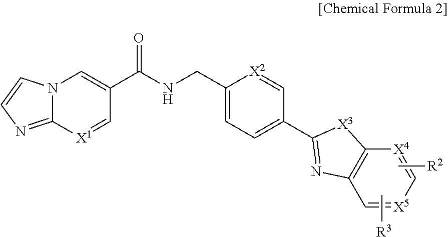
in Chemical Formula 2, X¹ to X⁵, R², and R³ are each defined as above.

3. The compound of claim 1, wherein the compound of Chemical Formula 1 is a compound represented by Chemical Formula 3 below, an isomer thereof, a solvate thereof, a hydrate thereof, or a pharmaceutically acceptable salt thereof: 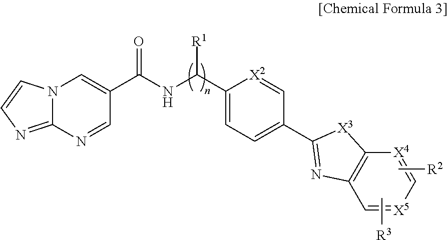
in Chemical Formula 3, X² to X⁵, n, and R¹ to R³ are each defined as above.

4. The compound of claim 1, wherein, in Chemical Formula 1, wherein X¹ is CH or N,
X² is CH or N, and when X² is CH, it is substituted with halogen or unsubstituted,
X³ is O or S,
X⁴ and X⁵ are independently CR⁴ or N, wherein R⁴ is the same as or different from each other,
R¹ is hydrogen or C₁₋₆ alkyl, and n is 1,
R² to R⁴ are each independently selected from the group consisting of hydrogen, halogen, amine, C₁₋₃ alkyl, C₁₋₃ haloalkyl, 5- or 6-membered aryl, 5- or 6-membered heteroaryl having one or more N atoms, —C₁₋₃ alkylene-O—R⁵, —C(O)O—R⁵, —NH—C(O)—R⁵, —NH—S(O)₂—R⁵, —S(O)₂—R⁵, —O—R⁵, and —C(O)NR⁶R⁷,
R⁵ is hydrogen, C₁₋₃ alkyl, 5- or 6-membered aryl, 5- or 6-membered heteroaryl having one or more N atoms, or —C₁₋₃ alkylene-C₅₋₆ aryl, and R⁶ and R⁷ are each independently hydrogen, C₁₋₃ alkyl, or N, R⁶ and R⁷ are linked to each other to form 4- to 6-membered heterocycloalkyl including N.

5. The compound of claim 1, wherein,
in —C₁₋₃alkylene-O—R⁵ of R² to R⁴, R⁵ is hydrogen,
in —C(O)O—R⁵ of R² to R⁴, R⁵ is hydrogen or C₁₋₃ alkyl,
in —NH—C(O)—R⁵ and —NH—S(O)₂—R⁵ of R² to R⁴, R⁵ is C₁₋₃ alkyl,
in —S(O)₂—R⁵ of R² to R⁴, R⁵ is C₁₋₃ alkyl or 5- or 6-membered aryl, and
in —O—R⁵ of R² to R⁴, R⁵ is hydrogen, C₁₋₃ alkyl, —C₁₋₃ alkylene-C₅₋₆ aryl, 5- or 6-membered aryl, or 5- or 6-membered heteroaryl.

6. The compound of claim 1, wherein, when X³ is S, R² to R⁴ are each independently hydrogen, C₁₋₆ alkyl, C₁₋₆ alkoxy, or halogen.

7. The compound of claim 1, wherein the compound of Chemical Formula 1 is any one selected from the group consisting of the following compounds:
1) N-[[4-(6-methyl-1,3-benzoxazol-2-yl)phenyl]methyl]imidazo[1,2-a]pyrimidin-6-carboxamide; 2) N-[[4-(1,3-benzoxazol-2-yl)phenyl]methyl]imidazo[1,2-a]pyrimidin-6-carboxamide; 3) N-[[4-(1,3-benzoxazol-2-yl)phenyl]methyl]imidazo[1,2-a]pyrimidin-6-carboxamide; 4) N-methyl]imidazo[1,2-a]pyrimidin-6-carboxamide; 5) 2-[4-[(imidazo[1,2-a]pyrimidin-6-carbonylamino)methyl]phenyl]-1,3-benzoxazol-6-carboxylic acid; 6) N-[[4-(5-methyl-1,3-benzoxazol-2-yl)phenyl]methyl]imidazo[1,2-a]pyrimidin-6-carboxamide; 7) N-[[4-(5-methoxy-1,3-benzoxazol-2-yl)phenyl]methyl]imidazo[1,2-a]pyrimidin-6-carboxamide; 8) N-[[4-(6-methyl-1,3-benzothiazol-2-yl)phenyl]methyl]imidazo[1,2-a]pyrimidin-6-carboxamide; 9) N-[[4-(5-methylsulfonyl-1,3-benzoxazol-2-yl)phenyl]methyl]imidazo[1,2-a]pyrimidin-6-carboxamide; 10) N-[[4-(6-chloro-5-methyl-1,3-benzoxazol-2-yl)phenyl]methyl]imidazo[1,2-a]pyrimidin-6-carboxamide; 11) N-[[4-(6-fluoro-5-methyl-1,3-benzoxazol-2-yl)phenyl]methyl]imidazo[1,2-a]pyrimidin-6-carboxamide; 12) N-[[4-(5-methylsulfonyl-1,3-benzoxazol-2-yl)phenyl]methyl]imidazo[1,2-a]pyridin-6-carboxamide; 13) N-[[4-[5-(trifluoromethyl)-1,3-benzoxazol-2-yl]phenyl]methyl]imidazo[1,2-a]pyrimidin-6-carboxamide; 14) N-[[4-(6-ethylsulfonyl-1,3-benzoxazol-2-yl)phenyl]methyl]imidazo[1,2-a]pyrimidin-6-carboxamide; 15) N-[[4-[5-(propanoylamino)-1,3-benzoxazol-2-yl]phenyl]methyl]imidazo[1,2-a]pyrimidin-6-carboxamide; 16) N-[[4-(5-chloro-1,3-benzoxazol-2-yl)phenyl]methyl]imidazo[1,2-a]pyrimidin-6-carboxamide; 17) N-[[4-(6-fluoro-1,3-benzoxazol-2-yl)phenyl]methyl]imidazo[1,2-a]pyrimidin-6-carboxamide; 18) N-[[4-(5-fluoro-1,3-benzoxazol-2-yl)phenyl]methyl]imidazo[1,2-a]pyrimidin-6-carboxamide; 19) N-[[4-[6-(trifluoromethyl)-1,3-benzoxazol-2-yl]phenyl]methyl]imidazo[1,2-a]pyrimidin-6-carboxamide; 20) N-[[4-(4-methyl-1,3-benzoxazol-2-yl)phenyl]methyl]imidazo[1,2-a]pyrimidin-6-carboxamide; 21) N-[[4-(7-methyl-1,3-benzoxazol-2-yl)phenyl]methyl]imidazo[1,2-a]pyrimidin-6-carboxamide; 22) N-[[4-(6-methoxy-1,3-benzoxazol-2-yl)phenyl]methyl]imidazo[1,2-a]pyrimidin-6-carboxamide; 23) N-[[4-(5-hydroxy-1,3-benzoxazol-2-yl)phenyl]methyl]imidazo[1,2-a]pyrimidin-6-carboxamide; 24) N-[[4-(5-methyloxazolo[5,4-b]pyridin-2-yl)phenyl]methyl]imidazo[1,2-a]pyrimidin-6-carboxamide; 25) N-[[4-[5-(2-pyridyloxy)-1,3-benzoxazol-2-yl]phenyl]methyl]imidazo[1,2-a]pyrimidin-6-carboxamide; 26) N-[[4-(6-amino-1,3-benzoxazol-2-yl)phenyl]methyl]imidazo[1,2-a]pyrimidin-6-carboxamide; 27) N-[[4-[5-(3-pyridyl)-1,3-benzoxazol-2-yl]phenyl]methyl]imidazo[1,2-a]pyrimidin-6-carboxamide; 28) N-[[5-(6-methyl-1,3-benzoxazol-2-yl)-2-pyridyl]methyl]imidazo[1,2-a]pyrimidin-6-carboxamide; 29) ethyl 2-[4-[(imidazo[1,2-a]pyrimidin-6-carbonylamino)methyl]phenyl]-1,3-benzoxazol-5-carboxylate; 30) N-[[4-[5-(hydroxymethyl)-1,3-benzoxazol-2-yl]phenyl]methyl]imidazo[1,2-a]pyrimidin-6-carboxamide; 31) N-ethyl-2-[4-[(imidazo[1,2-a]pyrimidin-6-carbonylamino)methyl]phenyl]-1,3-benzoxazol-5-carboxamide; 32) 2-[4-[(imidazo[1,2-a]pyrimidin-6-carbonylamino)methyl]phenyl]-N,N-dimethyl-1,3-benzoxazol-5-carboxamide; 33) N-[[4-[5-(azetidin-1-carbonyl)-1,3-benzoxazol-2-yl]phenyl]methyl]imidazo[1,2-a]pyrimidin-6-carboxamide; 34) N-[[4-[5-(pyrrolidine-1-carbonyl)-1,3-benzoxazol-2-yl]phenyl]methyl]imidazo[1,2-a]pyrimidin-6-carboxamide; 35) N-[[2-fluoro-4-(6-methyl-1,3-benzoxazol-2-yl)phenyl]methyl]imidazo[1,2-a]pyrimidin-6-carboxamide; 36) N-[(4-oxazolo[4,5-c]pyridin-2-ylphenyl)methyl]imidazo[1,2-a]pyrimidin-6-carboxamide; 37) N-[[4-(5-ethylsulfonyl-1,3-benzoxazol-2-yl)phenyl]methyl]imidazo[1,2-a]pyrimidin-6-carboxamide; 38) N-[[4-[5-(benzenesulfonyl)-1,3-benzoxazol-2-yl]phenyl]methyl]imidazo[1,2-a]pyrimidin-6-carboxamide; 39) N-[[4-(5,6-difluoro-1,3-benzoxazol-2-yl)phenyl]methyl]imidazo[1,2-a]pyrimidin-6-carboxamide; 40) N-[[4-(4,6-difluoro-1,3-benzoxazol-2-yl)phenyl]methyl]imidazo[1,2-a]pyrimidin-6-carboxamide; 41) N-[[4-[6-chloro-5-(trifluoromethyl)-1,3-benzoxazol-2-yl]phenyl]methyl]imidazo[1,2-a]pyrimidin-6-carboxamide; 42) N-[[4-[5-chloro-6-(trifluoromethyl)-1,3-benzoxazol-2-yl]phenyl]methyl]imidazo[1,2-a]pyrimidin-6-carboxamide; 43) N-[[4-(5-methoxy-6-methyl-1,3-benzoxazol-2-yl)phenyl]methyl]imidazo[1,2-a]pyrimidin-6-carboxamide; 44) N-[[4-(5-ethyl-1,3-benzoxazol-2-yl)phenyl]methyl]imidazo[1,2-a]pyrimidin-6-carboxamide; 45) N-[[4-(6-ethyl-1,3-benzoxazol-2-yl)phenyl]methyl]imidazo[1,2-a]pyrimidin-6-carboxamide; 46) N-[[4-(5-amino-1,3-benzoxazol-2-yl)phenyl]methyl]imidazo[1,2-a]pyrimidin-6-carboxamide; 47) N-[[4-[5-(methanesulfonamino)-1,3-benzoxazol-2-yl]phenyl]methyl]imidazo [1,2-a]pyrimidin-6-carboxamide; 48) N-[[4-(5-methyl-1,3-benzothiazol-2-yl)phenyl]methyl]imidazo[1,2-a]pyrimidin-6-carboxamide; 49) N-[[4-(5-chloro-1,3-benzothiazol-2-yl)phenyl]methyl]imidazo[1,2-a]pyrimidin-6-carboxamide; 50) N-[[4-(6-chloro-1,3-benzothiazol-2-yl)phenyl]methyl]imidazo[1,2-a]pyrimidin-6-carboxamide; 51) N-[[4-(5-methoxy-1,3-benzothiazol-2-yl)phenyl]methyl]imidazo[1,2-a]pyrimidin-6-carboxamide; 52) N-[[4-(6-methoxy-1,3-benzothiazol-2-yl)phenyl]methyl]imidazo[1,2-a]pyrimidin-6-carboxamide; 53) N-[[4-(5-phenoxy-1,3-benzoxazol-2-yl)phenyl]methyl]imidazo[1,2-a]pyrimidin-6-carboxamide; 54) N-[[4-(5-propoxy-1,3-benzoxazol-2-yl)phenyl]methyl]imidazo[1,2-a]pyrimidin-6-carboxamide; 55) N-[[4-(5-benzyloxy-1,3-benzoxazol-2-yl)phenyl]methyl]imidazo[1,2-a]pyrimidin-6-carboxamide; 56) N-[[4-(5-phenyl-1,3-benzoxazol-2-yl)phenyl]methyl]imidazo[1,2-a]pyrimidin-6-carboxamide; 57) N-[[4-(6-phenoxy-1,3-benzoxazol-2-yl)phenyl]methyl]imidazo[1,2-a]pyrimidin-6-carboxamide; 58) N-[[4-[6-(3-pyridyloxy)-1,3-benzoxazol-2-yl]phenyl]methyl]imidazo[1,2-a]pyrimidin-6-carboxamide; 59) N-[[4-(6-phenyl-1,3-benzoxazol-2-yl)phenyl]methyl]imidazo[1,2-a]pyrimidin-6-carboxamide; 60) N-[[4-(5-chlorooxazolo[5,4-b]pyridin-2-yl)phenyl]methyl]imidazo[1,2-a]pyrimidin-6-carboxamide; 61) N-[[4-(6-methyl-1,3-benzoxazol-2-yl)phenyl]methyl]imidazo[1,2-a]pyridin-6-carboxamide; 62) N-[[4-(6-chlorooxazolo[4,5-c]pyridin-2-yl)phenyl]methyl]imidazo[1,2-a]pyridin-6-carboxamide; 63) N-[[5-(6-chloro-1,3-benzoxazol-2-yl)-2-pyridyl]methyl]imidazo[1,2-a]pyrimidin-6-carboxamide; and 64) N-[1-[4-(6-methyl-1,3-benzooxazol-2-yl)phenyl]ethyl]imidazo[1,2-a]pyrimidin-6-carboxamide.

8. A method of preparing a compound of Chemical Formula 1 in which X³ is O, comprising, as shown in Reaction Scheme 1 below,
reacting a compound of Chemical Formula 4 with a compound of Chemical Formula 5 to prepare a compound of Chemical Formula 6; and reacting the compound of Chemical Formula 6 with a compound of Chemical Formula 7 to prepare a compound of Chemical Formula 1: 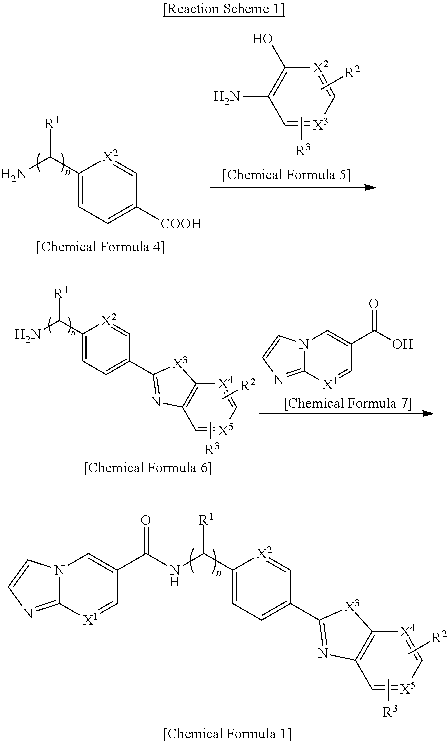
in this scheme, X¹ to X⁵, n, and R¹ to R³ are each defined as above.

9. A method of preparing a compound of Chemical Formula 1 in which X³ is S, comprising, as shown in Reaction Scheme 2 below,
reacting a compound of Chemical Formula 8 with a compound of Chemical Formula 9 to prepare the compound of Chemical Formula 6; and reacting the compound of Chemical Formula 6 and the compound of Chemical Formula 7 to prepare the compound of Chemical Formula 1: 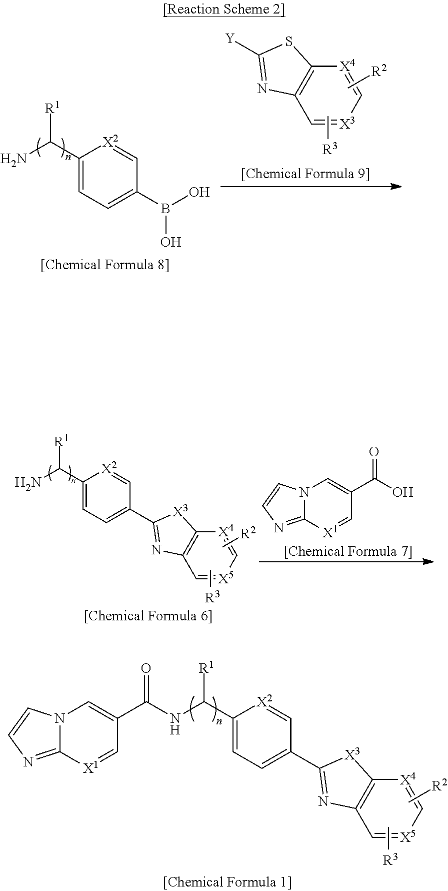
in this scheme, each of X¹ to X⁵, and R¹ to R³ is defined as above, and Y is a leaving group.

10. A pharmaceutical composition for preventing or treating a nicotinamide phosphoribosyl transferase (NamPT)-related disease, comprising:
the compound of Chemical Formula 1 of claim 1, an isomer thereof a solvate thereof, a hydrate thereof, or a pharmaceutically acceptable salt thereof as an active ingredient; and a pharmaceutically acceptable carrier.

11. The pharmaceutical composition of claim 10, wherein the NamPT-related disease is one or more selected from the group consisting of cancer, viral infection, human immunodeficient virus, hepatitis virus, herpes virus, herpes simplex, inflammatory disorder, irritable bowel syndrome, inflammatory bowel disease, rheumatoid arthritis, asthma, chronic obstructive pulmonary disease, osteoarthritis, osteoporosis, dermatitis, atopic dermatitis, psoriasis, systemic lupus erythematosus, multiple sclerosis, psoriatic arthritis, ankylosing spondylitis, graft-versus-host disease, Alzheimer's disease, cerebrovascular injury, atherosclerosis, diabetes, glomerulonephritis, and metabolic syndrome.

12. The pharmaceutical composition of claim 11, wherein the cancer is one or more selected from the group consisting of liver cancer, bile duct cancer, gall bladder cancer, esophageal cancer, stomach cancer, ovarian cancer, breast cancer, uterine cancer, colon cancer, rectal cancer, cervical cancer, prostate cancer, skin cancer, pancreatic cancer, leukemia, lymphoma, Hodgkin's disease, lung cancer, bronchial cancer, multiple myeloma, squamous cell carcinoma, kidney cancer, urethral cancer, bladder cancer, head and neck cancer, brain cancer, and central nervous system cancer.

13. A method of preventing or treating a NamPT-related disease comprising administering a therapeutically effective amount of a compound of Chemical Formula 1 of claim 1, an isomer thereof, a solvate thereof, a hydrate thereof, or a pharmaceutically acceptable salt thereof to a subject in need thereof.

14. A method of claim 13, wherein the NamPT-related disease is one or more selected from the group consisting of cancer, viral infection, human immunodeficient virus, hepatitis virus, herpes virus, herpes simplex, inflammatory disorder, irritable bowel syndrome, inflammatory bowel disease, rheumatoid arthritis, asthma, chronic obstructive pulmonary disease, osteoarthritis, osteoporosis, dermatitis, atopic dermatitis, psoriasis, systemic lupus erythematosus, multiple sclerosis, psoriatic arthritis, ankylosing spondylitis, graft-versus-host disease, Alzheimer's disease, cerebrovascular injury, atherosclerosis, diabetes, glomerulonephritis, and metabolic syndrome.

15. A method of claim 13, wherein the cancer is one or more selected from the group consisting of liver cancer, bile duct cancer, gall bladder cancer, esophageal cancer, stomach cancer, ovarian cancer, breast cancer, uterine cancer, colon cancer, rectal cancer, cervical cancer, prostate cancer, skin cancer, pancreatic cancer, leukemia, lymphoma, Hodgkin's disease, lung cancer, bronchial cancer, multiple myeloma, squamous cell carcinoma, kidney cancer, urethral cancer, bladder cancer, head and neck cancer, brain cancer, and central nervous system cancer.
